(19) United States
(12) Patent Application Publication
(10) Pub. No.: US 2019/0231771 A1
(43) Pub. Date: Aug. 1, 2019

(54) METHODS FOR TREATMENT AND PREVENTION OF OPIOID INDUCED CONSTIPATION USING ORAL COMPOSITIONS OF METHYLNALTREXONE

(71) Applicant: Salix Pharmaceuticals, Ltd., Raleigh, NC (US)

(72) Inventor: Enoch Bortey, Chapel Hill, NC (US)

(73) Assignee: Salix Pharmaceuticals, Ltd., Raleigh, NC (US)

(21) Appl. No.: 16/206,570

(22) Filed: Nov. 30, 2018

Related U.S. Application Data
(63) Continuation of application No. 13/720,235, filed on Dec. 19, 2012, now abandoned.
(60) Provisional application No. 61/577,654, filed on Dec. 19, 2011.

(51) Int. Cl. A61K 31/485; A61K 45/06

(52) U.S. Cl. CPC A61K 31/485; A61K 45/06

(57) ABSTRACT

Presented herein are methods for treatment or prevention of opioid induced constipation by administration of oral compositions of methylnaltrexone. The methods are based, at least in part, on the identification of subjects that are particularly susceptible to such treatment and optimal dosages of such oral compositions to treat or prevent opioid induced constipation and, further, to minimize the occurrence of adverse events associated with such treatment.

### RELATED APPLICATIONS

This application is a continuation of U.S. patent application Ser. No. 13/720,235, filed Dec. 19, 2012, which claims the benefit of U.S. Provisional Application No. 61/577,654, filed Dec. 19, 2011, the entire contents of which are hereby incorporated herein by reference.

### BACKGROUND

Opioids are widely used in treating patients with pain. Such patients include those with advanced cancers and other terminal diseases and also those with chronic non-malignant pain and acute non-malignant pain. Opioids are narcotic medications that activate opioid receptors located in the central nervous system to relieve pain. Opioids, however, also react with receptors outside of the central nervous system, resulting in side effects including constipation, nausea, vomiting, urinary retention, and severe itching. Notable are the effects of opioids in the gastrointestinal (GI) tract where these drugs inhibit gastric emptying and peristalsis in the intestines, thereby decreasing the rate of intestinal transit and producing constipation. The use of opioids in treating pain is often limited due to these undesired side effects, which can be debilitating and often cause patients to refuse the use of opioid analgesics.

In addition to exogenous opioid-induced side effects, studies have suggested that endogenous opioids and opioid receptors may also affect the gastrointestinal (GI) tract and may be involved in normal regulation of intestinal motility and mucosal transport of fluids. Thus, an abnormal physiological level of endogenous opioids and/or receptor activity may also lead to bowel dysfunction. For example, patients who have undergone surgical procedures, especially surgery of the abdomen, often suffer from a particular bowel dysfunction, termed post-operative ileus, that may be caused by fluctuations in natural opioid levels. Similarly, women who have recently given birth commonly suffer from post partum ileus, which may be caused by similar fluctuations in natural opioid levels as a result of birthing stress. Gastrointestinal dysfunction associated with post-operative or post-partum ileus can typically last for 3 to 5 days, with some severe cases lasting more than a week. Administration of opioids to a patient after surgery to treat pain, which is now an almost universal practice, may exacerbate bowel dysfunction, thereby delaying recovery of normal bowel function, prolonging hospital stays, and increasing medical care costs.

Opioid receptor antagonists, such as naloxone, naltrexone, and nalmefene, have been studied as a means of antagonizing the undesirable peripheral side effects of opioids. However, these agents not only act on peripheral opioid receptors but also on opioid receptors in the central nervous system, sometimes reversing the beneficial and desired analgesic effects of opioids or causing symptoms of opioid withdrawal. Preferable approaches for use in controlling opioid-induced side effects include administration of peripheral acting opioid receptor antagonists that do not readily cross the blood-brain barrier.

The peripheral μ opioid receptor antagonist methylnaltrexone has been studied since the late 1970s. It has been used in patients to reduce opioid-induced side effects such as constipation, pruritus, nausea, and urinary retention (see, e.g., U.S. Pat. Nos. 5,972,954, 5,102,887, 4,861,781, and 4,719,215; and Yuan et al., Drug and Alcohol Dependence 1998, 52, 161). The dosage form of methylnaltrexone used most often in these studies has been a solution of methylnaltrexone for intravenous injection.

### SUMMARY

Presented herein are methods for treatment or prevention of opioid induced constipation by administration of oral compositions of methylnaltrexone. The present invention is based, at least in part, on the identification of subjects that are particularly susceptible to such treatment and optimal dosages of such oral compositions to treat or prevent opioid induced constipation and, further, to minimize the occurrence of adverse events associated with such treatment.

Accordingly, presented herein are methods of treating a subject having opioid induced constipation, comprising orally administering to the subject a pharmaceutical composition comprising a salt of formula (I):

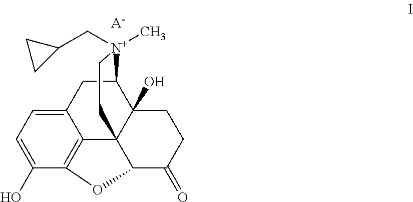

wherein A⁻ is an anion of an amphiphilic pharmaceutically acceptable excipient, wherein the administration of the pharmaceutical composition results in a rescue free bowel movement; thereby treating the subject.

In another aspect, provided herein are methods of preventing a subject from having opioid induced constipation, comprising orally administering to the subject a pharmaceutical composition comprising a salt of formula (I):

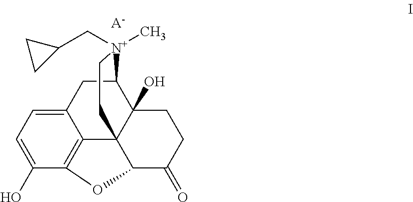

wherein A⁻ is an anion of an amphiphilic pharmaceutically acceptable excipient, thereby preventing the subject from having opioid induced constipation.

In one embodiment, A⁻ is sodium dodecyl (lauryl) sulfate.

In another embodiment, the pharmaceutical composition comprises a combination of a first salt comprising methylnaltrexone and bromide, and a second salt comprising methylnaltrexone and sodium dodecyl (lauryl) sulfate.

In another embodiment, the pharmaceutical composition comprises about 150 mg of methylnaltrexone, or a salt thereof.

In another embodiment, the pharmaceutical composition further comprises at least one agent selected from the group consisting of sodium bicarbonate, microcrystalline cellulose, crospovidone, polysorbate 80, edetate calcium disodium dehydrate, silicified microcrystalline cellulose, talc, colloidal silicon dioxide, magnesium stearate, and combinations thereof.

In another embodiment, the pharmaceutical composition is a tablet.

In one embodiment, the methods comprise orally administering about 150 mg of methylnaltrexone, or a salt thereof. In a related embodiment, the about 150 mg of methylnaltrexone is administered as one tablet comprising about 150 mg of methylnaltrexone.

In one embodiment, the methods comprise orally administering about 300 mg of methylnaltrexone, or a salt thereof. In a related embodiment, the about 300 mg of methylnaltrexone is administered as two tablets each comprising about 150 mg of methylnaltrexone.

In one embodiment, the methods comprise orally administering about 450 mg of methylnaltrexone, or a salt thereof. In one embodiment, the about 450 mg of methylnaltrexone is administered as three tablets each comprising about 150 mg of methylnaltrexone.

In one embodiment, the subject has chronic non-malignant pain.

In another embodiment, the subject has had chronic non-malignant pain for at least 2 months prior to administration of the pharmaceutical composition.

In one embodiment, the subject has been receiving opioid treatment prior to administration of the pharmaceutical composition. In a related embodiment, the subject has been receiving opioid treatment for at least one month.

In another embodiment, the subject has been receiving opioid treatment comprising at least 50 mg of oral morphine equivalents per day for at least 14 days.

In one embodiment, the subject will start opioid treatment in less than 1, 2, 3 or 4 weeks.

In one embodiment, the subject has had opioid induced constipation for at least 30 days.

In another embodiment, the subject has experienced less than 3 rescue free bowel movements per week for at least four consecutive weeks.

In one embodiment, the subject has experienced straining during bowel movements.

In another embodiment, the subject has experienced incomplete evacuation.

In one embodiment, the subject has experienced a Bristol Stool Form Scale type 1 or 2 for at least 25% of rescue free bowel movements.

In one embodiment, the methods result in a rescue free bowel movement within 4 hours of administration of the pharmaceutical composition.

In another embodiment, the methods result in an increase of at least one rescue free bowel movement per week as compared to the number of rescue free bowel movements per week prior to administration of the pharmaceutical composition.

In another embodiment, the methods result in an increase of at least 2, 3, 4 or 5 rescue free bowel movements per week.

In another embodiment, the methods result in an increase of at least one rescue free bowel movement per week for each of the first 4 weeks of daily administration of the pharmaceutical composition.

In another embodiment, the subject experiences at least 3 rescue free bowel movements in each of the first 4 weeks of daily administration of the pharmaceutical composition; and the subject experiences an increase of at least one rescue free bowel movement per week for at least 3 of the first 4 weeks of daily administration as compared to the number of rescue free bowel movements per week prior to administration of the pharmaceutical composition.

In another aspect, provided herein are methods of eliciting a rescue free bowel movement in a subject suffering from opioid induced constipation, comprising orally administering to the subject a pharmaceutical composition comprising a salt of formula (I):

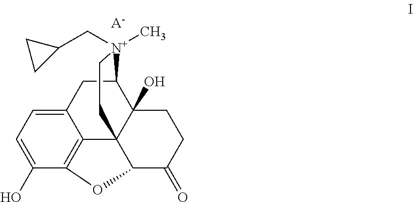

wherein A⁻ is an anion of an amphiphilic pharmaceutically acceptable excipient, thereby eliciting a rescue free bowel movement. In one embodiment, the method elicits a rescue free bowel movement within 4 hours of administration.

In another aspect, provided herein are methods of increasing the number of rescue free bowel movements experienced by a subject, comprising orally administering to the subject a pharmaceutical composition comprising a salt of formula (I):

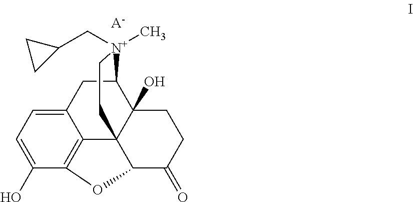

wherein A⁻ is an anion of an amphiphilic pharmaceutically acceptable excipient, thereby increasing the number of rescue free bowel movements experienced by the subject.

In one embodiment, the subject is administered the pharmaceutical composition at least once a day for at least four weeks.

In another embodiment, the subject experiences an increase of at least one rescue free bowel movement for at least 3 out of the four weeks and wherein the subject experiences at least 3 rescue free bowel movements for each of the four weeks.

In one embodiment, the number of rescue free bowel movements increases each of the four weeks as compared to the number of rescue free bowel movements experienced by the subject prior to administration.

In another aspect, provided herein are of assessing the efficacy of the pharmaceutical composition disclosed herein for treating a subject suffering from opioid induced constipation, comprising orally administering to the subject a pharmaceutical composition comprising a salt of formula (I):

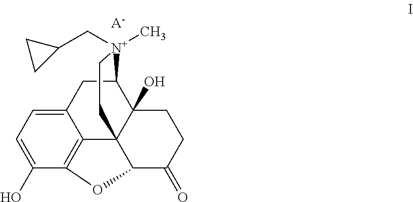

wherein A⁻ is an anion of an amphiphilic pharmaceutically acceptable excipient, wherein at least one of:

(i) a rescue free bowel movement within four hours of administration of the pharmaceutical composition;

(ii) an increase in the number of rescue free bowel movements per week upon daily administration of the pharmaceutical composition as compared to the number of rescue free bowel movements per week prior to daily administration of the pharmaceutical composition;

or

(iii) an increase in the number of rescue free bowel movements per week upon daily administration of the pharmaceutical composition as compared to the number of rescue free bowel movements per week prior to administration of the pharmaceutical composition in at least three of the first four weeks of daily administration; and at least three rescue free bowel movements per week for the first four weeks of daily administration;

is indicative of the efficacy of the pharmaceutical composition.

In another aspect, provided herein are methods for treating a subject having opioid induced constipation, comprising identifying if the subject:

(i) has chronic non-malignant pain;

(ii) has had chronic non-malignant pain for at least 2 months;

(iii) has been receiving opioid treatment;

(iv) has been receiving opioid treatment for at least one month;

(v) has been receiving opioid treatment comprising at least 50 mg of oral morphine equivalents per day for at least 14 days;

(vi) has opioid induced constipation;

(vii) has had opioid induced constipation for at least 30 days;

(viii) has had less than 3 rescue free bowel movements per week for at least four consecutive weeks;

(ix) has experienced straining during bowel movements;

(x) has experienced incomplete evacuation;

(xi) has experienced a Bristol Stool Form Scale type 1 or 2 for at least 25% of rescue free bowel movements;

(xii) has no history of chronic constipation prior to initiation of opioid therapy; or

(xiii) any combination of (i)-(xii); and orally administering to the subject a pharmaceutical composition comprising a salt of formula (I):

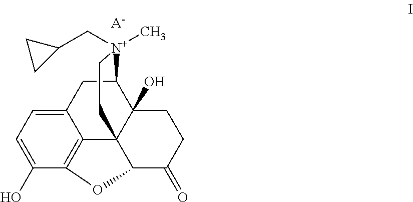

wherein A⁻ is an anion of an amphiphilic pharmaceutically acceptable excipient, wherein the subject exhibits any one of (i)-(x).

In another aspect, provided herein are methods of reducing the occurrence of adverse events associated with treatment of opioid induced constipation, comprising orally administering to the subject a pharmaceutical composition comprising a salt of formula (I):

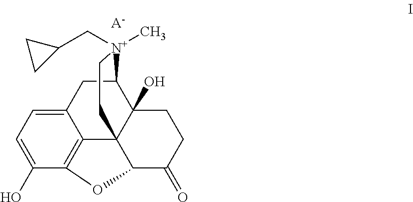

wherein A⁻ is an anion of an amphiphilic pharmaceutically acceptable excipient, wherein the pharmaceutical composition reduces the occurrence of adverse events as compared to a pharmaceutical composition not comprising an anion of amphiphilic pharmaceutically acceptable excipient.

In one embodiment, A⁻ is sodium dodecyl (lauryl) sulfate.

In another embodiment, the pharmaceutical composition comprises a combination of a first salt comprising methylnaltrexone and bromide, and a second salt comprising methylnaltrexone and sodium dodecyl (lauryl) sulfate.

In one embodiment, the pharmaceutical composition comprises about 150 mg of methylnaltrexone, or a salt thereof.

In another embodiment, the pharmaceutical composition further comprises at least one agent selected from the group consisting of sodium bicarbonate, microcrystalline cellulose, crospovidone, polysorbate 80, edetate calcium disodium dehydrate, silicified microcrystalline cellulose, talc, colloidal silicon dioxide, magnesium stearate, and combinations thereof.

In another embodiment, the pharmaceutical composition is a tablet.

In one embodiment, the methods comprise orally administering about 150 mg of methylnaltrexone, or a salt thereof. In a related embodiment, the about 150 mg of methylnaltrexone is administered as one tablet comprising about 150 mg of methylnaltrexone.

In one embodiment, the methods comprise orally administering about 300 mg of methylnaltrexone, or a salt thereof. In a related embodiment, the about 300 mg of methylnaltrexone is administered as two tablets each comprising about 150 mg of methylnaltrexone.

In one embodiment, the methods comprise orally administering about 450 mg of methylnaltrexone, or a salt thereof. In one embodiment, the about 450 mg of methylnaltrexone is administered as three tablets each comprising about 150 mg of methylnaltrexone.

In another aspect, provided herein are methods treating a subject having opioid induced constipation, comprising the steps of

(a) orally administering to the subject a pharmaceutical composition comprising about 150 mg of methylnaltrexone, or a salt thereof, and sodium dodecyl (lauryl) sulfate;

(b) determining whether the composition treats the subject, wherein at least one response selected from the group consisting of (i)-(iii) indicates that the composition treats the subject:
(i) a rescue free bowel movement within four hours of administration of the pharmaceutical composition;
(ii) an increase in the number of rescue free bowel movements per week upon daily administration of the pharmaceutical composition as compared to the number of rescue free bowel movements per week prior to daily administration of the pharmaceutical composition; or
(iii) an increase in the number of rescue free bowel movements per week upon daily administration of the pharmaceutical composition as compared to the number of rescue free bowel movements per week prior to administration of the pharmaceutical composition in at least three of the first four weeks of daily administration; and at least three rescue free bowel movements per week for the first four weeks of daily administration;

(c) orally administering a pharmaceutical composition comprising 300 mg or 450 mg of methylnaltrexone, or a salt thereof, and sodium dodecyl (lauryl) sulfate, if the subject does not exhibit a response selected from the group consisting of (b)(i)-(iii) following step (a).

In another aspect, provided herein are methods of treating a subject having opioid induced constipation, comprising orally administering a pharmaceutical composition comprising methylnaltrexone, or a salt thereof, wherein the pharmaceutical composition comprises a salt of formula (I):

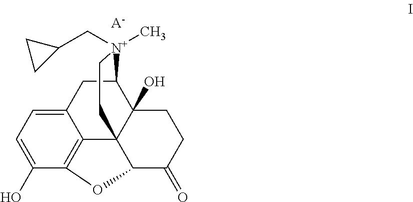

wherein A⁻ is an anion of an amphiphilic pharmaceutically acceptable excipient, wherein the composition provides a dose in the range of about 300 mg to about 400 mg of methylnaltrexone or salt thereof; wherein (i) the method results in a rescue free bowel movement within 4 hours of administration of the pharmaceutical composition; and (ii) the result is sustainable for at least 4 weeks with daily administration.

In one embodiment, the methods further provide the subject (i) at least 3 rescue free bowel movements per week for at least 3 of 4 weeks of daily administration of the pharmaceutical composition; and (ii) the subject experiences an increase of at least one rescue free bowel movement per week as compared to the number of rescue free bowel movements per week prior to administration of the pharmaceutical composition.

In another aspect, provided herein are methods of increasing the bioavailability of MNTX and its metabolites in a subject comprising administering MNTX to a subject orally.

In one embodiment, the MNTX is administered orally from between 1 and 7 days. In one embodiment, the MNTX is administered orally from between 1 and 28 days.

In one embodiment, AUC and Cₘₐₓ of one or more of MNTX and its metabolites are increased in a subject as compared to the AUC and Cₘₐₓ of a subject administered a lesser amount of MNTX via subcutaneous injections.

In one embodiment, MNTX administered orally has a higher accumulation values for one or more of MNTX, M2, M4 or M5 as compared to a subject administered a lesser amount of MNTX via subcutaneous injections.

In one embodiment, the accumulation values following oral administration comprise about 1.20 for MNTX. In one embodiment, accumulation values following oral administration comprise about 1.30 for M2. In one embodiment, the accumulation values following oral administration comprise about 1.62 for M4. In one embodiment, the accumulation values following oral administration comprise about 1.76 for M5. In one embodiment, the accumulation values following oral administration comprise about 1.20 for MNTX, about 1.30 for M2, about 1.62 for M4 and about 1.76 for M5.

In another aspect, provided herein are methods of increasing the bioavailability of MNTX, comprising administering MNTX without food to a subject in need thereof.

In one embodiment, the MNTX is administered orally 450 mg once a day. In one embodiment, the MNTX is administered as 3×150 mg tablets.

In one embodiment, the MNTX is administered at least about 10 hours after the subject's last meal. In one embodiment, the the subject is identified as not having had a meal within 10 hours. In one embodiment, the MNTX is administered at least about four hours prior to the subject's next meal. In one embodiment, the the subject is instructed to avoid a high-fat and/or high-caloric meal for at least about 10 hours prior to and for about four hours after administration of MNTX.

In one embodiment, the administration with food significantly delays MNTX absorption.

In one embodiment, taking MNTX without food increases systemic absorption from between half and three quarters compared to taking MNTX with food. In one embodiment, taking MNTX without food decreases Tₘₐₓ from between about 35% and 60%_as compared to taking MNTX with food. In one embodiment, the taking MNTX without food increases Cₘₐₓ from between 1- and 3-fold as compared to taking MNTX with food. In one embodiment, the taking MNTX without food increases AUC from between 1- and 3-fold as compared to taking MNTX with food.

In another aspect, provided herein are methods of increasing the laxation effect of MNTX, comprising administering MNTX without food to a subject in need thereof.

In one embodiment, 450 mg MNTX is administered orally once a day. In one embodiment, MNTX is administered as 3×150 mg tablets. In one embodiment, MNTX is administered at least about 10 hours after the subject's last meal. In one embodiment, MNTX is administered at least about four hours prior to the subject's next meal.

In one embodiment, the subject is instructed to avoid a high-fat and/or high-caloric meal for at least about 10 hours prior to and for about four hours after administration of MNTX. In one embodiment, n the subject is identified as not having had a meal within 10 hours.

### BRIEF DESCRIPTION OF THE DRAWINGS

FIG. 1 depicts the average proportion of rescue free bowel movements per subject within four hours of all doses within the first four weeks of administration of study drug (MNTX3201), in accordance with Example 1, as compared to MNTX3356 formulation.

FIG. 2 depicts a Kaplan Meier Curve for time to rescue free bowel movement following first dose of study drug (MNTX3201), in accordance with Example 1, as compared to the MNTX3356 formulation.

FIG. 3 depicts the average proportion of rescue free bowel movements per subject within four hours of all doses within the first four weeks of administration of study drug (MNTX3201), in accordance with Example 1, as compared to 3200A3-2201-US Oral IR Tab, 3200A3-2202-WW Oral IR Cap, and 3200A3-200-WW Oral Capsule.

FIGS. 4A, 4B and 4C depict Kaplan Meier curves for time to rescue free bowel movement following first dose of study drug (MNTX3201), in accordance with Example 1, as compared to each of 3200A3-2201-US Oral IR Tab (FIG. 4A), 3200A3-2202-WW Oral IR Cap (FIG. 4B), and 3200A3-200-WW Oral Capsule (FIG. 4A), respectively.

FIG. 5 (Table 1) provides a summary of subject disposition, e.g., ineligibility, protocol violation, etc., for subjects enrolled in the study as set forth in Example 1.

FIG. 6 (Table 2) provides the demographics for all subjects enrolled in the study as set forth in Example 1.

FIG. 7 (Table 3) provides the baseline disease characteristics for all subjects enrolled in the study. Specifically, FIG. 7 provides the nature of the non-malignant chronic pain experienced by the subject, the average number of rescue free bowel movements per week for each subject and the average number of subjects having less than 3 rescue free bowel movements per week.

FIG. 8 (Table 4) provides data related to the primary efficacy endpoint, i.e., the average proportion of rescue free bowel movements per subject within 4 hours of all doses during the first 4 weeks of the study as set forth in Example 1.

FIG. 9 (Table 5) provides data related to the primary efficacy endpoint specific for male subjects, i.e., the average proportion of rescue free bowel movements per male subject within 4 hours of all doses during the first 4 weeks of the study as set forth in Example 1.

FIG. 10 (Table 6) provides data related to the primary efficacy endpoint specific for female subjects, i.e., the average proportion of rescue free bowel movements per female subject within 4 hours of all doses during the first 4 weeks of the study as set forth in Example 1.

FIG. 11 (Table 7) provides data related to the primary efficacy endpoint specific for subjects 65 years of age or younger, i.e., the average proportion of rescue free bowel movements per subject 65 years or younger within 4 hours of all doses during the first 4 weeks of the study as set forth in Example 1.

FIG. 12 (Table 8) provides data related to the primary efficacy endpoint specific for subjects older than 65 years of age, i.e., the average proportion of rescue free bowel movements per subject older than 65 years of age within 4 hours of all doses during the first 4 weeks of the study as set forth in Example 1.

FIG. 13 (Table 9) provides data related to the primary efficacy endpoint specific for subjects weighing less than 86 kg, i.e., the average proportion of rescue free bowel movements per subject weighing less than 86 kg within 4 hours of all doses during the first 4 weeks of the study as set forth in Example 1.

FIG. 14 (Table 10) provides data related to the primary efficacy endpoint specific for subjects weighing 86 kg or more, i.e., the average proportion of rescue free bowel movements per subject weighing 86 kg or more within 4 hours of all doses during the first 4 weeks of the study as set forth in Example 1.

FIG. 15 (Table 11) provides data related to the primary efficacy endpoint specific for subjects having less than 3 rescue free bowel movements per week, i.e., the average proportion of rescue free bowel movements per subject having less than 3 rescue free bowel movements per week within 4 hours of all doses during the first 4 weeks of the study as set forth in Example 1.

FIG. 16 (Table 12) provides data related to the primary efficacy endpoint specific for subjects having 3 or more rescue free bowel movements per week, i.e., the average proportion of rescue free bowel movements per subject having 3 or more rescue free bowel movements per week within 4 hours of all doses during the first 4 weeks of the study as set forth in Example 1.

FIG. 17 (Table 13) provides data related to the primary efficacy endpoint specific for subjects having a Bristol Stool Form Scale Score less than 3, i.e., the average proportion of rescue free bowel movements per subject having a Bristol Stool Form Scale Score less than 3 within 4 hours of all doses during the first 4 weeks of the study as set forth in Example 1.

FIG. 18 (Table 14) provides data related to a key secondary efficacy endpoint, i.e., the change in weekly number of rescue free bowel movements from baseline over the first 4 weeks of the study as set forth in Example 1.

FIG. 19 (Table 15) provides data related to another key secondary efficacy endpoint, i.e., the proportion of subject responding to study drug wherein responding is defined as having at least 3 rescue free bowel movements per week for each of the 4 weeks of the study with an increase of at least one rescue free bowel movement over baseline for at least 3 weeks of the first 4 weeks of the study as set forth in Example 1.

FIG. 20 (Table 16) provides data related to a secondary efficacy endpoint, i.e., the proportion of subjects with rescue free bowel movements within 4 hours of the first dose of study drug as set forth in Example 1.

FIG. 21 (Table 17) summarizes adverse events that occurred amongst all subjects as set forth in Example 1.

FIG. 22 (Table 18) summarizes serious adverse events by system organ class that occurred amongst all subjects as set forth in Example 1.

FIG. 23 (Table 19) summarizes adverse events by system organ class that occurred amongst all subjects as set forth in Example 1.

FIG. 24 (Table 20) summarizes clinically significant ECG results as set forth in Example 1.

FIG. 25 is a schematic of the metabolic pathway of methylnaltrexone (MNTX) in humans.

FIG. 26 is a plot showing the MNTX mean plasma concentration vs. time profile following single oral 450 mg (3×150 mg) tablet dosed under fasted and fed conditions.

FIG. 27 is a plot showing the mean MNTX plasma concentration vs. time profile following single oral 150 mg, 300 mg or 450 mg tablet doses and a single subcutaneous 12 mg injection dose. The pharmacokinetic population is presented on semilogarithmic scale.

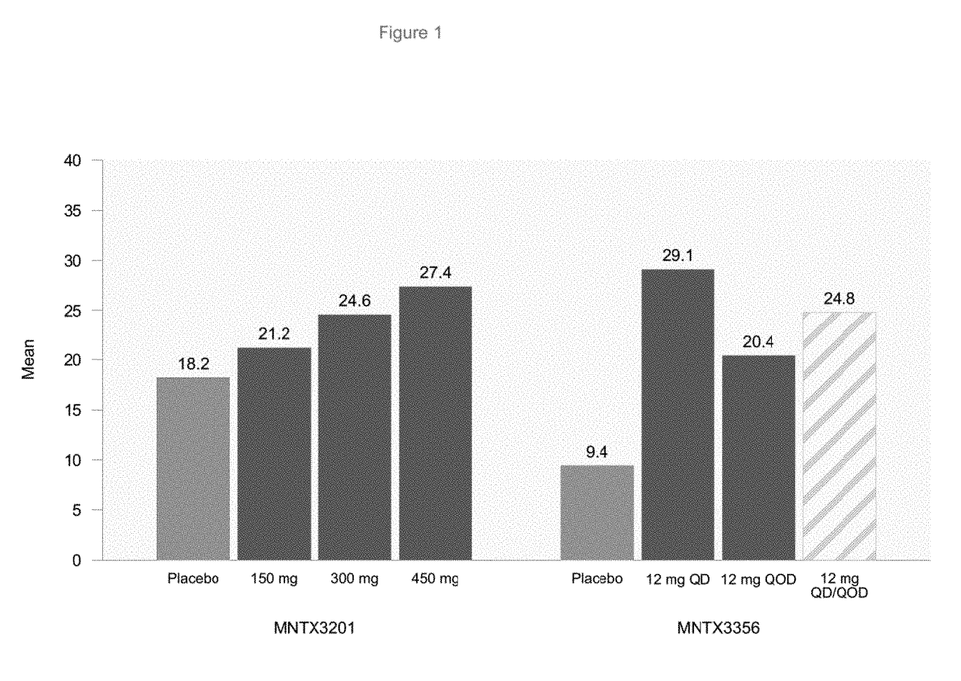

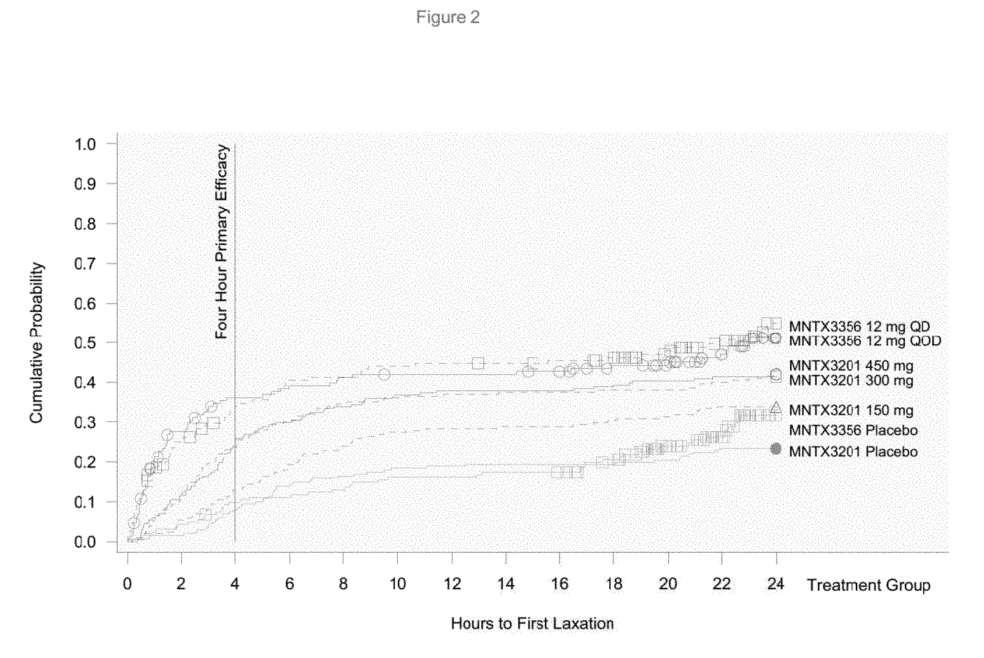

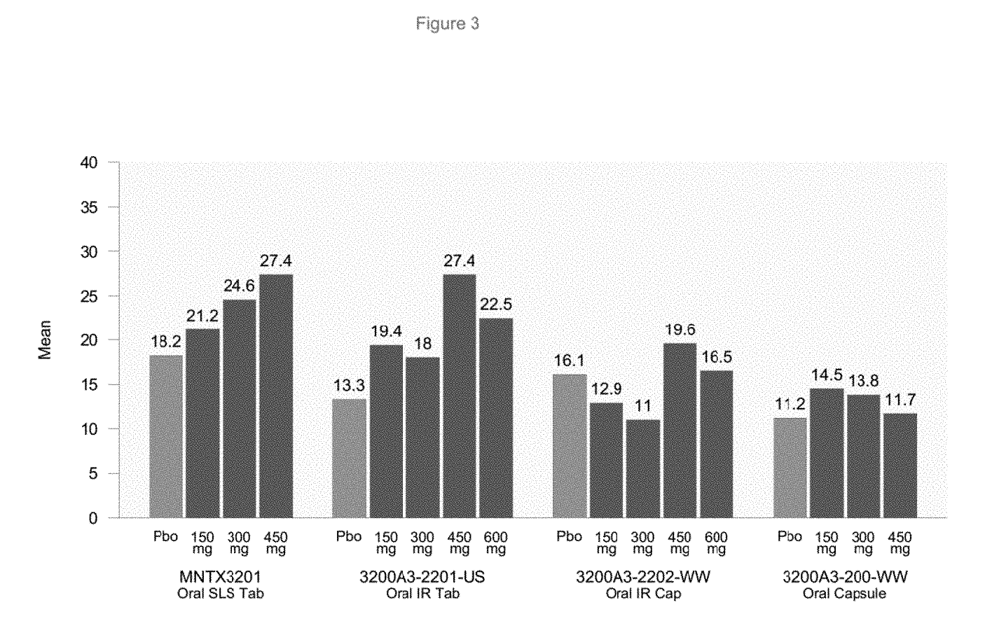

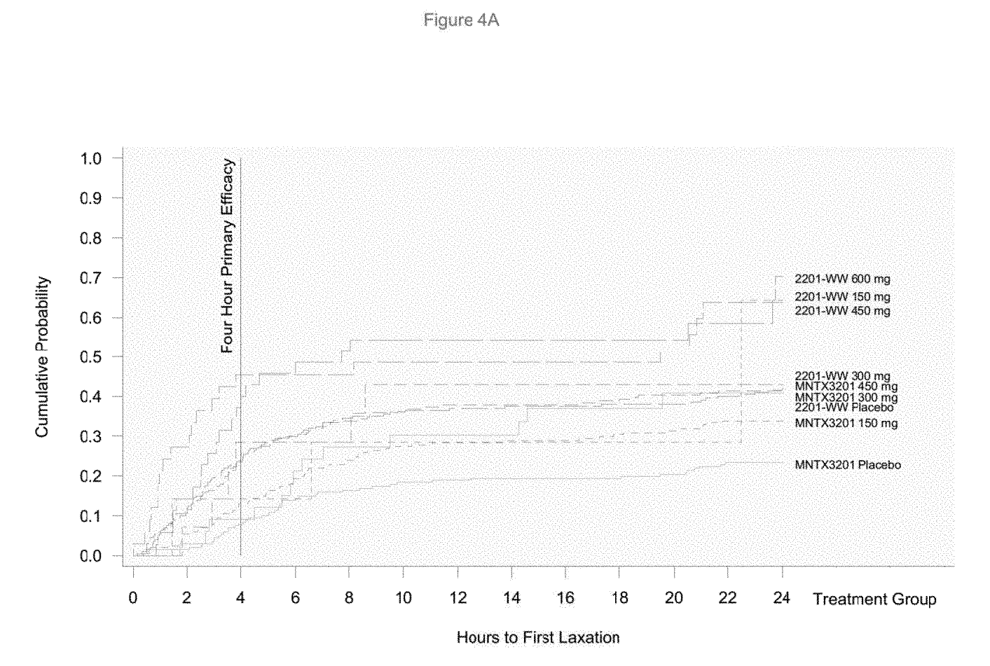

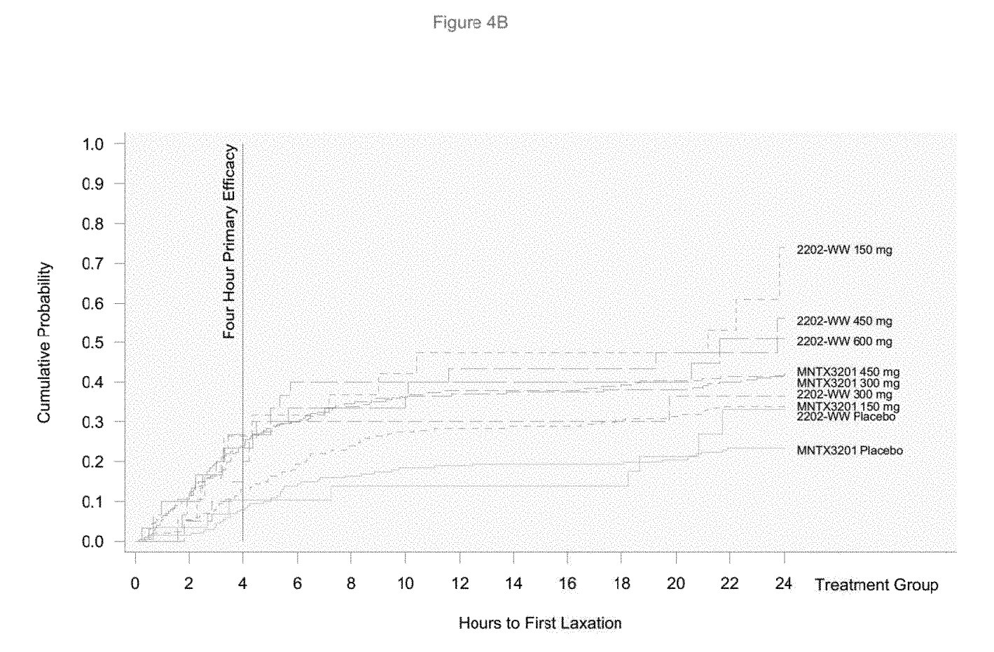

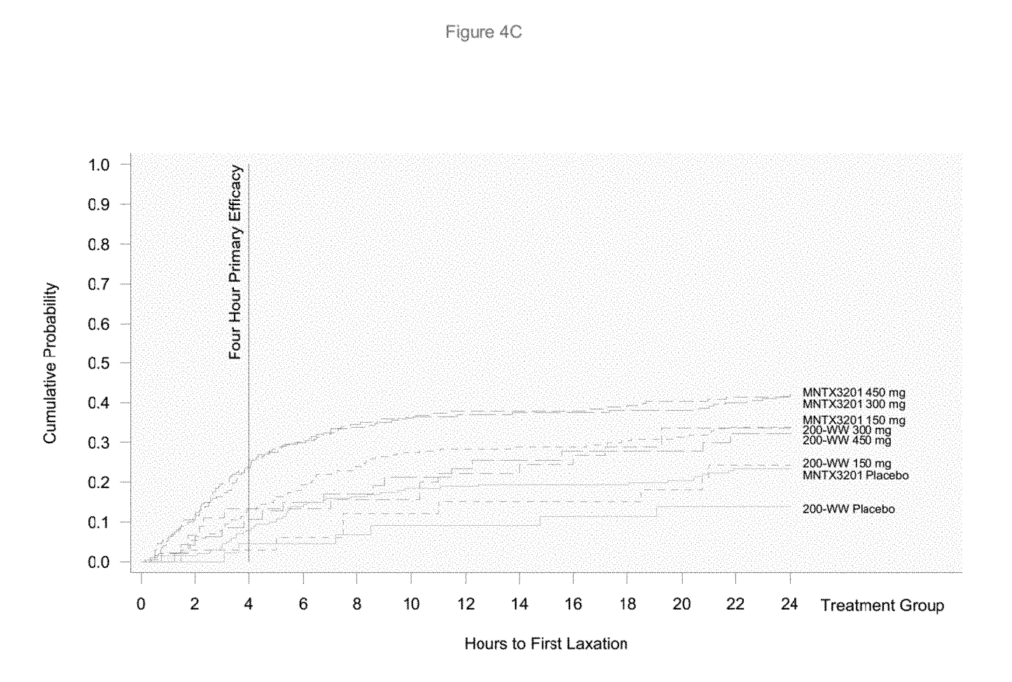

### DETAILED DESCRIPTION OF CERTAIN EMBODIMENTS OF THE INVENTION

Presented herein is the identification of methods for treatment of opioid induced constipation by administration of oral formulations of methylnaltrexone, for example, formulations including salts of methylnaltrexone including an anion of an amphiphilic pharmaceutically acceptable excipient. Moreover, presented herein is the identification that the daily oral administration of 150 mg, 300 mg or 450 mg of methylnaltrexone, for example, a composition comprising methylnaltrexone bromide and sodium dodecyl (lauryl) sulfate, is efficacious in treating or preventing opioid induced constipation without eliciting adverse events in the subject.

Unless otherwise defined herein, scientific and technical terms used herein shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including,” as well as other forms of the term, such as “includes” and “included”, is not limiting.

### Definitions

The term “constipation” as used herein, refers to a condition in which a subject suffers from infrequent bowel movements or bowel movements that are painful and/or hard to pass. A subject experiencing constipation often suffers from straining during bowel movements and/or a sensation of incomplete evacuation following bowel movements. In a particular embodiment, constipation refers to a subject who experiences less than three (3) rescue free bowel movements (RFBMs) per week on average, wherein “rescue free bowel movement” refers to the passage and evacuation of feces, or laxation.

As used herein, the term “opioid induced constipation” (OIC) refers to a subject who suffers from constipation resulting from opioid therapy. For example, a subject may suffer from opioid induced constipation arising from opioid therapy with alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and/or tramadol.

As used herein, an “effective amount” of an oral composition of methylnaltrexone refers to the level required to treat or prevent on or more symptoms of opioid induced constipation. In some embodiments, an “effective amount” is at least a minimal amount of an oral composition of methylnaltrexone, which is sufficient for treating or preventing one or more symptoms of opioid induced constipation, as defined herein. In some embodiments, the term “effective amount,” as used in connection with an amount of methylnaltrexone, salt thereof, or composition of methylnaltrexone or salt thereof, refers to an amount of methylnaltrexone, salt thereof, or composition of methylnaltrexone or salt thereof sufficient to achieve a rescue free bowel movement in a subject.

The terms “treat” or “treating,” as used herein, refers to partially or completely alleviating, inhibiting, delaying onset of, reducing the incidence of, ameliorating and/or relieving opioid induced constipation, or one or more symptoms of opioid induced constipation.

The expression “unit dosage form” as used herein refers to a physically discrete unit of a composition or formulation of methylnaltrexone, appropriate for the subject to be treated. It will be understood, however, that the total daily usage of provided formulation will be decided by the attending physician within the scope of sound medical judgment. The specific effective dose level for any particular subject will depend upon a variety of factors including the severity of the opioid induced constipation; nature and activity of the composition; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

As used herein, the term “non-malignant pain” refers to pain originating from a non-malignant source such as cancer.

The term “subject”, as used herein, means a mammal and includes human and animal subjects, such as domesticated animals (e.g., horses, dogs, cats, etc.) and experimental animals (e.g., mice, rats, dogs, chimpanzees, apes, etc.). In a particular embodiment, the subject is human.

The terms “suffer” or “suffering” as used herein refers to one or more conditions that a patient has been diagnosed with, or is suspected to have, in particular, opioid induced constipation.

The term “amphiphilic” as used herein to describe a molecule refers to the molecule's dual hydrophobic and hydrophilic properties. Typically, amphiphilic molecules have a polar, water soluble group (e.g., a phosphate, carboxylic acid, sulfate) attached to a nonpolar, water-insoluble group (e.g., a hydrocarbon). The term amphiphilic is synonymous with amphipathic. Examples of amphiphilic molecules include sodium dodecyl (lauryl) sulfate, fatty acids, phospholipids, and bile acids. Amphiphilic molecules may be uncharged, cationic, or anionic.

As used herein, the term “liphophilicity” refers to a compound's ability to associate with or dissolve in a fat, lipid, oil, or non-polar solvent. Lipophilicity and hydrophobicity may be used to describe the same tendency of a molecule to dissolve in fats, oils, lipids, and non-polar solvents.

### Compositions of Methylnaltrexone

In particular embodiments, the methods presented herein involve administration of oral compositions of methylnaltrexone comprising ion pairs of methylnaltrexone and an amphiphilic pharmaceutically acceptable excipient. For example, the composition for use in the methods presented herein may be a salt of methylnaltrexone of the formula:

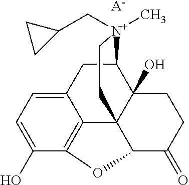

wherein methylnaltrexone is the cation of the salt, and A⁻ is an anion of an amphiphilic pharmaceutically acceptable excipient, as described in International Publication No. WO2011/112816, the entire contents of which are hereby incorporated by reference herein. In certain embodiments, the methylnaltrexone is (R)—N-methylnaltrexone, a peripherally acting μ opioid receptor antagonist, as shown in the formula above. It will be understood that the (R)—N-methylnaltrexone cation and the anion of the amphiphilic pharmaceutically acceptable excipient may exist in the composition as an ion pair or may exist as separate salts paired with other counter ions such as bromide and sodium, or mixtures thereof.

The compositions for oral administration further include an anion of an amphiphilic pharmaceutically acceptable excipient (A⁻). The amphiphlic pharmaceutically acceptable excipient increases the lipophilicity of the composition thereby allowing for increased transport through the unstirred diffusion layer in the GI tract, resulting in increased permeation through biological membranes. In certain embodiments, the excipient increases the lipophilicity of the drug.

In certain embodiments, the amphiphilic pharmaceutically acceptable excipient may include a sulfate, sulfonate, nitrate, nitrite, phosphate, or phosphonate moiety. In one embodiment, the pharmaceutically acceptable excipient comprises an (—OSO₃⁻) group. In certain embodiments, the anion is butyl sulfate, pentyl sulfate, hexyl sulfate, heptyl sulfate, octyl sulfate, nonyl sulfate, decyl sulfate, undecyl sulfate, dodecyl sulfate, tridecyl sulphate, tetradecyl sulfate, pentadecyl sulfate, hexadecyl sulfate, heptadecyl sulfate, octadecyl sulfate, eicosyl sulfate, docosyl sulfate, tetracosyl sulfate, hexacosyl sulfate, octacosyl sulfate, and triacontyl sulphate.

In certain embodiments, A⁻ is the anion of a Brønsted acid. Exemplary Brønsted acids include hydrogen halides, carboxylic acids, sulfonic acids, sulfuric acid, and phosphoric acid. In certain embodiments, A⁻ is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A⁻ is trifluoroacetate.

In certain embodiments, the methylnaltrexone in the composition may have multiple anions (e.g., bromide and dodecyl (lauryl) sulfate) associating therewith.

In certain embodiments, A⁻ is bromide, such that the compositions, and formulations thereof, comprise (R)—N-methylnaltrexone bromide. (R)—N-methylnaltrexone bromide, which is also known as “MNTX” and is described in international PCT patent application publication number, WO2006/12789, which is incorporated herein by reference. The chemical name for (R)—N-methylnaltrexone bromide is (R)—N-(cyclopropylmethyl) noroxymorphone methobromide. (R)—N-methylnaltrexone bromide has the molecular formula C₂₁H₂₆NO₄Br and a molecular weight of 436.36 g/mol. (R)—N-methylnaltrexone bromide has the following structure:

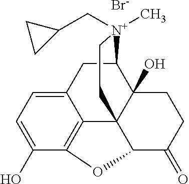 [SMILES: [Br-] ; C[N+]1(CC2CC2)CC[C@]23c4c5ccc(O)c4O[C@H]2C(=O)CC[C@@]3(O)[C@H]1C5]

### (R)—N-methylnaltrexone bromide

where the compound is in the (R) configuration with respect to the quaternary nitrogen. In certain embodiments presented herein, at least about 99.6%, 99.7%, 99.8%, 99.85%, 99.9%, or 99.95% of the compound is in the (R) configuration with respect to nitrogen. Methods for determining the amount of (R)—N-methylnaltrexone bromide, present in a sample as compared to the amount of (S)—N-methylnaltrexone bromide present in that same sample, are described in detail in WO2006/127899, which is incorporated herein by reference. In other embodiments, the methylnaltrexone contains 0.15%, 0.10%, or less (S)—N-methylnaltrexone bromide.

In certain embodiments, A⁻ is an acidic amphiphilic pharmaceutically acceptable excipient. In certain embodiments, the pharmaceutically acceptable excipient has a pKₐ of about 3 or less. In certain embodiments, the pharmaceutically acceptable excipient has a pKₐ of about 2 or less. In certain embodiments, the pharmaceutically acceptable excipient has a pKₐ between about 1 and about 2. In certain embodiments, the pharmaceutically acceptable excipient has a pKₐ of about 1 or less.

In some embodiments, the compositions for oral administration are tablet formulations. In some embodiments, the compositions for oral administration are capsule formulations. Methylnaltrexone for use in such compositions and formulations may be in any of a variety of forms. For example, forms of methylnaltrexone suitable for use in the inventive compositions and formulations include pharmaceutically acceptable salts, prodrugs, polymorphs (i.e., crystal forms), co-crystals, hydrates, solvates, and the like. Any form of methylnaltrexone may be used in the compositions or formulations, but the form should allow for ion pairing with the amphiphilic pharmaceutically acceptable excipient. In certain embodiments, the methylnaltrexone ion pair is a salt that is solid at room temperature. In some embodiments, the composition is a pharmaceutical composition.

In general, formulations for oral administration comprise methylnaltrexone, an amphiphilic pharmaceutically acceptable excipient as described above, and a disintegrant, and further, optionally, comprise one or more other components, such as, for example, binders, carriers, chelating agents, antioxidants, fillers, lubricants, wetting agents, or combinations thereof, as set forth in International Publication No. WO2011/112816, the entire contents of which are hereby incorporated by reference herein.

In a particular embodiment, the composition, for example, pharmaceutical composition, for oral administration comprises methylnaltrexone bromide and sodium dodecyl (lauryl) sulfate (also known as SDS or SLS). In certain embodiments, the composition further includes sodium bicarbonate as a disintegrant. Additional excipients, as set forth above, may be incorporated, including, but not limited to, at least one of microcrystalline cellulose, crospovidone, polysorbate 80, edetate calcium disodium dehydrate, silicified microcrystalline cellulose, talc, colloidal silicon dioxide and magnesium stearate. In one embodiment, the composition for oral administration comprises each of methylnaltrexone bromide, sodium lauryl sulfate, sodium bicarbonate, microcrystalline cellulose, crospovidone, polysorbate 80, edetate calcium disodium dehydrate, silicified microcrystalline cellulose, talc, colloidal silicon dioxide and magnesium stearate.

Compositions and formulations thereof for use as described herein may be generated as set forth in International Publication No. WO2011/112816, the entire contents of which are hereby incorporated by reference herein. Additionally, compositions, and formulations thereof, may be generated as described in Examples 2-4 herein.

### Selection of Subjects for Treatment

In certain aspects, the selection of certain subjects suffering from opioid induced constipation for treatment with oral compositions of methylnaltrexone and subsequent administration of the oral compositions is presented herein.

As defined herein, a subject suffering from opioid induced constipation refers to a subject who suffers from constipation resulting from opioid activity, for example, exogenous opioid therapy or endogenous opioid activity. “Constipation” refers to a condition in which a subject suffers from infrequent bowel movements or bowel movements that are painful and/or hard to pass. A subject experiencing constipation often suffers from hard or lumpy stools, straining during bowel movements and/or a sensation of incomplete evacuation following bowel movements. In a particular embodiment, constipation refers to a subject who experiences less than three (3) rescue free bowel movements (RFBMs) per week on average, for example, over the course of the last four consecutive weeks, wherein “rescue free bowel movement” refers to the passage and evacuation of feces, or laxation.

In certain embodiments, the subject does not have a history of chronic constipation prior to the initiation of opioid therapy.

Subjects who are on opioid therapy, who have recently been on opioid therapy or who intend to be on opioid therapy, may be administered the oral compositions of methylnaltrexone. In one embodiment, the subject, at the time of the screening, is on an opioid therapeutic regimen and has been on such regimen for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days. In a particular embodiment, the subject has been taking opioids for at least one month. In another embodiment, the subject, at the time of the screening, will begin an opioid therapeutic regimen at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days after the screening. In yet another embodiment, the subject, at the time of the screening, will have discontinued opioid therapeutic regimen less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days prior to the screening.

The subject may be on an opioid regimen for a variety of purposes. For example, the subject may be a cancer or surgical patient, an immunosuppressed or immunocompromised patient (including HIV infected patient), a patient with advanced medical illness, a terminally ill patient, a patient with neuropathies, a patient with rheumatoid arthritis, a patient with osteoarthritis, a patient with chronic pack pain, a patient with spinal cord injury, a patient with chronic abdominal pain, a patient with chronic pancreatic pain, a patient with pelvic perineal pain, a patient with fibromyalgia, a patient with chronic fatigue syndrome, a patient with migraine or tension headaches, a patient on hemodialysis, or a patient with sickle cell anemia.

In various embodiments, the subject is receiving opioids for alleviation of pain. In a particular embodiment, the subject is receiving opioids for alleviation of chronic non-malignant pain. As used herein, the term “non-malignant pain” refers to pain originating from a non-malignant source such as cancer. In particular embodiments, non-malignant pain includes to back pain, cervical pain, neck pain, fibromyalgia, low extremity pain, hip pain, migraines, headaches, neuropathic pain, or osteoarthritis.

As used herein, the term “chronic” refers to a condition that persists for an extended period of time. In various embodiments, chronic may refer to a condition that lasts at least 1, 2, 3 or 4 weeks. Alternatively, chronic may refer to a condition that lasts at least 1, 2, 3, 4, 5, 6, 7, 8, 9, 10, 11, 12, 18, 24, 30 or 36 months. In a particular embodiment, the subject is receiving opioids for alleviation of chronic non-malignant pain that has persisted for at least 2 months.

In various embodiments, the subject may be on opioid therapy including, but not limited to, alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and/or tramadol.

In various embodiments, the subject is receiving a daily dose of at least 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of oral morphine equivalents. In a particular embodiment, the subject is receiving at least 50 mg of oral morphine equivalents. Calculation of oral morphine equivalents is well known in the art. Table A provides a morphine oral equivalence table for known opioids.

TABLE A
Morphine Oral Equivalence Table

| | | | Factor for |
|---|---|---|---|
| | | | Morphine |
| | | | Equivalents |
| Drug | Route | Units | in mgs |
| ALFENTANIL | IV | meg | 0.6 |
| CODEINE | PO | mg | 0.3 |
| CODEINE CONTIN | PO | mg | 0.3 |
| FIORICET WITH CODEINE CAPSULES | PO | mg | 0.3 |
| PANADEINE FORTE | PO | mg | 0.3 |
| PHENERGAN WITH CODEINE | PO | mg | 0.3 |
| TYLENOL W/CODEINE NO. 2 | PO | mg | 0.3 |
| TYLENOL W/CODEINE NO. 3 | PO | mg | 0.3 |
| TYLENOL WITH CODEINE | PO | mg | 0.3 |
| DEMEROL | IM | mg | 1.25 |
| DEMEROL | IV | mg | 1.25 |
| DEMEROL | PO | mg | 0.2 |
| DURAGESIC | TD | meg/hr | 3.6 |
| FENTANYL | IV | meg | 0.6 |
| FENTANYL | IV | mg | 600 |
| FENTANYL | PO | meg | 0.076 |
| FENTANYL CITRATE | PO | mg | 75 |
| FENTANYL CITRATE | PO | meg | 0.076 |
| FENTANYL | TD | meg/hr | 3.6 |
| ACETAMINOPHEN W/ | PO | mg | 1.8 |
| HYDROCODONE BITARTRATE | | | |
| APAP WITH HYDROCODONE | PO | mg | 1.8 |
| HYCODAN | PO | mg | 1.8 |
| HYDROCODONE | PO | mg | 1.8 |
| LORCET | PO | mg | 1.8 |
| LORTAB | PO | mg | 1.8 |
| TUSSIONEX | PO | mg | 1.8 |
| VICODIN | PO | mg | 1.8 |
| VICODIN ES | PO | mg | 1.8 |
| VICOPROFEN | PO | mg | 1.8 |
| ZYDONE | PO | mg | 1.8 |
| DILAUDID | IV | mg | 40 |
| DILAUDID | PO | mg | 8 |
| HYDROMORPH CONTIN | PO | mg | 8 |
| HYDROMORPHONE | PO | mg | 8 |
| HYDROMORPHONE | PO | mg | 8 |
| HYDROCHLORIDE | | | |
| METHADONE | PO | mg | 3 |
| METHADONE HYDROCHLORIDE | PO | mg | 3 |
| METHADOSE | PO | mg | 3 |
| MORPHINE | IV | mg | 6 |
| MORPHINE | PO | mg | 1 |
| MORPHINE HYDROCHLORIDE | PO | mg | 1 |
| MORPHINE SULFATE | PO | mg | 1 |
| MS CONTIN | PO | mg | 1 |
| MSIR | PO | mg | 1 |
| MSIR | PR | mg | 1 |
| ORAMORPH | PO | mg | 1 |
| STATEX | PO | mg | 1 |
| ACETAMINOPHEN W/OXYCODONE | PO | mg | 2 |
| ENDONE | PO | mg | 2 |
| OXYCOCET | PO | mg | 2 |
| OXYCODONE | PO | mg | 2 |
| OXYCODONE HYDROCHLORIDE | PO | mg | 2 |
| PERCOCET | PO | mg | 2 |
| SUPEUDOL | PO | mg | 2 |
| TYLOX | PO | mg | 2 |
| OXYMORPHONE | IV | mg | 60 |
| OXYMORPHONE | PO | mg | 3 |
| OXYMORPHONE HYDROCHLORIDE | PO | mg | 3 |
| DARVOCET | PO | mg | 0.234 |
| DARVOCET-N | PO | mg | 0.15 |
| DARVON | PO | mg | 0.234 |
| DARVON-N | PO | mg | 0.15 |
| PROPOXYPHENE | PO | mg | 0.234 |
| REMIFENTANIL | IV | meg | 0.6 |
| ROXICET | PO | mg | 2 |
| SUFENTANIL | IV | mg | 6000 |
| SUFENTANIL | IV | meg | 6 |
| TRAMADOL | PO | mg | 0.2 |
| TRAMADOL HYDROCHLORIDE | PO | mg | 0.2 |
| TRAMAL | PO | mg | 0.2 |
| ULTRACET | PO | mg | 0.2 |
| TAPENTADOL | PO | mg | 0.33 |

Foley K M. The treatment of cancer pain. N Engl J Med. 1985 July, 313(2): 84-95

The subject's opioid therapeutic regimen may be by any mode of administration. For example, the subject may be taking opioids orally, transdermally, intravenously, or subcutaneously.

### Dosage and Administration

Compositions and formulations may be administered to a patient as required to provide an effective amount of methylnaltrexone. As defined above, an “effective amount” of a compound or pharmaceutically acceptable composition can achieve a desired therapeutic and/or prophylactic effect. In some embodiments, an “effective amount” is at least a minimal amount of a compound, or composition containing a compound, which is sufficient for treating or preventing one or more symptoms of opioid induced constipation, as defined herein. In some embodiments, the term “effective amount,” as used in connection with an amount of methylnaltrexone, salt thereof, or composition of methylnaltrexone or salt thereof, refers to an amount of methylnaltrexone, salt thereof, or composition of methylnaltrexone or salt thereof sufficient to achieve a rescue free bowel movement in a subject.

In some embodiments, the oral composition of methylnaltrexone is sufficient to achieve a rescue free bowel movement in a subject within about 24 hours, within about 12 hours, within about 8 hours, within about 5 hours, within about 4 hours, within about 3 hours, within about 2 hours, or within about 1 hours of administration to said patient. In a particular embodiment, the oral composition of methylnaltrexone is sufficient to achieve a rescue free bowel movement within about 4 hours of administration to the patient. In some embodiments, the oral composition of methylnaltrexone is sufficient to achieve a rescue free bowel movement within about 4 hours of administration to the patient for at least 100%, 99%, at least 95%, at least 90%, at least 85%, at least 80%, at least 75%, or at least 50% of all doses administered. In certain embodiments, the oral composition of methylnaltrexone is sufficient to achieve a rescue free bowel movement within four hours during the first 1, 2, 3, 4, 5, 6, 7, 8, 9 or 10 weeks of dosing. In a particular embodiment, the oral composition of methylnaltrexone is sufficient to achieve a rescue free bowel movement within about 4 hours of administration to the patient for all doses administered during first four weeks of dosing.

The efficacy of the oral compositions presented herein in treating opioid induced constipation may further be assessed by an increase in the number of rescue free bowel movements experienced by a subject. For example, in some embodiments, the oral composition of methylnaltrexone is sufficient to increase the weekly number of rescue free bowel movements experienced by a subject by at least 1, 2, 3, 4, 5, 6, 7, 8, 9 or 10. In particular embodiments, the oral composition of methylnaltrexone is sufficient to increase the weekly number of rescue free bowel movements experienced by a subject by at least 1. In another embodiment, the oral composition of methylnaltrexone is sufficient to increase the weekly number of rescue free bowel movements experienced by a subject by at least 2. In yet another embodiment, the oral composition of methylnaltrexone is sufficient to increase the weekly number of rescue free bowel movements experienced by a subject by at least 3. In certain embodiments, the oral composition of methylnaltrexone is sufficient to increase the weekly number of rescue free bowel movements experienced by a subject during the first 1, 2, 3, 4, 5, 6, 7, 8, 9 or 10 weeks of dosing. In a particular embodiment, the oral composition of methylnaltrexone is sufficient to increase the weekly number of rescue free bowel movements experienced by a subject by at least 1 during the first 4 weeks of dosing. In another particular embodiment, the oral composition of methylnaltrexone is sufficient to increase the weekly number of rescue free bowel movements by at least one to at least 3 a week. In yet a further embodiment, the oral composition of methylnaltrexone is sufficient to increase the weekly number of rescue free bowel movements by at least one to at least 3 a week for at least 3 of the first 4 weeks following administration.

The efficacy of the oral compositions presented herein may be further assessed using various assessment tools available to those skilled in the art to assess treatment of constipation.

In a particular embodiment, the efficacy of the oral compositions of methylnaltrexone is assessed by Patient Assessment of Constipation (PAC) questionnaires. The PAC consists of two complementary questionnaires: the PAC-Symptoms (SYM) and the PAC-Quality of Life (QoL) questionnaires. The PAC-SYM is a 12 item survey that measures the severity of constipation symptoms across three domains: stool symptoms, rectal symptoms and abdominal symptoms. The PAC-SYM scale has been used primarily to evaluate chronic constipation. The PAC-SYM scale is further described in Frank et al. Scand J Gastroenterol (1999) 34(9):870-877 and Slappendel et al. European Journal of Pain (2006) 10(3):209-217, the entire contents of each of which are incorporated by reference herein. The PAC-QoL is a 28-item survey that measures constipation-specific quality of life across four domains: worries and concerns, physical discomfort, psychosocial discomfort, and satisfaction. The PAC-QoL scale is further described in Marquis et al. SJG (2005) 40:540-551, the entire contents of which are incorporated by reference herein.

Alternatively or in combination, the efficacy of the oral compositions of methylnaltrexone is assessed by the European Quality of Life-5 Dimensions (EQ-5D) analysis. The EQ-5D is a 5-item standardized instrument for use as a measure of patient reported outcome (PRO). Applicable to a wide range of health conditions and treatments, the instrument provides a simple descriptive profile and a single index value for health status. The EQ-5D instrument is further described in Dolan P. Medical Care (1997) 35:1095-1108, Rabin R. Ann. Med. (2001) 33(5):537-543 and Shaw et al. Medical Care (2005) 43:203-220, the entire contents of each of which are incorporated by reference herein.

Alternatively or in combination, the efficacy of the oral compositions of methylnaltrexone is assessed by the Work Productivity and Activity Impairment General Health V2.0 (WPAI:GH) questionnaire. The WPAI:GH is a 6-item questionnaire to quantify lost time from work and loss in productivity for health problems. The WPAI:GH yields 4 types of scores: absenteeism (work time missed), “presenteeism” (impairment at work/reduced on-the-job effectiveness), work productivity loss (overall work impairment/absenteeism plus presenteeism), and activity impairment. The WPAI:GH questionnaire is further described in Reilly et al. PharmacoEconomics (1993) 4(51:353-365, the entire contents of which are incorporated by reference herein.

Alternatively or in combination, the efficacy of the oral compositions of methylnaltrexone is assessed by the Global Clinical Impression of Change (GCIC) scale. The GCIC is a 7 point rating scale designed to assess subject's and clinician's impression of the subject's change in bowel status while on study drug. The scale ranges from 1 (Much Worse) to 7 (Much Better). This scale was completed by the subject and clinician at the end of daily dosing and End of Treatment.

In certain embodiments, the patient is orally administered a composition of methylnaltrexone at least once a day. In certain embodiments, the subject is administered an oral composition of methylnaltrexone at least once, twice, three, four or five times a day. In a particular embodiment, the subject is administered an oral composition of methylnaltrexone three times a day.

In various embodiments, the subject is orally administered 150 mg of methylnaltrexone, or a salt thereof, daily. For example, the subject may be administered a tablet comprising 150 mg of methylnaltrexone or a salt thereof, daily. In another embodiment, the subject is orally administered 300 mg of methylnaltrexone or a salt thereof, daily. For example, the subject may be administered two tablets, each comprising 150 mg of methylnaltrexone or a salt thereof, daily. In yet another embodiment, the subject is orally administered 450 mg of methylnaltrexone or a salt thereof, daily. For example, the subject may be administered three tablets, each comprising 150 mg of methylnaltrexone or a salt thereof, daily.

### Adverse Events

Presented herein are methods that may be predicated, at least in part, on the identification that administration of oral compositions of methylnaltrexone, for example, 150 mg, 300 mg or 450 mg, at least once a day, for example, three times a day, is sufficient to treat opioid induced constipation without effecting adverse events. Exemplary adverse events induced by the administering oral methylnaltrexone are set forth in example 1. The invention also provides methods of treating a subject with oral formulations of methylnaltrexone described herein that decrease the occurrence of adverse events in comparison to the frequency of adverse events observed with previous oral methylnaltrexone formulations, for example, enterically coated oral formulations of methylnaltrexone or other oral formulations of methylnaltrexone not including an anion of an amphiphilic pharmaceutically acceptable excipient, in particular, sodium dodecyl (lauryl) sulfate.

Accordingly, the data presented in Example 1 demonstrate that the methods of administering the oral formulations of methylnaltrexone described herein are safer than the methods of administering previously described oral formulations of methylnaltrexone, for example, enterically coated oral formulations of methylnaltrexone or other oral formulations of methylnaltrexone not including an anion of an amphiphilic pharmaceutically acceptable excipient, in particular, sodium dodecyl (lauryl) sulfate.

All features of each of the aspects presented herein apply to all other aspects mutatis mutandis. The contents of all references, patents, pending patent applications and published patents, cited throughout this application are hereby expressly incorporated by reference.

### EXAMPLES

### Example 1: Efficacy and Dosage Studies of Oral Methylnaltrexone in Treatment of Opioid Induced Constipation

### Objectives

### Primary Objective

The primary objective of this study was to evaluate the safety and efficacy of Oral Methylnaltrexone (OM) versus placebo in subjects with chronic non-malignant pain who have Opioid Induced Constipation (OIC).

### Secondary Objectives

The secondary objective of this study was to determine OM dosing regimen in subjects with chronic non-malignant pain who have OIC.

### Study Design

A phase 3, multicenter, randomized, double-blind, placebo-controlled, parallel-group study of OM for the treatment of OIC in approximately 802 subjects with chronic non-malignant pain was conducted.

Eligible subjects signed an informed consent form (ICF) and entered a 14-day screening period (±2 days), during which objective evidence of constipation was assessed and used as the basis for enrollment.

Constipation due to opioid use during the screening period: Constipation is defined as <3 Rescue-Free Bowel Movements (RFBMs) per week on average (no laxative use within 24 hours prior to bowel movement) that were associated with 1 or more of the following (based on subject's diary report):
a. A Bristol Stool Form Scale type 1 or 2 for at least 25% of the rescue-free bowel movements.
b. Straining during at least 25% of the rescue-free bowel movements.
c. A sensation of incomplete evacuation after at least 25% of the rescue-free bowel movements.

Subjects who remained eligible at the baseline visit (day 1) were randomly assigned to either OM tablet formulation 150 mg, 300 mg, 450 mg, or placebo initially in a 1:1:1:1 allocation ratio. Subjects were required to take three tablets per day, first thing in the morning on an empty stomach (prior to breakfast). Subjects were instructed to swallow the tablets whole and never to chew, divide, or crush them and wait at least one half hour before ingesting any food. Subjects participated in the study for up to 84 days. The first 28 days were once daily dosing; the remaining 56 days were dosing as needed (PRN). Dosing remained double-blind throughout the 12 week period (84 days). The 84 day treatment period were followed by a 14-day post-treatment follow-up period (±2 days). Enrollment continued until a total of approximately 802 subjects have been randomized and dosed.

### Study Conduct

The study was divided into a screening period (14 days in duration [±2 days]), a doubleblind daily dosing period (28 days in duration), a double blind PRN dosing period (56 days in duration), and follow-up visit (14-day post-treatment follow-up visit [±2 days]).

a. Study Conduct—Screening Period

The screening period was a 14 day period (±2 days) prior to dosing. Upon receipt of their signed and dated written ICF, subjects had their eligibility status assessed prior to participation in the study. Screen failure, for the purpose of this study, was defined as any subject who signed an informed consent form but did not receive any study drug. All laxative therapy was discontinued at the start of the screening and only study-permitted rescue laxatives were used throughout the screening and double-blind periods.

b. Study Conduct—Double Blind Period

At the baseline visit, subjects were randomly assigned to either OM or placebo. Subjects who met all inclusion and no exclusion criteria at the baseline visit (day 1) received study medication. All doses were to be taken in the morning prior to breakfast [The first dose administered at the baseline visit could have been taken after Noon (12:00 pm)] and subjects were instructed to wait at least one half hour before ingesting any food. Subjects participated in the study for up to 84 days: the first 28 days were double-blind once daily dosing; the remaining 56 days were double-blind PRN dosing.

c. Study Conduct—End of Treatment

When a subject completed or discontinued from the study, all evaluations were conducted at day 84 or at an early termination visit. This evaluation included the following: a vital sign measurement, specimen collection for laboratory determinations, physical exam, serum pregnancy test (if applicable), recording and reconciliation of AEs, concomitant opioids, nonopioid treatments, OOWS, SOWS, Pain Intensity Scale, quality of life and constipation symptom assessments, Global Clinical Impression of Change (GCIC), and review of subject reported diary information and compliance.

d. Study Conduct—Follow Up Visit

Subjects, who completed the 12 week (84 day) double-blind phase, returned for a follow up visit 14 days (±2 days) after Day 84 to assess the subject's overall safety status.

### Investigational Plan—Overall Study Design and Rationale, Choice of Control Groups, and Appropriateness of Measurements

The primary efficacy endpoint of this Phase 3 study was the average proportion of rescue-free laxation responses per subject within 4 hours of all doses during the first four weeks of dosing. The key secondary efficacy endpoints in hierarchical order were:

1. Change in weekly number of RFBM from baseline during Weeks 1 to 4

2. Response (responder/non-responder) to study drug during Weeks 1 to 4, where responder was defined as having ≥3 RFBM/week, with at least 1 RFBM/week increase over baseline, for at least 3 out of the first 4 weeks.

### Choice of Treatment Groups

The active oral methylnaltrexone (OM) doses that were assessed included 150, 300, and 450 mg and were part of a placebo-controlled design to assess the safety and efficacy of OM. The placebo control design (allowed blinding, randomization and included a group that receives an inert treatment) controlled for potential influences other than those arising from the pharmacologic action of the study drug. These influences included safety findings associated with the underlying condition, spontaneous change (natural history of the condition and regression to the mean), subject or investigator expectations, the effect of being in a trial, use of other therapy, and subjective elements of diagnosis or assessment. For these reasons, the placebo-controlled design was ethically acceptable and consistent with the Declaration of Helsinki as clarified by the World Medical Association General Assembly, Washington, 2002.

### Study Criteria

Only subjects who met eligibility criteria were enrolled in the study.

Subjects were permitted to continue to be included in the study only if they met the inclusion criteria at the Baseline Visit.

Subjects were excluded from the study if they met any one of the exclusion criteria at the Screening Visit.

Subjects were excluded from the study if they met any one of the exclusion criteria at the Baseline Visit.

### Screening

An eligibility assessment to ensure the presence of required inclusion criteria and the absence of all exclusion criteria was performed and verified on the source and CRF. At the screening visit, subjects who were eligible for the study were asked to return for the day 1 visit.

### Assessment of Efficacy

To assess for efficacy, subject-reported information including date and time of bowel movements, Bristol Stool Form Scales, Straining Scales, Sense of Complete Evacuation Scales, and recording of study drug and rescue laxative use.used.

Primary Efficacy Endpoints

The primary efficacy endpoint was the average proportion of rescue-free laxation responses per subject within 4 hours of all doses during the first 4 weeks of dosing.

Secondary Efficacy Endpoints

The two key secondary efficacy endpoints in hierarchical order were:
1. Change in weekly number of RFBMs from baseline over the entire first 4 weeks (28 days) of dosing.
2. Response (responder/non-responder) to study drug during Weeks 1 to 4, where responder is defined as having >3 RFBM/week, with at least 1 RFBM/week increase over baseline, for at least 3 out of the first 4 weeks.

Other Secondary Efficacy Endpoints

Other endpoints included:
Proportion of subjects achieving at least 3 RFBMs per week
Proportion of subjects with rescue-free laxation response within 4 hours of the first dose of study drug by fasting status
Time to the first RFBM after the first dose, censored at 24 hours or time of the second dose, whichever occurred first by fasting status
Response (responder/non-responder) to study drug over the entire 12 week treatment period, where a responder is having ≥3 RFBM/week, with at least 1 RFBM/week increase over baseline, for ≥75% of the weeks
Percentage of doses resulting in any RFBM within 1, 2, 3, 4, 6, 8, and 24 hour(s)

### Assessment of Safety

Subjects were monitored for adverse events (AEs), serious adverse events (SAEs) concomitant treatments including opioid use and rescue laxatives, and vital sign measurements at all office visits. Vital signs, physical examinations (including rectal examination), laboratory evaluations, serum/urine pregnancy tests, ECGs, the Objective Opioid Withdrawal Scale (OOWS), the Subjective Opioid Withdrawal Scale (SOWS) and the Pain Intensity scale were performed at scheduled intervals during die study.

Electrocardiograms

Standard 12-lead ECGs were obtained after the subject had been resting for at least five minutes at the visits designated in the Schedule of Study Visits and Evaluations. The Investigator was responsible for reviewing, interpreting, and retaining hard copies of the reports. Clinically significant abnormalities at any time point after the normal or non-clinically significant screening ECG were recorded as adverse events, as defined below.

### Patient Reported Outcomes

Self-administered PRO endpoints were measured by the PAC-SYM, the PAC-QoL, the EQ-5D, the WPAI:GH, and GCIC (administered by the clinician) assessments quantify the subjects' constipation symptoms, constipation-related quality of life, overall quality of life, change in bowel status, and degree of interference with ability to work.

Pain Intensity Scale

Measures of pain were recorded using The Numerical Rating of Pain Intensity Scale. The scale, an 11-point rating scale ranging from 0 (None) to 10 (Worst Pain Possible), is a subject assessment tool and subjects should complete the evaluation based on their pain experienced during the 24 hours prior to completing the scale.

Bristol Stool Scale

Measures of stool consistency and straining were recorded for each bowel movement using the Bristol Stool Scale. The Bristol Stool Scale is a 7-point scale rating the characteristics of the stool sample. The range is from Type 1, Separate hard lumps, like nuts (hard to pass) to Type 7, Watery, no solid pieces, entirely liquid. The Bristol Stool Scale is a recognized, general measure of stool consistency or form.

Straining Scale

Measures of straining were recorded for each bowel movement using the Straining Scale. The scale, a five-point scale to rate the amount of straining (None to Very Severe), is a subject assessment tool and subjects were to complete the evaluations for each bowel movement.

Sense of Complete Evacuation Scale

Measures of the sense of complete evacuation were recorded for each bowel movement using the Sense of Complete Evacuation Scale. The scale is a subject assessment tool and subjects were to complete the evaluations for each bowel movement.

Patient Reported Outcomes (PROs)

The PROs are for the purpose of exploring the subject's experience of constipation symptoms and the impact of constipation on quality of life and work productivity. Every effort was to be made to maintain an unbiased assessment. The investigator was to not influence the subject's self-assessments.

Patient Assessment of Constipation (PAC):

The PAC consists of two complementary questionnaires: the PAC-Symptoms (SYM) and the PAC-Quality of Life (QoL). The PAC-SYM is a 12 item survey that measures the severity of constipation symptoms across three domains: stool symptoms, rectal symptoms and abdominal symptoms. The PAC-SYM scale has been use primarily to evaluate chronic constipation. The PAC-QoL is a 28-item survey that measures constipation-specific quality of life across four domains: worries and concerns, physical discomfort, psychosocial discomfort, and satisfaction.

European Quality of Life-5 Dimensions (EQ-5D):

The EQ-51) is a 5-item standardized instrument for use as a measure of PRO. Applicable to a wide range of health conditions and treatments, it provides a simple descriptive profile and a single index value for health status.

Work Productivity and Activity Impairment General Health V2.0 (WPAI:GH):

The WPAI:GH is a 6-item questionnaire to quantify lost time from work and loss in productivity for health problems. The WPAI:GH yields 4 types of scores: absenteeism (work time missed), “presenteeism” (impairment at work/reduced on-the-job effectiveness), work productivity loss (overall work impairment/absenteeism plus presenteeism) and activity impairment.

Global Clinical Impression of Change (GCIC):

The GCIC is a 7 point rating scale designed to assess subject's and clinician's impression of the subject's change in bowel status while on study drug. The scale ranges from 1 (Much Worse) to 7 (Much Better). This scale was completed by the subject and clinician at the end of daily dosing (Visit 4) and End of Treatment (Visit 7).

Study drug was provided in blister cards containing 150 mg tablets of active study drug and/or placebo. Each card had 21 study drug tablets, which is seven days worth of study medication. Three tablets will be taken at a time.

### Data Analysis

Endpoints and Assessments

Primary:

Average proportion of rescue-free laxation responses per subject within 4 hours of all doses during the first 4 weeks of dosing

Secondary:
1. Change in weekly number of RFBMs from baseline over the entire first 4 weeks (28 days) of dosing.
2. Response (responder/non-responder) to study drug during Weeks 1 to 4, where responder is defined as having ≥3 RFBM/week, with at least 1 RFBM/week increase over baseline, for at least 3 out of the first 4 weeks.

Other Secondary:
Proportion of subjects achieving at least 3 RFBMs per week
Proportion of subjects with rescue-free laxation response within 4 hours of the first dose of study drug by fasting status
Time to the first RFBM after the first dose, censored at 24 hours or time of the second dose, whichever occurs first by fasting status
Response (responder/non-responder) to study drug over the entire 12 week treatment period, where a responder is having ≥3 RFBM/week, with at least 1 RFBM/week increase over baseline, for ≥75% of the weeks
Percentage of doses resulting in any RFBM within 1, 2, 3, 4, 6, 8, and 24 hour(s)
Proportion of subjects with a weekly RFBM rate ≥3 and an increase of at least 1 in the weekly RFBM rate from baseline
Proportion of subjects with an increase of at least 1 in the weekly RFBM rate from baseline
Weekly BM (bowel movement) rate
Weekly number of quality RFBMs (i.e. Bristol Stool Form Scale: types 3 and 4 being the “ideal stools”)
Weekly number of complete RFBMs (CRFBMs), i.e., RFBMs with a sensation of complete evacuation
Average of Bristol Stool Form Scale of RFBMs
Average of Straining Scale of RFBMs
Proportion of subjects with improvement in Bristol Stool Form Scale of RFBMs by ≥1 level
Proportion of subjects with improvement in Straining Scale of RFBMs by ≥1 level
Average percentage of RFBMs with Bristol Stool Form Scale type 3 or 4
Average percentage of RFBMs classified as diarrhea or watery stools
Proportion of subjects with any diarrhea or watery RFBMs (Bristol Stool Form Scale type 6 or 7)
Average percentage of RFBMs with Straining Scale scores of 0 or 1 (no, or mild)
Average percentage of RFBMs with a sensation of complete evacuation
Time to first RFBM from the first dose administration
Time to first BM from the first dose administration,
Response by prior MNTX use
PAC-SYM
PAC-QoL
EQ-5D
WPAI:GH
GCIC

### Safety Assessments

Vital signs
Rescue medication use
Concomitant medications
Adverse events, including serious adverse events
ECGs
Physical examinations
Laboratory evaluations

### Patient Reported Outcomes (PROs)

PROs were measured by the PAC-SYM, the PAC-QoL, EQ-5D, the WPAI:GH, and the GCIC. These assessments quantified the subjects' constipation symptoms, constipation-related quality of life, overall quality of life, change in bowel status, and degree of interference with ability to work. The total scale scores and associated subscales were calculated as well as their respective changes from baseline.

### Results

### Subjects

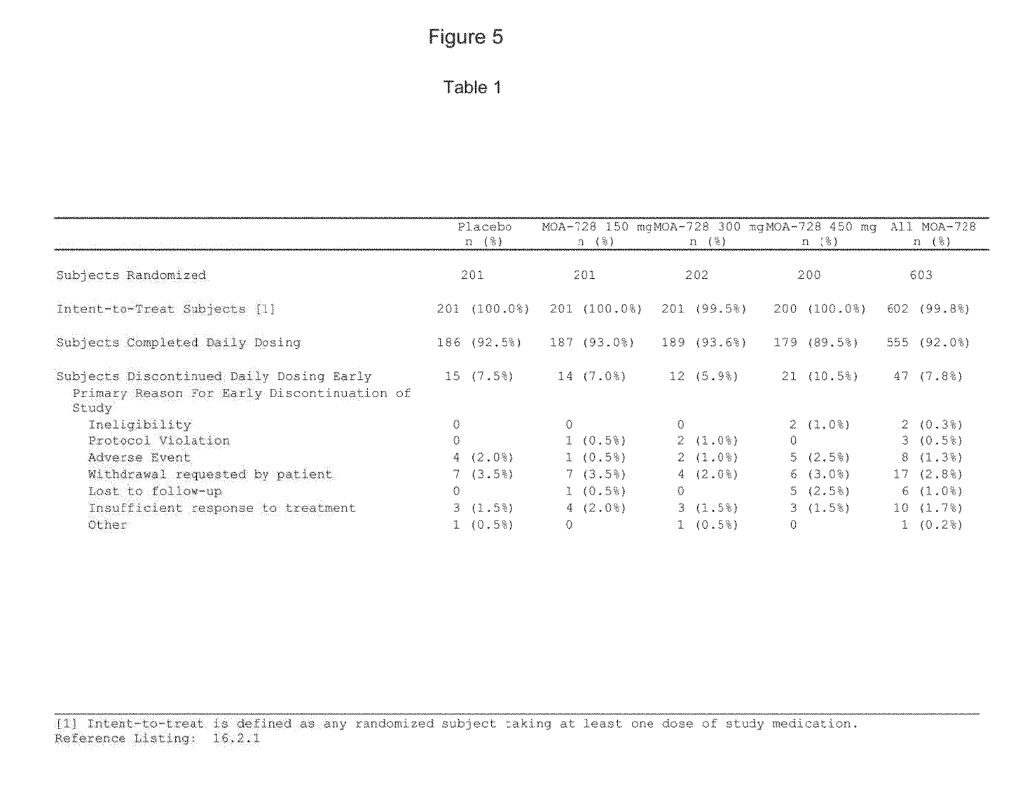

803 subjects enrolled in the study. As set forth in FIG. 5 (Table 1), of the 201 subject receiving placebo, 186 subjects completed the study. Of the 201 subjects receiving 150 mg oral methylnaltrexone daily, 187 subjects completed the study. Of the 201 subjects receiving 300 mg oral methylnaltrexone daily, 189 subjects completed the study. Finally, of the 200 subjects receiving 450 mg oral methylnaltrexone daily, 179 subjects completed the study.

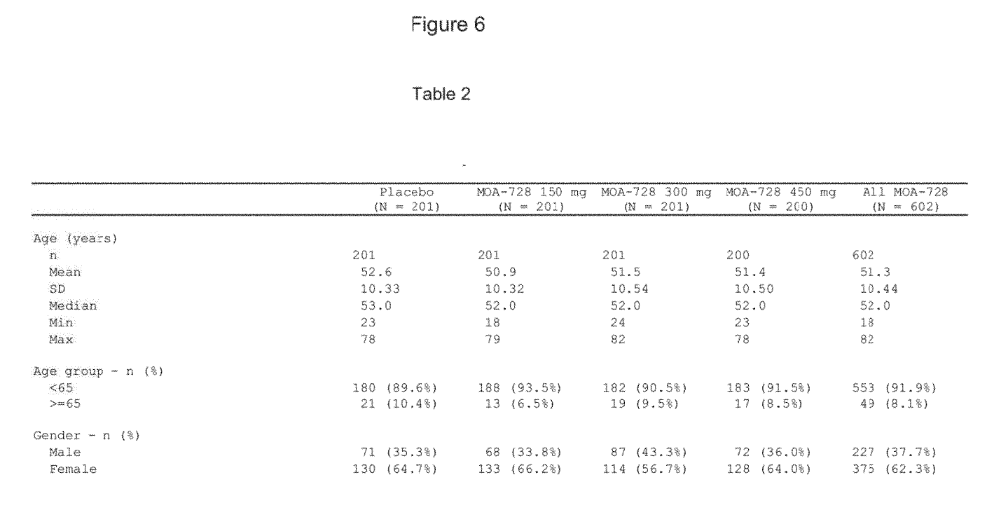

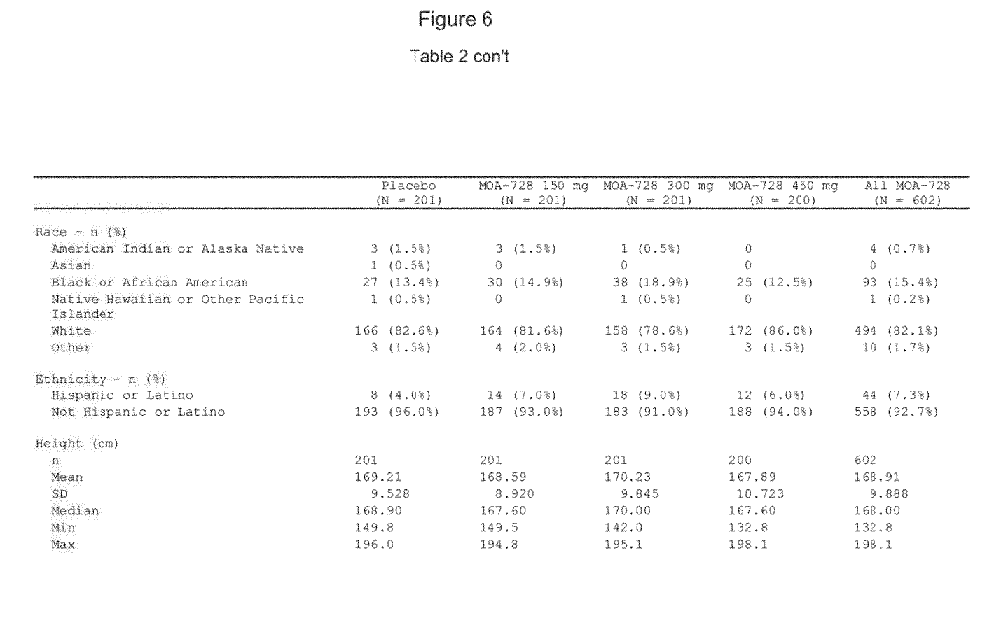

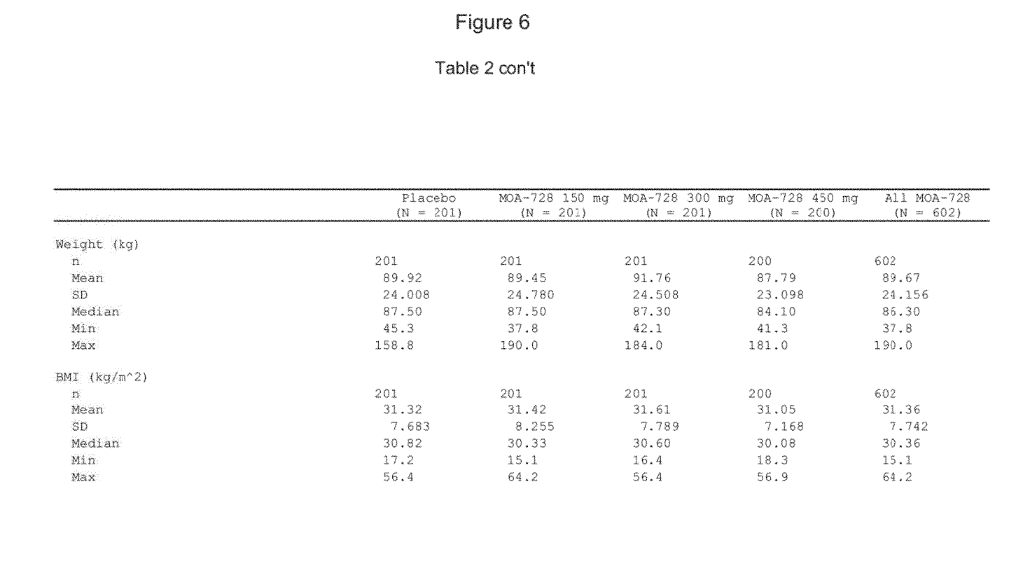

FIG. 6 (Table 2) provides the demographics for all the subjects enrolled in the study, including age, gender, race, ethnicity, height, weight and body mass index.

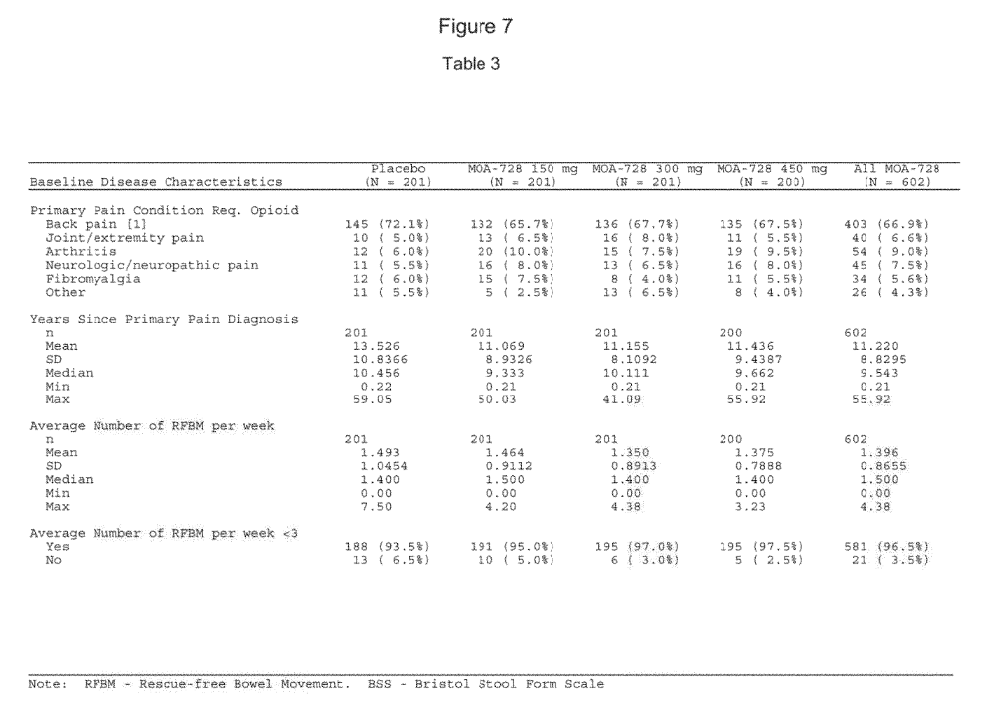

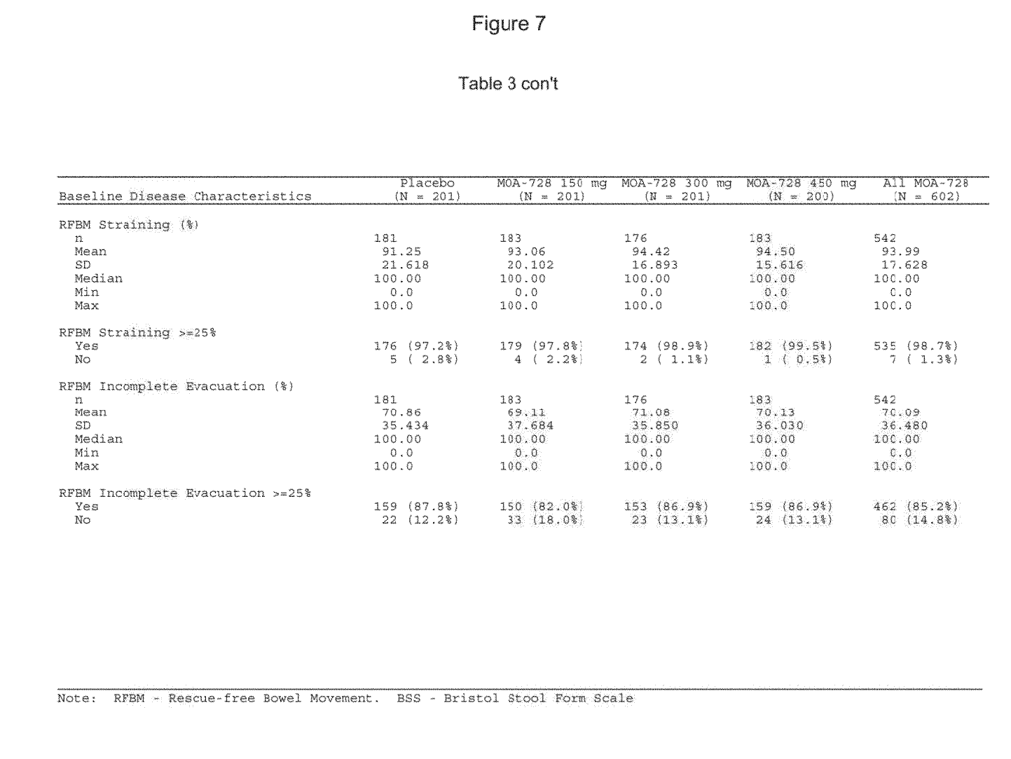

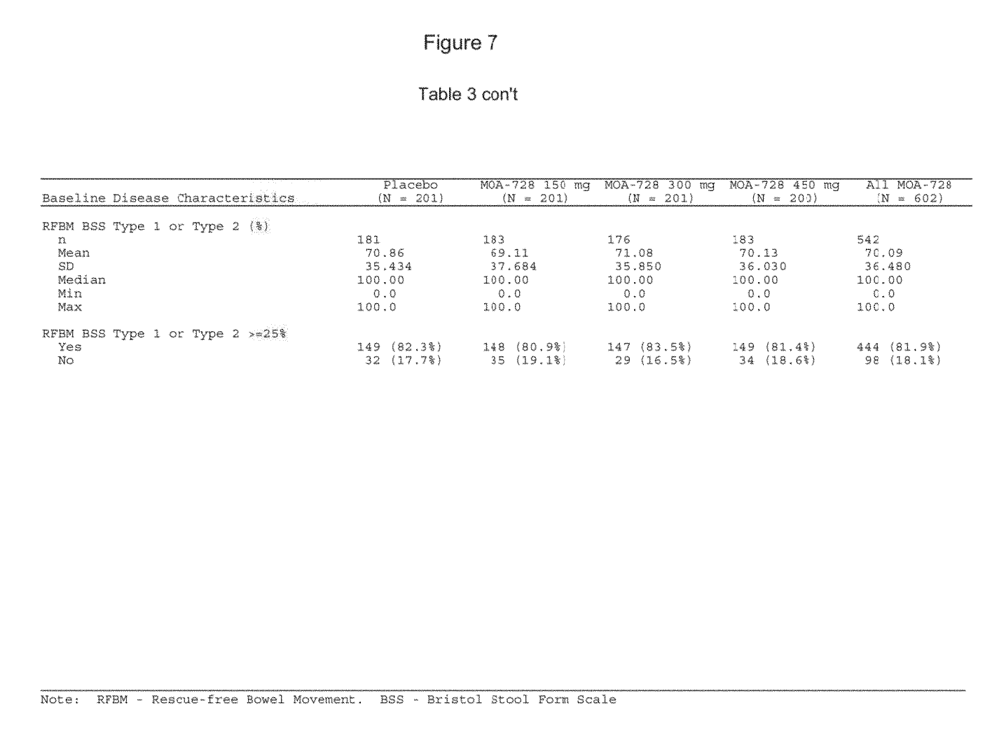

FIG. 7 (Table 3) provides the baseline disease characteristics for all subjects enrolled in the study. Specifically, FIG. 7 provides the nature of the non-malignant chronic pain experienced by the subject, including, for example, back pain, joint/extremity pain, arthritis, neurologic/neuropathic pain or fibromyalgia. FIG. 7 further provides (i) the average number of rescue free bowel movements per week for each subject, (ii) the average number of subjects having less than 3 rescue free bowel movements per week, (iii) the percentage of subjects experiencing straining during rescue free bowel movements; (iv) the percentage of subjects experiencing straining during at least 25% of rescue free bowel movements; (v) the percentage of subjects experiencing a sensation of incomplete evacuation following rescue free bowel movements; (vi) the percentage of subjects experiencing a sensation of incomplete evacuation following at least 25% of rescue free bowel movements; (vii) the percentage of subjects experiencing Bristol Stool Form Scale type 1 or 2 during rescue free bowel movements; and (vii) the percentage of subjects experiencing Bristol Stool Form Scale type 1 or 2 during at least 25% of rescue free bowel movements.

### Primary Efficacy Endpoints

Results demonstrate efficacy of the oral compositions of methylnaltrexone for each of the tested dosages, i.e., 150 mg, 300 mg and 450 mg of methylnaltrexone. Such efficacy is evidenced by demonstration of the primary efficacy endpoint, i.e., the average proportion of rescue free bowel movements per subject within 4 hours of all doses during the first 4 weeks of dosing.

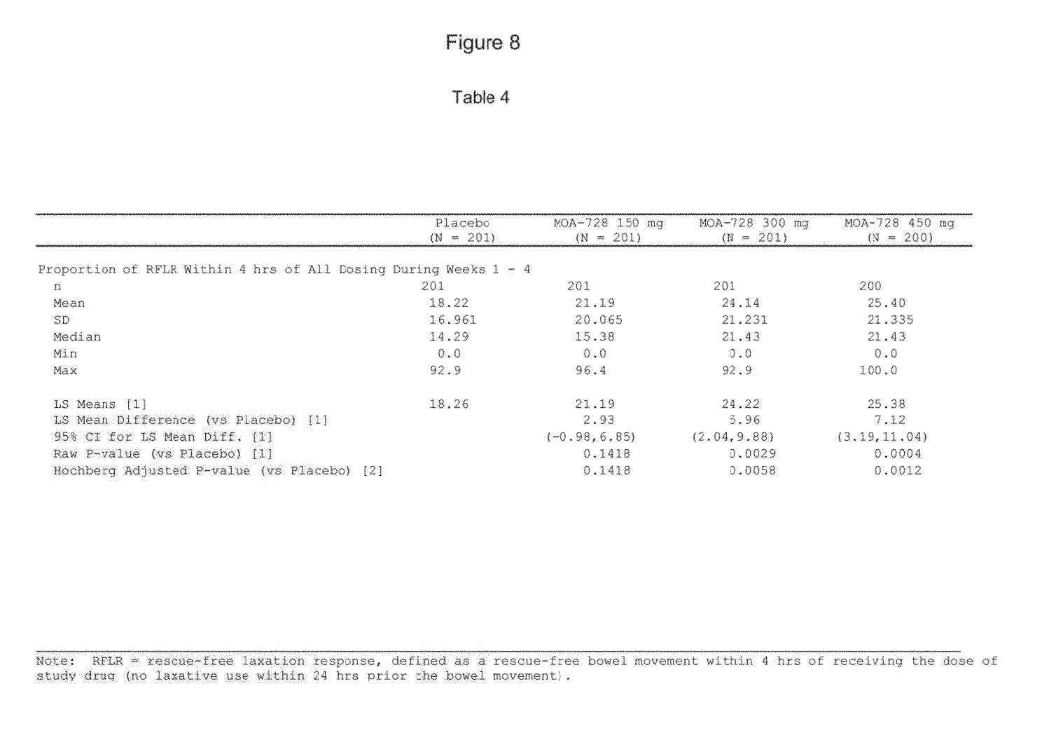

FIG. 8 (Table 4) summarizes the results with respect to the primary efficacy endpoint, i.e., the average proportion of rescue free bowel movements per subject within 4 hours of all doses during the first 4 weeks of the study as set forth in Example 1.

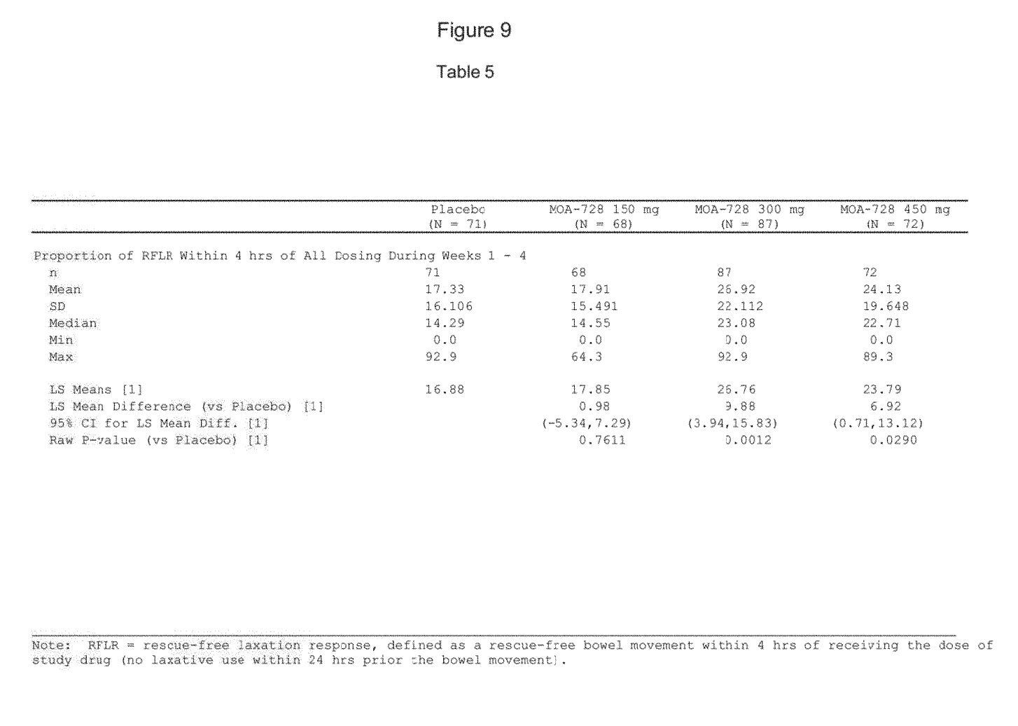

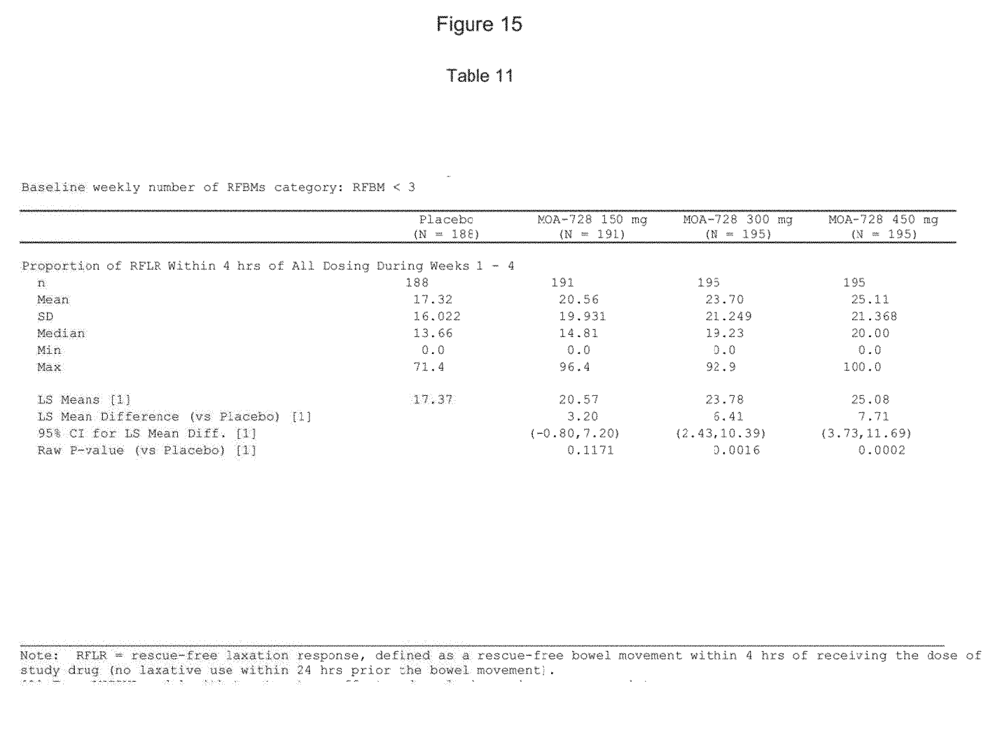

FIGS. 9-17 (Tables 5-13) further summarize the results with respect to the primary efficacy endpoint, wherein the results are categorized by the demographics of the subject or severity of the opioid induced constipation.

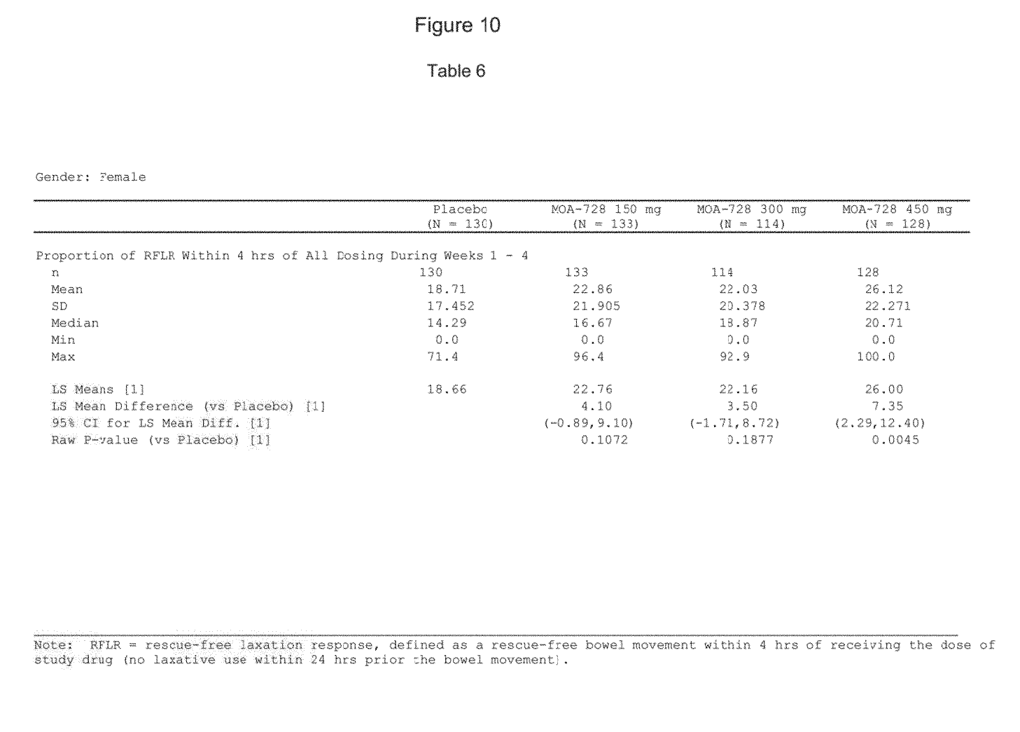

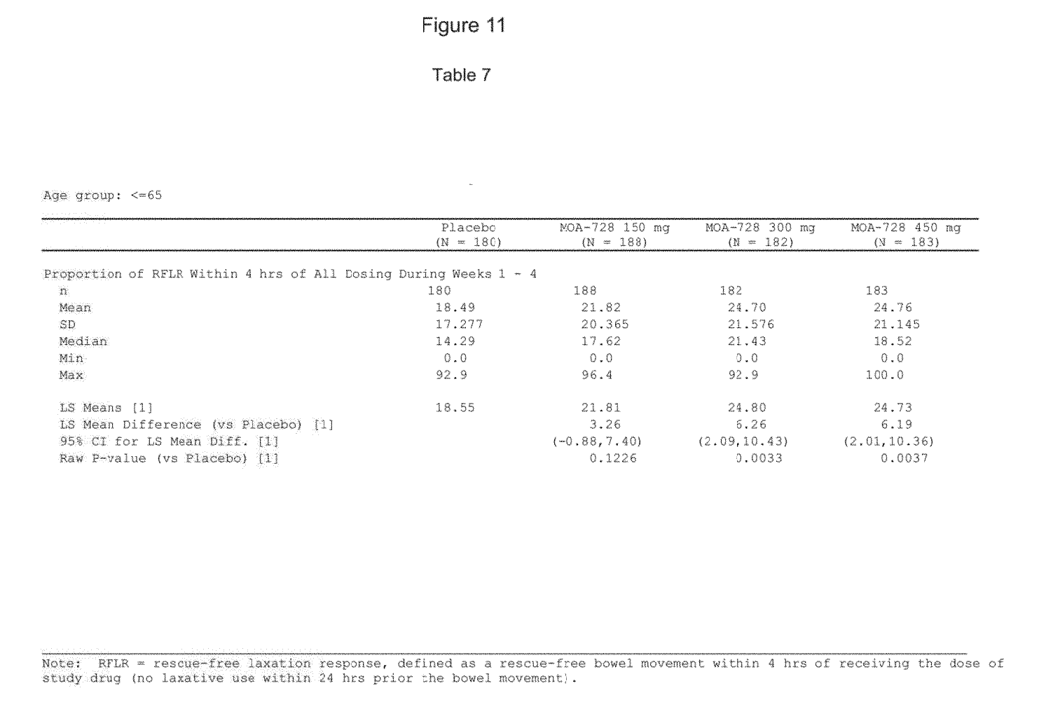

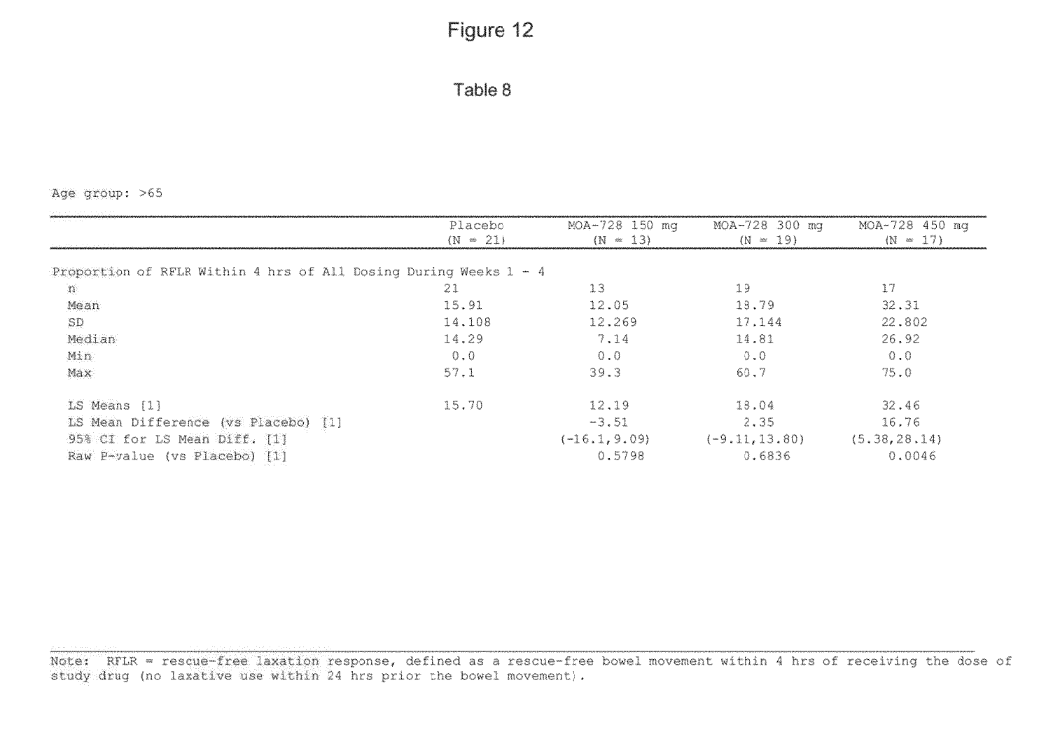

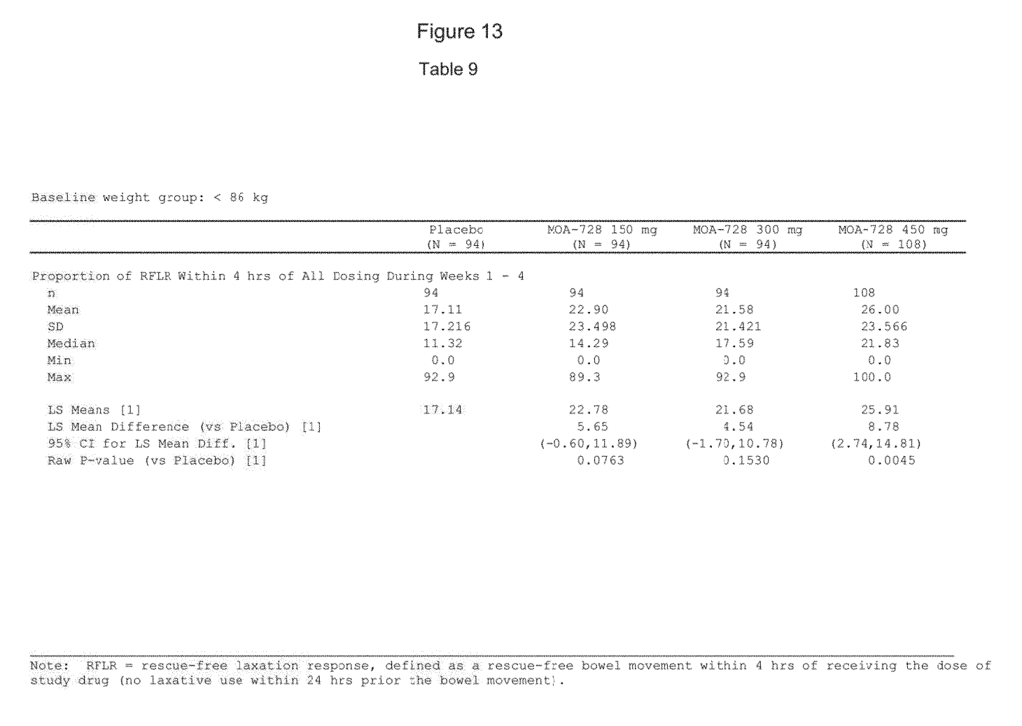

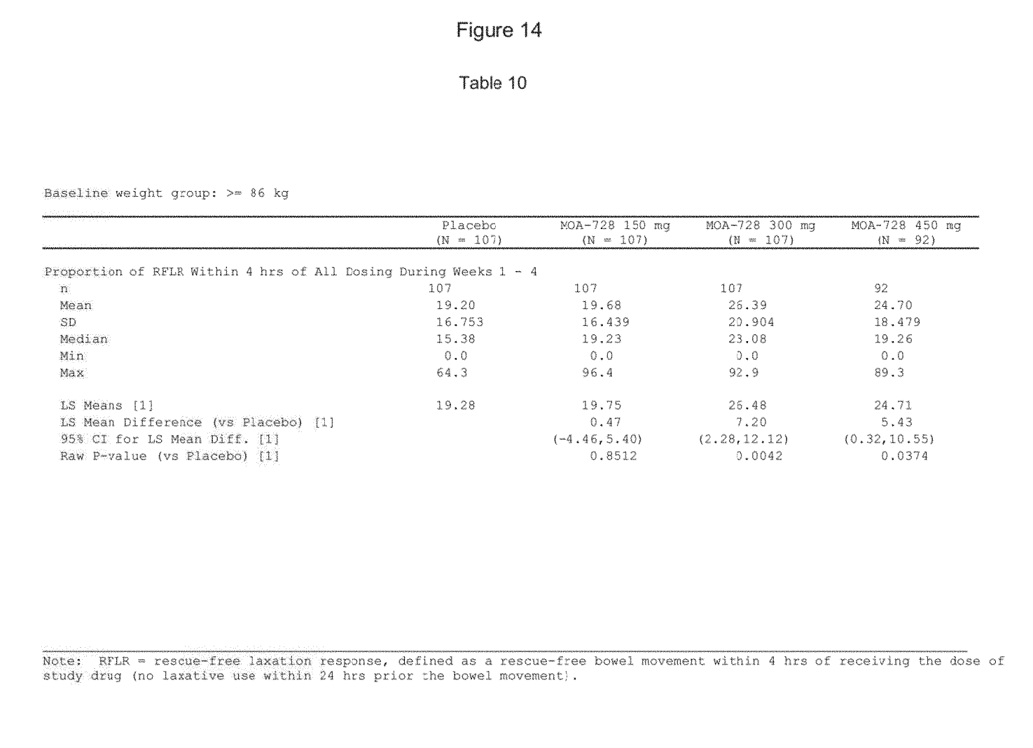

Specifically, FIGS. 9 and 10 (Tables 5 and 6) provide the results for male and female subjects, respectively, evidencing efficacy for both men and women. FIG. 11 (Table 7) demonstrates efficacy for subjects 65 years of age or younger, while FIG. 12 (Table 8) demonstrates for subjects older than 65. FIGS. 13 and 14 (Tables 9 and 10) provide results for subjects less than 86 kg and for subjects greater than or equal to 86 kg, respectively, each class of which exhibited efficacy with respect to the primary efficacy endpoint. Studies further demonstrate efficacy amongst white subjects, as evidenced by the primary efficacy endpoint.

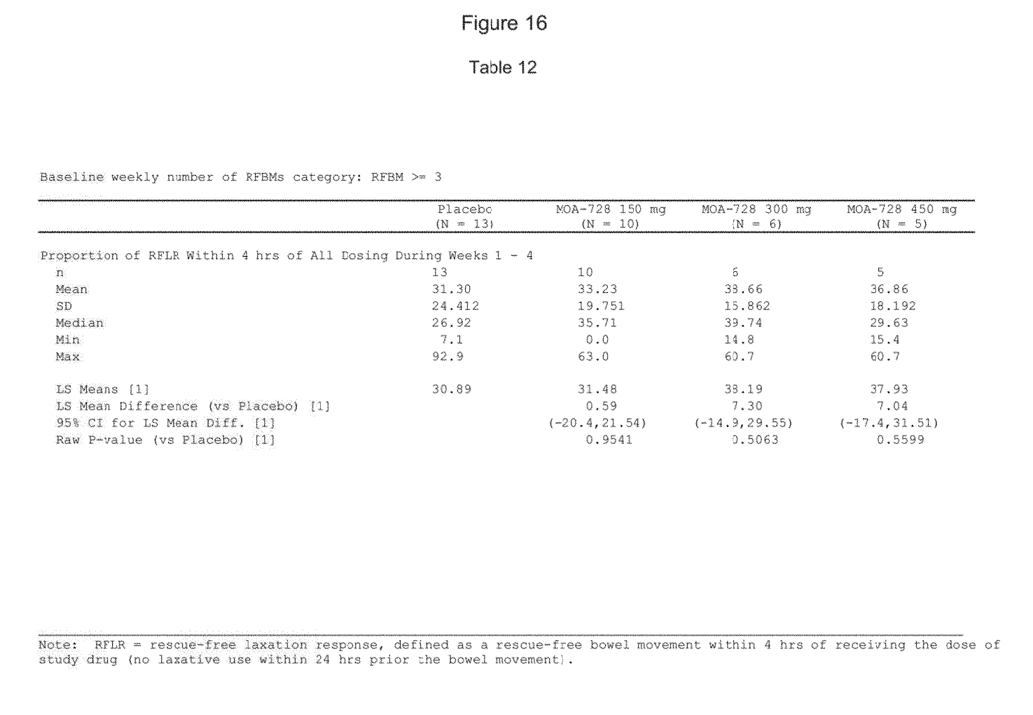

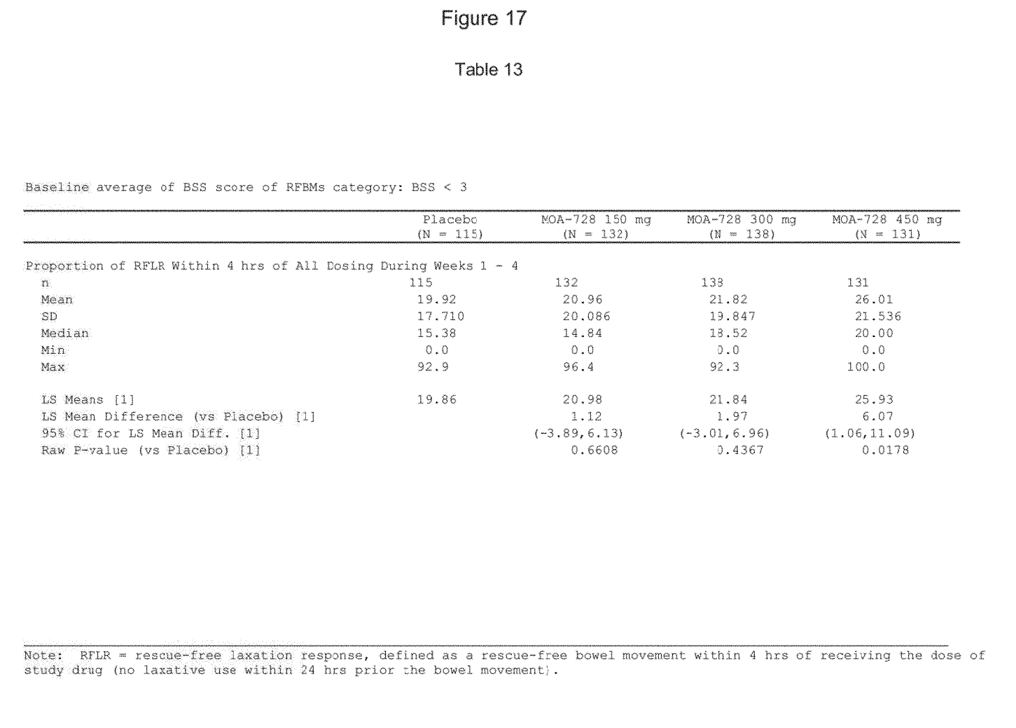

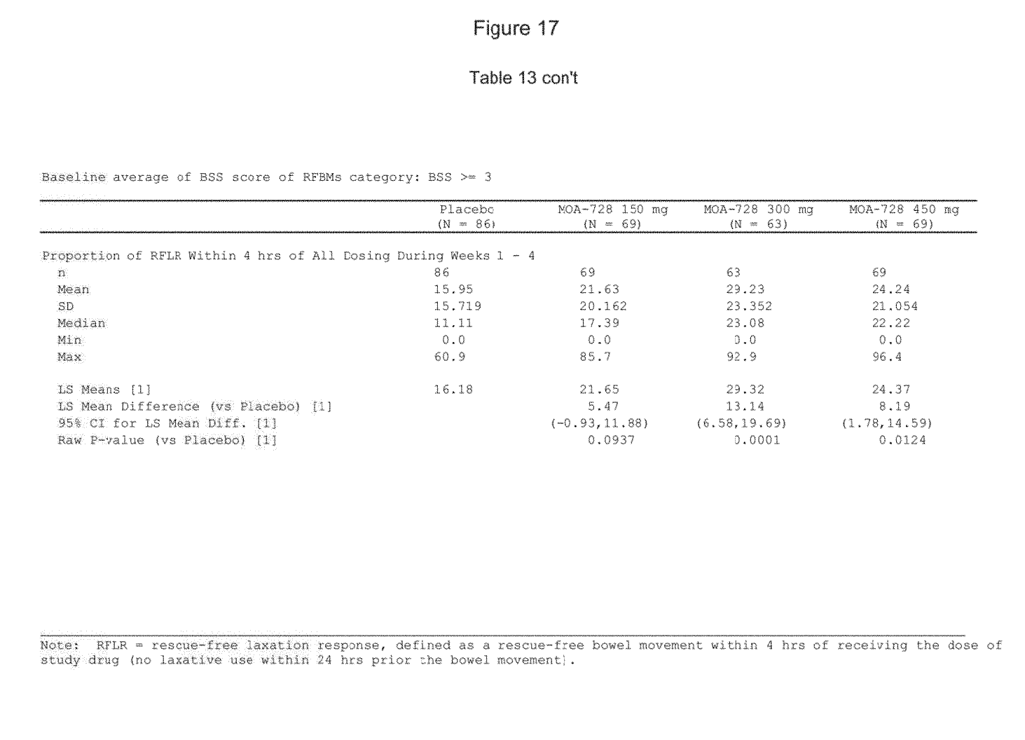

FIG. 16 (Table 11) confirms the primary efficacy for subjects having less than 3 rescue free bowel movements per week. Finally, FIG. 17 (Table 13) confirms the primary efficacy for subjects having a Bristol Stool Form Scale Score less than 3.

### Secondary Efficacy Endpoints

Results further demonstrate efficacy of the oral compositions of methylnaltrexone for each of the tested dosages, i.e., 150 mg, 300 mg and 450 mg of methylnaltrexone, as evidenced by confirmation of the secondary efficacy endpoints including:

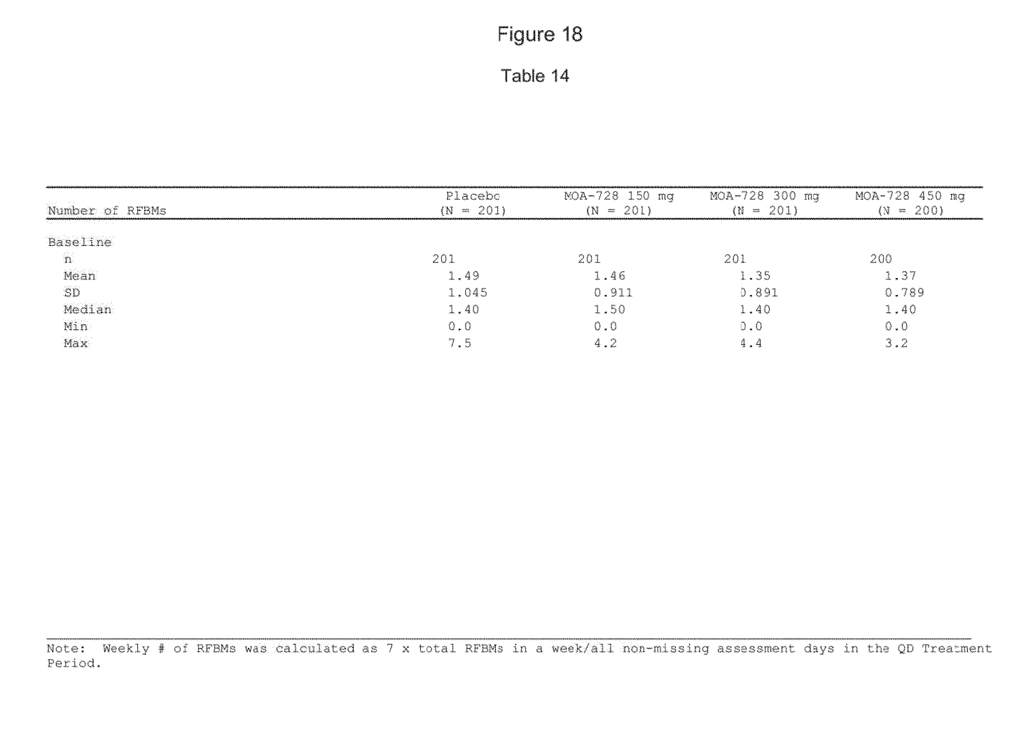

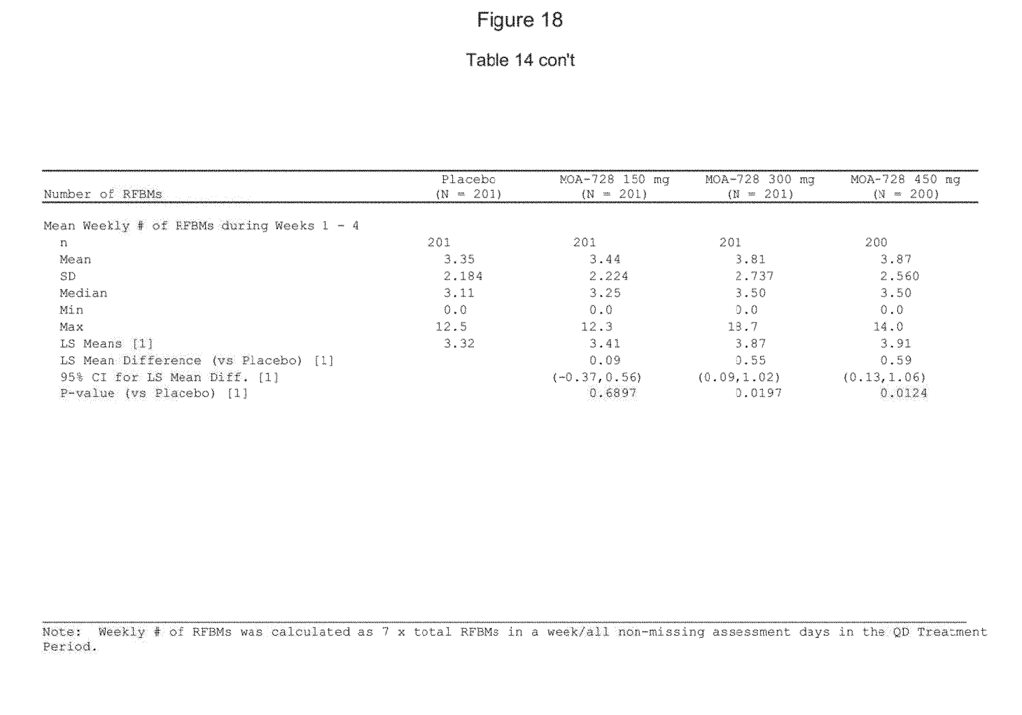

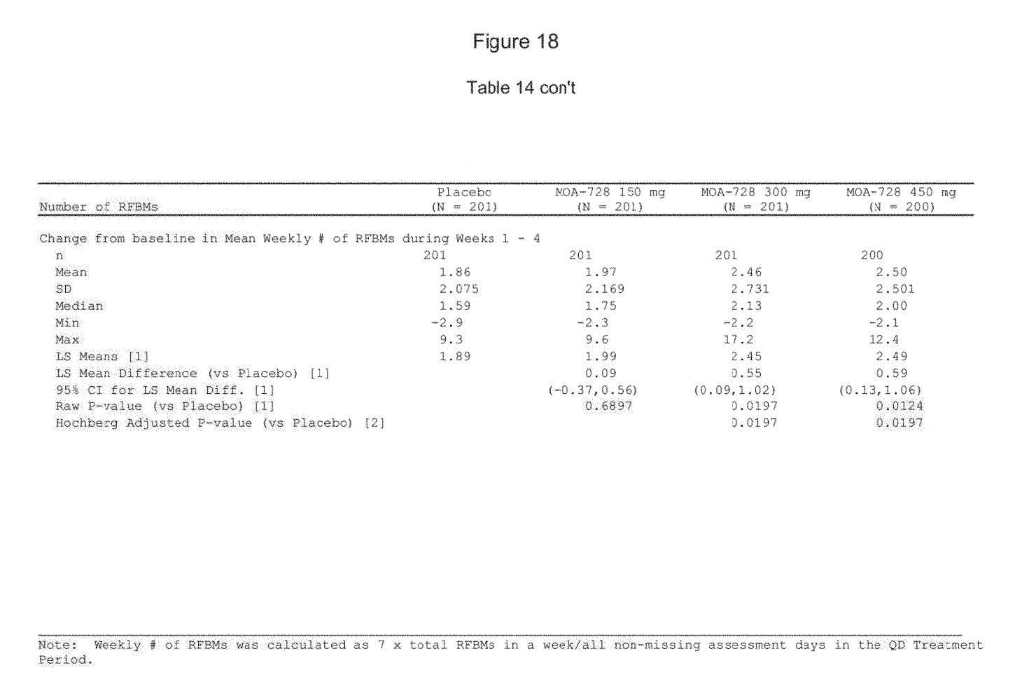

(a) change in weekly number of rescue free bowel movements from baseline during weeks 1-4 of the study (see FIG. 18; Table 14); and

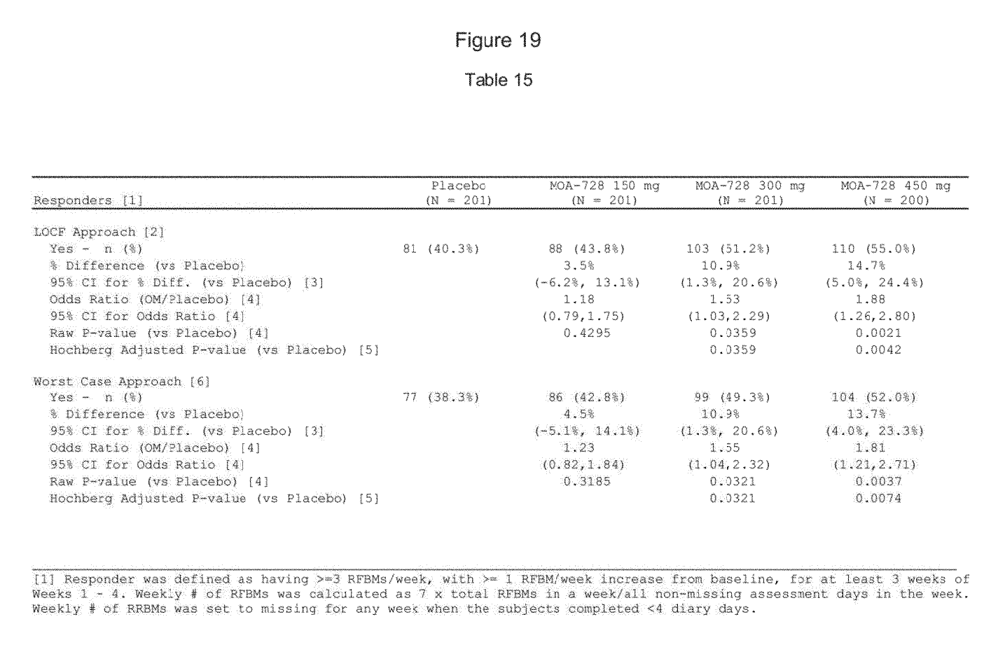

(b) response to study drug, defined as having at least 3 rescue free bowel movements per week for each of the first 4 weeks of the study with an increase of at least one rescue free bowel movement over baseline for at least 3 weeks of the first 4 weeks of the study (see FIG. 19; Table 15).

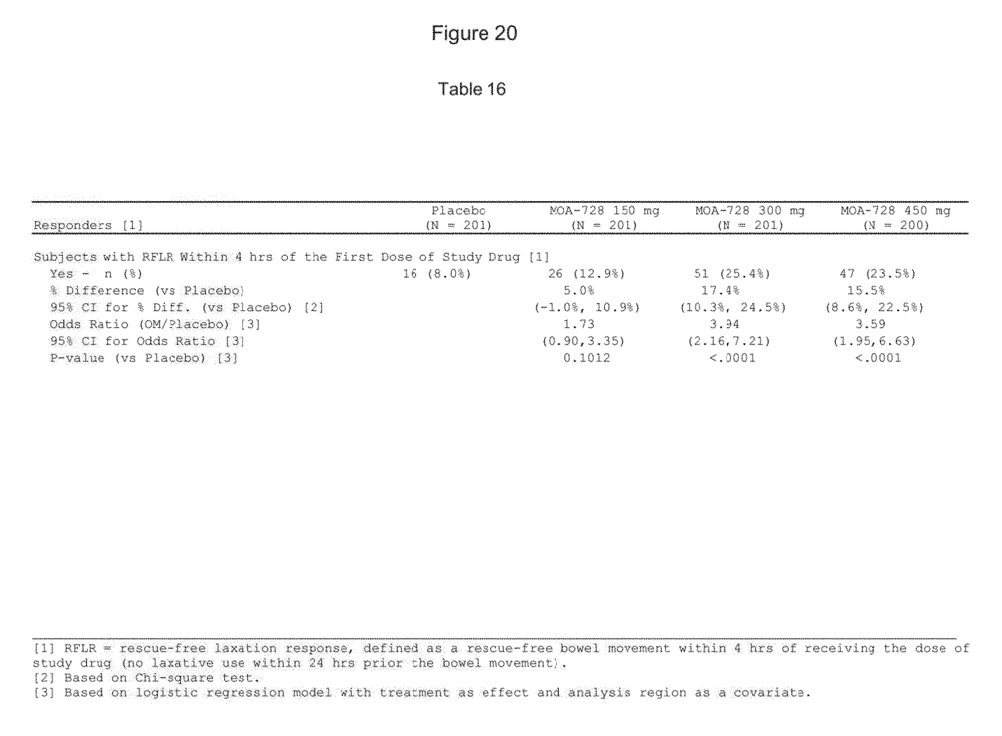

Moreover, another secondary endpoint further confirmed efficacy of the study drug as depicted in FIG. 20 (Table 16) which sets forth the proportion of subjects with rescue free bowel movements within 4 hours of the first dose of study drug.

### Adverse Events

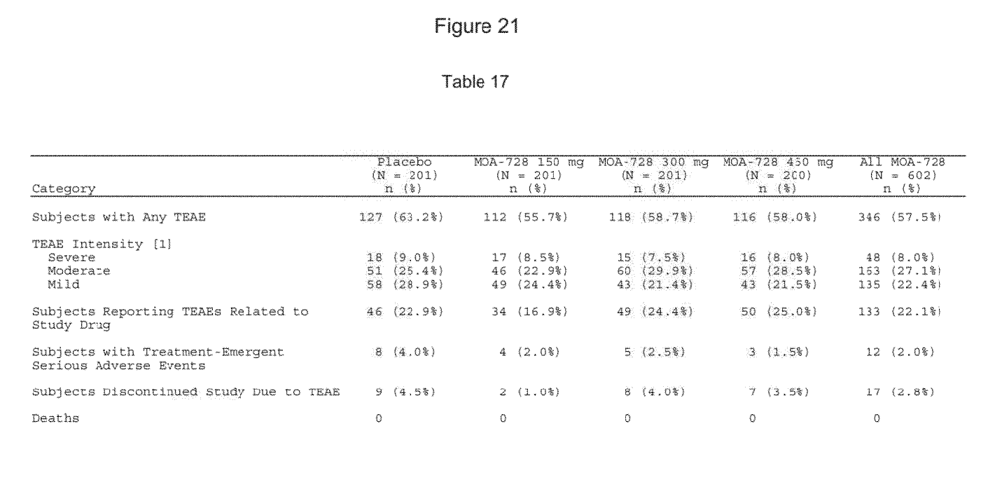

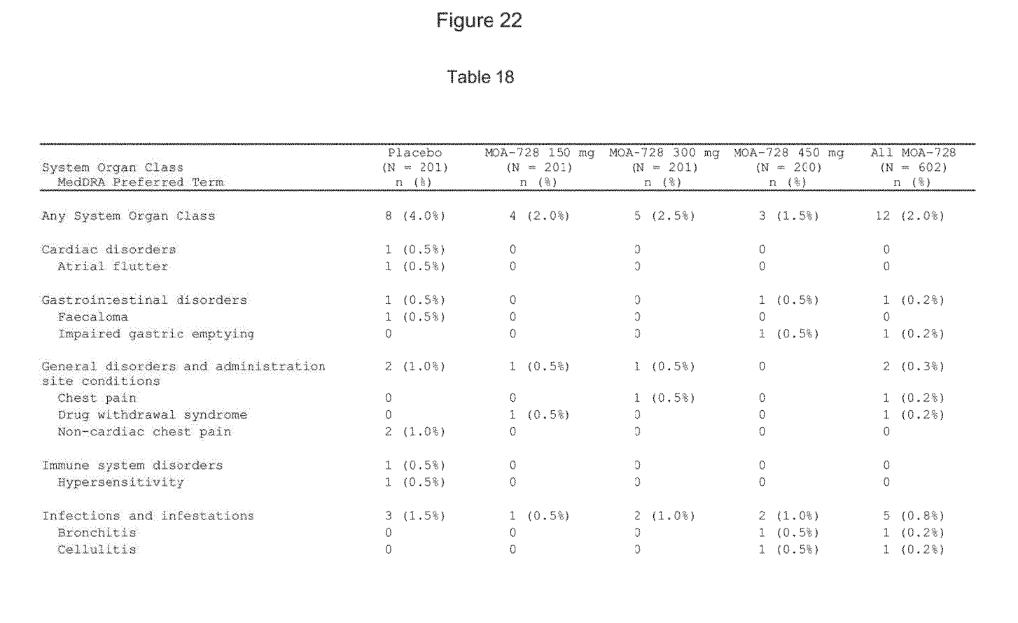

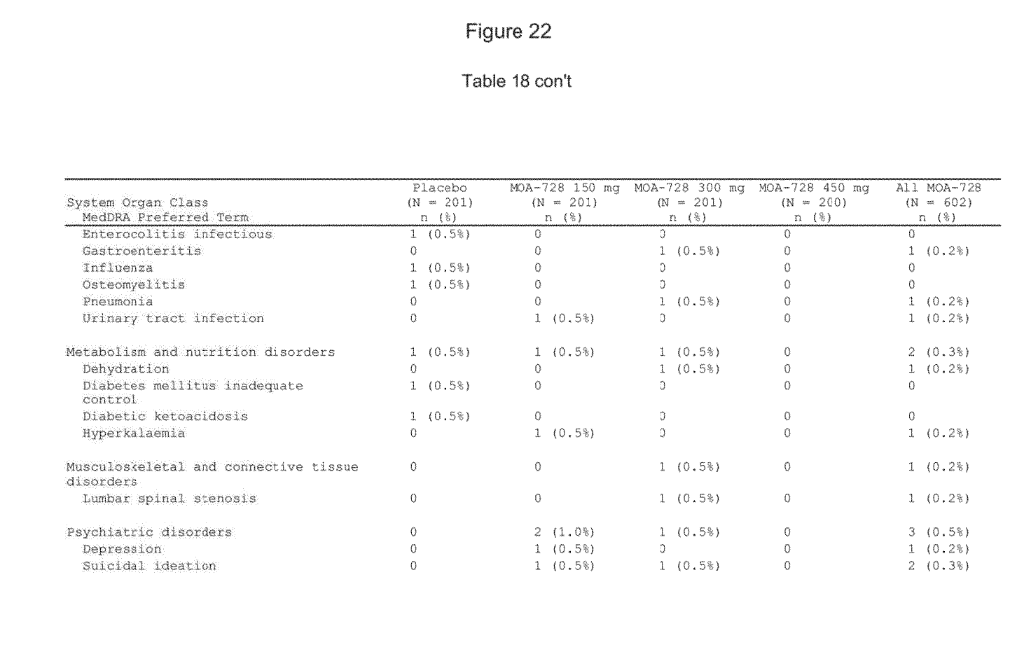

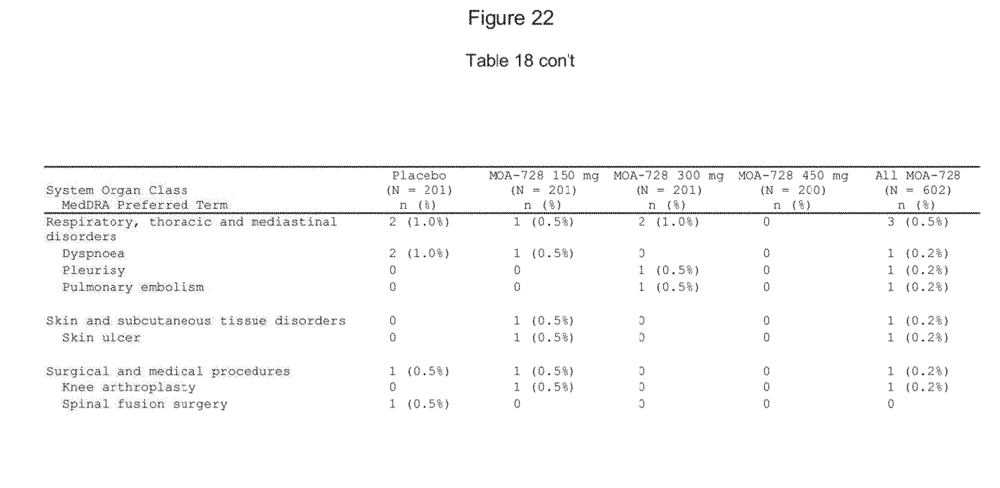

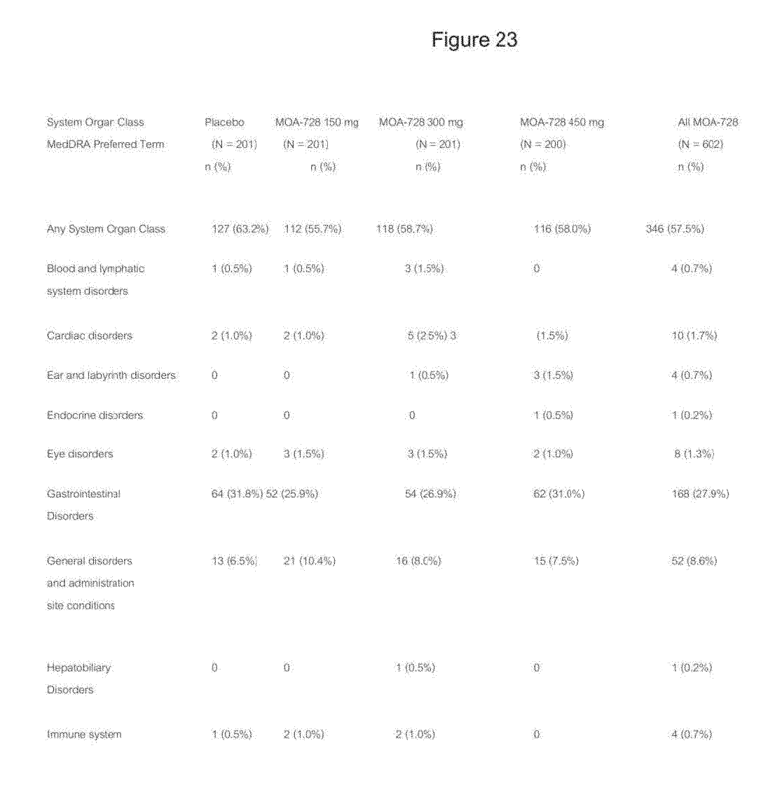

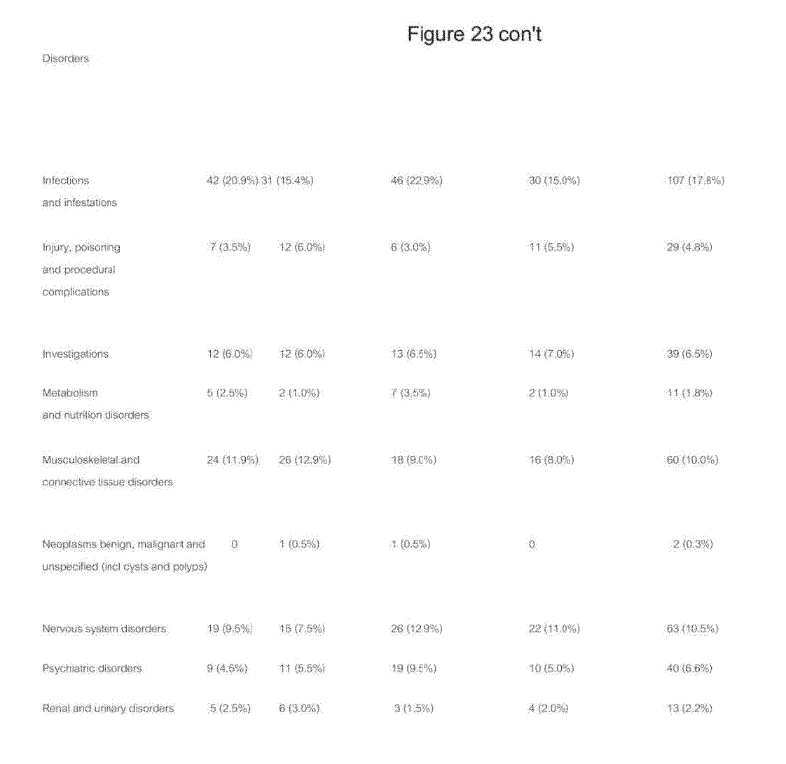

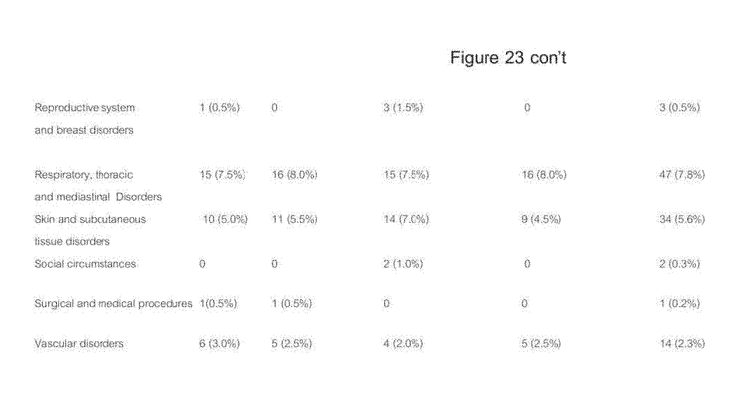

Results further demonstrate that study drug, at dosages of 150 mg, 300 mg and 450 mg, did not result in adverse events as set forth in each of FIG. 21 (all adverse events), FIG. 22 (serious adverse events organized by organ system class) and FIG. 23 (adverse events organized by organ system class).

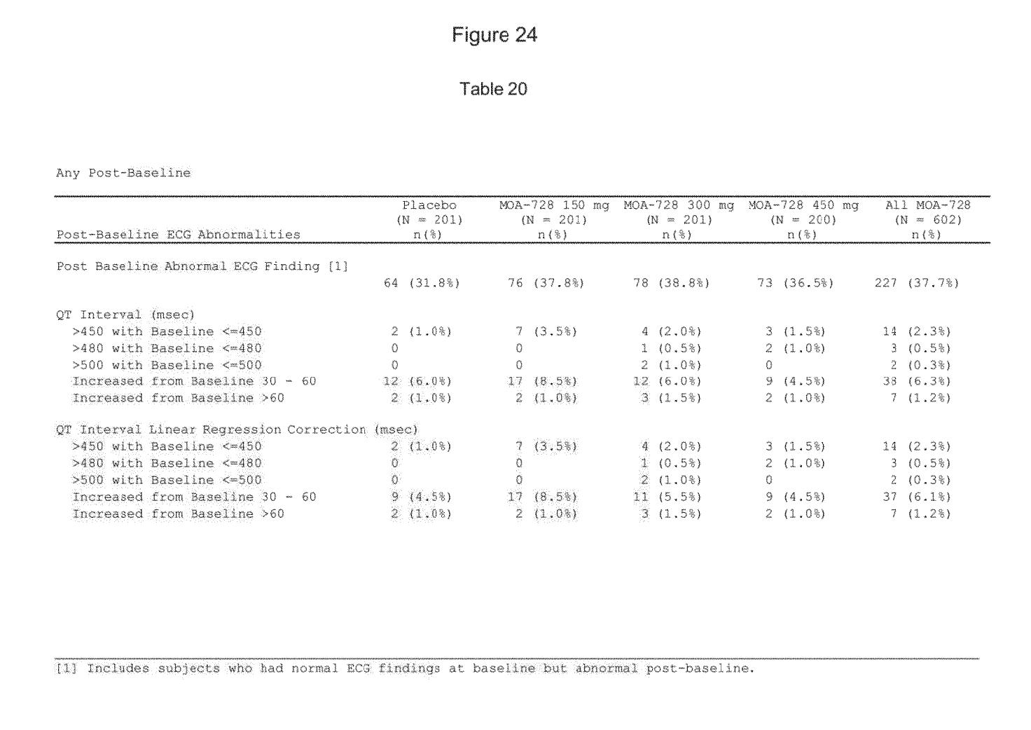

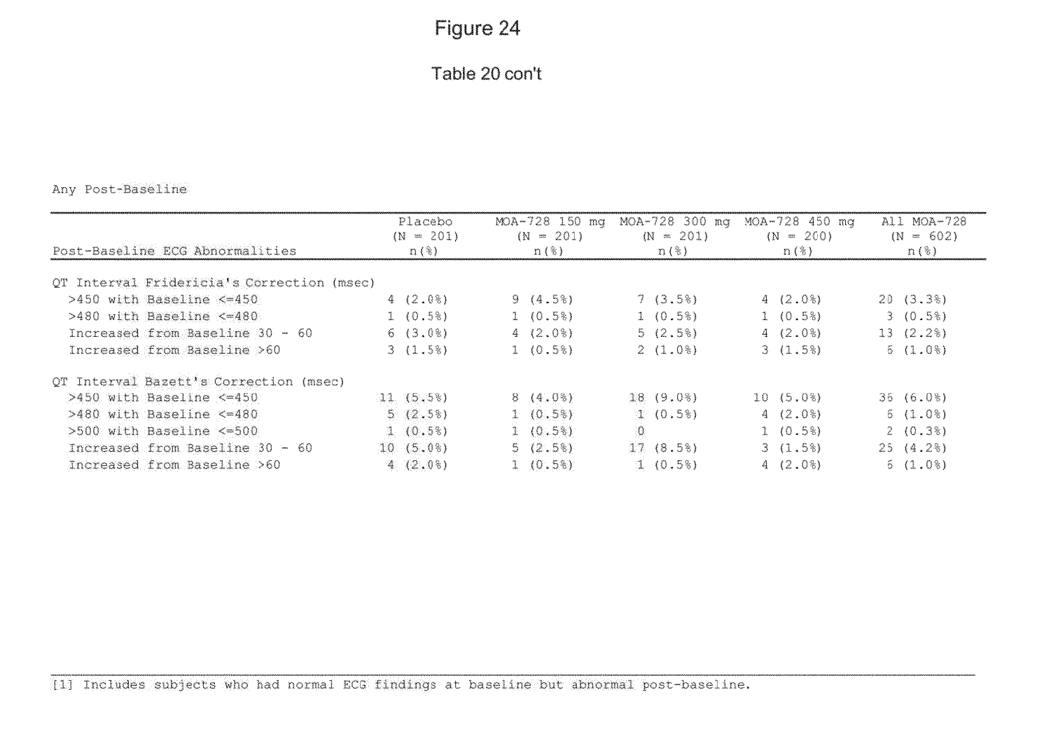

Finally, FIG. 24 (Table 20) summarizes clinically significant electrocardiogram results as set forth in Example 1.

### Example 2: Preparation of Tablets of Methylnaltrexone Bromide

Methylnaltrexone bromide may be prepared according to the methods described in detail in international PCT Patent Application publication number, WO 2006/127899. Formulations containing methylnaltrexone were prepared using pharmaceutically acceptable excipients. Spheroids containing methylnaltrexone were prepared. Tablets were prepared from spheroids, using conventional techniques. The tablets dissolve in under 10 minutes.

The spheroids were prepared by a wet granulation process followed by extrusion and spheronization, as described in the following general method. Methylnaltrexone bromide and pharmaceutically acceptable excipients were combined in an aqueous solution. Water was added until wet mass suitable for extrusion was obtained. The wet mass was passed through an extruder, and the extrudate was spheronized in a spheronizer. The resulting spheroids were dried in a fluid bed drier and passed through a screen. The uncoated spheroids were stored in appropriate container.

### Example 3: Clinical Pharmacokinetics of Orally Administered Methylnaltrexone

Presented herein is a clinical pharmacokinetics study, Study C, as well as Studies A and B. Study A investigated the single and multiple dose pharmacokinetics of methylnaltrexone (MNTX) and its metabolites (M2: methylnaltrexone sulfate; M4: 6α-methylnaltrexol; and M5: 6β-methylnaltrexol) following the subcutaneous administration of 12 mg methylnaltrexone. In Study B, the single and multiple dose pharmacokinetics of methylnaltrexone (MNTX) and its metabolites (M2, M4, and M5) were examined following a 20-minute short intravenous infusion of 24 mg of methylnaltrexone (MNTX).

In Study C, the pharmacokinetics of methylnaltrexone (MNTX) and its 3 metabolites (M2, M4 and M5) were investigated in two stages: 1) single and multiple dose pharmacokinetics of MNTX and 3 metabolites, (M2, M4 and M5) following MNTX 450 mg PO×7 days, and 2) the relative MNTX bioavailability following single oral dose administration of 450 mg MNTX as uncoated and film-coated 150-mg MNTX tablets. In addition, the urinary elimination of MNTX was characterized.

Pharmacokinetic parameters included Cₘₐₓ, AUCₜ, AUC_{inf}, tₘₐₓ, t_{1/2}, % Re₂₄, accumulation factor (R) as defined below and metabolite/parent drug ratio.

R=Accumulation Factor(based on AUC₀₋₂₄(ng·h/mL):Day 7 AUC AUC₀₋₂₄/Day 1 AUC₀₋₂₄

Metabolite-Parent Drug ratio(based on ng·h/mL)(%)=100*(Metabolite AUC₂₄/MNTX AUC₂₄)

Note: AUCinf was used in place of AUC AUC₀₋₂₄ for R and Metabolite-Parent Drug ratio computations following IV administration. Results are summarized in Tables 21 and 22.

TABLE 21
Single and Multiple Dose Pharmacokinetics [Mean (SD)] of Methylnaltrexone
(MNTX) and its metabolites of Study C; Compared to Studies A and B as Noted

| | | Analyte | | | | | | | |
|---|---|---|---|---|---|---|---|---|---|
| | | MNTX | | | | M2 | | | |
| Dosage form | PK Parameter | Day 1 | | Day 7 | | Day 1 | | Day 7 | |
| 450 mg | AUC_{inf} | 314.53 | (134.72) | 403.72 | (142.92) | 216.89 | (100.64) | 320.51 | (166.55) |
| Tablets | (ng · h./mL) | | | | | | | | |
| 12 mg SC | AUC_{inf} | 223.00 | (29.1) | 223.0 | (28.2) | 71.9 | (23.3) | 66.3 | (16.7) |
| Injection | (ng · h./mL) | | | | | | | | |
| 24 mg IV | AUC_{inf} | 396 | (74) | 375 | (74) | 162 | (79) | 176 | (72) |
| infusion¹ | (ng · h./mL) | | | | | | | | |
| 450 mg | AUC₀₋₂₄ | 280.16 | (125.35) | 308.89 | (102.34) | 188.63 | (85.48) | 243.72 | (137.50) |
| Tablets | (ng · h./mL) | | | | | | | | |
| 12 mg SC | AUC₀₋₂₄ | 217.95 | (28.28) | 223.18 | (28.2) | 61.34 | (21.32) | 66.3 | (16.69) |
| Injection | (ng · h./mL) | | | | | | | | |

| | | | | | |
|---|---|---|---|---|---|
| 24 mg IV | AUC₀₋₆ | 326 | (66) | 72.3 | (34.7) |
| infusion¹ | (ng · h./mL) | | | | |

| | | | | | |
|---|---|---|---|---|---|
| 450 mg | Metabolite/MNTX | 72.69 | (28.59) | 79.11 | (39.28) |
| Tablets | Ratio (%) | | | | |
| 12 mg SC | Metabolite/MNTX | 28.71 | (8.30) | 29.30 | (6.32) |
| Injection | Ratio (%) | | | | |

| | | | |
|---|---|---|---|
| 24 mg IV | Metabolite ÷ | 46.60 | (15.6) |
| infusion¹ | MNTX Ratio (%) | | |

| | | | | | |
|---|---|---|---|---|---|
| 450 mg | R (PO) | 1.20 | (0.32) | 1.30 | (0.38) |
| Tablets | | | | | |
| 12 mg SC | R (SC) | 1.05 | (0.064) | 1.13 | (0.10) |
| Injection² | | | | | |
| 24 mg IV | R (IV) | 1.17 | (0.2) | 2.61 | (0.73) |
| infusion¹ | | | | | |

| | | | | |
|---|---|---|---|---|
| 450 mg | % Re₂₄ (% Dose) | 3.25 | (1.29) | N/A |
| Tablets | | | | |

| | | Analyte | | | | | | | |
|---|---|---|---|---|---|---|---|---|---|
| | | M4 | | | | M5 | | | |
| Dosage form | PK Parameter | Day 1 | | Day 7 | | Day 1 | | Day 7 | |
| 450 mg | AUC_{inf} | 124.23 | (50.83) | 221.13 | (108.73) | 73.61 | (33.77) | 120.87 | (56.62) |
| Tablets | (ng · h./mL) | | | | | | | | |
| 12 mg SC | AUC_{inf} | 38.3 | (10.6) | 41.9 | (13.5) | 18.5 | (6.55) | 19.5 | (6.26) |
| Injection | (ng · h./mL) | | | | | | | | |
| 24 mg IV | AUC_{inf} | 61.30 | (25.4) | 54.0 | (15.9) | 35.10 | (11.7) | 30.0 | (8.7) |
| infusion¹ | (ng · h./mL) | | | | | | | | |
| 450 mg | AUC₀₋₂₄ | 79.73 | (39.06) | 119.61 | (57.43) | 40.84 | (19.31) | 66.33 | (31.05) |
| Tablets | (ng · h./mL) | | | | | | | | |
| 12 mg SC | AUC₀₋₂₄ | 34.66 | (11.12) | 41.86 | (13.47) | 14.41 | (4.54) | 19.51 | (6.26) |
| Injection | (ng · h./mL) | | | | | | | | |
| 24 mg IV | AUC₀₋₆ | 28.8 | (12.0) | | | 12.3 | (5.00) | | |
| infusion¹ | (ng · h./mL) | | | | | | | | |
| 450 mg | Metabolite/MNTX | 29.70 | (10.34) | 38.50 | (12.23) | 15.10 | (5.46) | 21.41 | (6.91) |
| Tablets | Ratio (%) | | | | | | | | |
| 12 mg SC | Metabolite/MNTX | 15.81 | (4.45) | 18.75 | (6.05) | 6.58 | (1.79) | 8.72 | (2.69) |
| Injection | Ratio (%) | | | | | | | | |

| | | | | | |
|---|---|---|---|---|---|
| 24 mg IV | Metabolite ÷ | 14.90 | (3.8) | 8.69 | (1.96) |
| infusion¹ | MNTX Ratio (%) | | | | |

| | | | | | |
|---|---|---|---|---|---|
| 450 mg | R (PO) | 1.62 | (0.56) | 1.76 | (0.61) |
| Tablets | | | | | |
| 12 mg SC | R (SC) | 1.25 | (0.18) | 1.42 | (0.24) |
| Injection² | | | | | |
| 24 mg IV | R (IV) | 2.08 | (0.55) | 2.91 | (0.99) |
| infusion¹ | | | | | |

| | | | | | |
|---|---|---|---|---|---|
| 450 mg | % Re₂₄ (% Dose) | N/A | N/A | N/A | N/A |
| Tablets | | | | | |

¹data taken from Study B, a study of 24 mg given as a short infusion.
²data taken from Study A, a study of 12 mg given sc.
* Harmonic mean (harmonic SD) % Re = % dose excreted by renal route, R = AUC₀₋₂₄ on Day 7/AUC₀₋₂₄ on Day 1, % Re₂₄ = % oral dose excreted in urine in 24 hr

TABLE 22
Single and Multiple Dose Pharmacokinetic Parameters [Mean (SD)]
for Methylnaltrexone (MNTX) and its metabolites (cont.).

| | | Analyte | | | | | | | |
|---|---|---|---|---|---|---|---|---|---|
| | | MNTX | | | | M2 | | | |
| Dosage form | PK Parameter | Day 1 | | Day 7 | | Day 1 | | Day 7 | |
| 450 mg | Cₘₐₓ | 47.05 | (22.88) | 45.50 | (23.58) | 17.15 | (8.09) | 21.00 | (11.50) |
| Tablets | (ng/mL) | | | | | | | | |
| 12 mg SC | Cₘₐₓ | 139.89 | (35.6) | 119.1 | (27.19) | 6.34 | (2.66) | 5.70 | (1.32) |
| Injection | (ng/mL) | | | | | | | | |
| 24 mg IV | Cₘₐₓ | 533 | (103) | 520 | (103) | 16.6 | (7.8) | 37.70 | (15.1) |
| infusion | (ng/mL) | | | | | | | | |
| 450 mg | Tₘₐₓ | 2.00 | (0.50-4.03) | 2.00 | (0.50-4.03) | 4.02 | (4.00-4.84) | 4.02 | (4.00-8.00) |
| Tablets | (h) | | | | | | | | |
| 12 mg SC | Tₘₐₓ | 0.25 | (0.25-0.5) | 0.25 | (0.25-0.5) | 4.00 | (4.0-8.0) | 4.00 | (4.0-8.0) |
| Injection | (h) | | | | | | | | |
| 450 mg | t_{1/2}* | 8.805 | (2.24) | 19.22 | (4.98) | 7.19 | (1.62) | 13.87 | (6.92) |
| Tablets | (h) | | | | | | | | |

| | | | | | |
|---|---|---|---|---|---|
| 12 mg SC | t_{1/2}* | 5.33 | (NC) | 5.57 | (NC) |
| Injection | (h) | | | | |
| 24 mg IV | t_{1/2} | 10.8 | (1.70) | 5.70 | (1.4) |
| infusion | (h) | | | | |

| | | Analyte | | | | | | | |
|---|---|---|---|---|---|---|---|---|---|
| | | M4 | | | | M5 | | | |
| Dosage form | PK Parameter | Day 1 | | Day 7 | | Day 1 | | Day 7 | |
| 450 mg | Cₘₐₓ | 9.01 | (5.74) | 10.77 | (5.22) | 3.48 | (2.09) | 4.89 | (2.30) |
| Tablets | (ng/mL) | | | | | | | | |
| 12 mg SC | Cₘₐₓ | 4.64 | (2.14) | 4.33 | (1.55) | 1.17 | (0.554) | 1.42 | (0.444) |
| Injection | (ng/mL) | | | | | | | | |
| 24 mg IV | Cₘₐₓ | 11.0 | (5.5) | 18.10 | (6.0) | 3.44 | (1.61) | 8.71 | (2.4) |
| infusion | (ng/mL) | | | | | | | | |
| 450 mg | Tₘₐₓ | 2.0003 | (1.005-4.027) | 2.0007 | (1.20) | 3.13 | (1.03) | 2.69 | (1.08) |
| Tablets | (h) | | | | | | | | |
| 12 mg SC | Tₘₐₓ | 1.0 | (0.5-2.0) | 1.0 | (0.5-4.0) | 2.0 | (0.5-8.0) | 2.0 | (1.0-6.0) |
| Injection | (h) | | | | | | | | |
| 450 mg | t_{1/2}* | 17.48 | (7.21) | 31.85 | (5.05) | 18.40 | (6.94) | 28.65 | (5.52) |
| Tablets | (h) | | | | | | | | |

| | | | | | |
|---|---|---|---|---|---|
| 12 mg SC | t_{1/2}* | 7.59 | (NC) | 8.13 | (NC) |
| Injection | (h) | | | | |
| 24 mg IV | t_{1/2} | 12.28 | (3.3) | 12.3 | (9.04) |
| infusion | (h) | | | | |

¹data taken from Study B, a study of 24 mg given as a short infusion.
²data taken from Study A, a study of 12 mg given as SC.
Tₘₐₓ = Median (Min, Max) * Harmonic mean (harmonic SD)
*Harmonic mean (Jackknife SD), R = AUC₀₋₂₄ on Day 7/AUC₀₋₂₄ on Day 1, % Re₂₄ = % oral dose excreted in urine in 24 hr, NC = not computed

Tables 21 and 22 indicate that following oral and subcutaneous administrations, MNTX was readily absorbed with maximum MNTX plasma concentrations observed at 2 h and 0.25 h following oral dose and subcutaneous administration, respectively. Less than 4% of the orally administered dose was recovered in urine as an unconverted MNTX, markedly lower than the 31.5%-49.6% recovered in in urine following IV administration (Yuan et al. 2005 J Clin Pharm 45:538-546). Cross-study AUC_{inf} comparisons indicated that MNTX tablets demonstrated an absolute bioavailability of 4.24% (relative to IV infusion) and 3.7% bioavailability relative to SC injection whereas following multiple dose administration resulted in a slight increase in these values (higher AUC_{inf}) of 4.8% and 5.8% relative to SC and IV multiple dose administrations. Subcutaneous MNTX injection resulted in high bioavailability (112%) relative to short-term infusion.

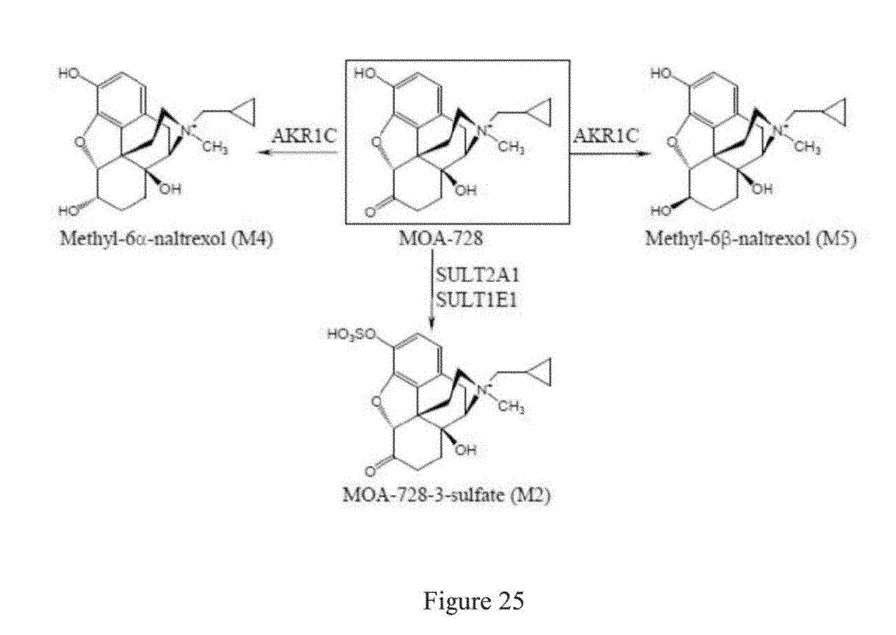

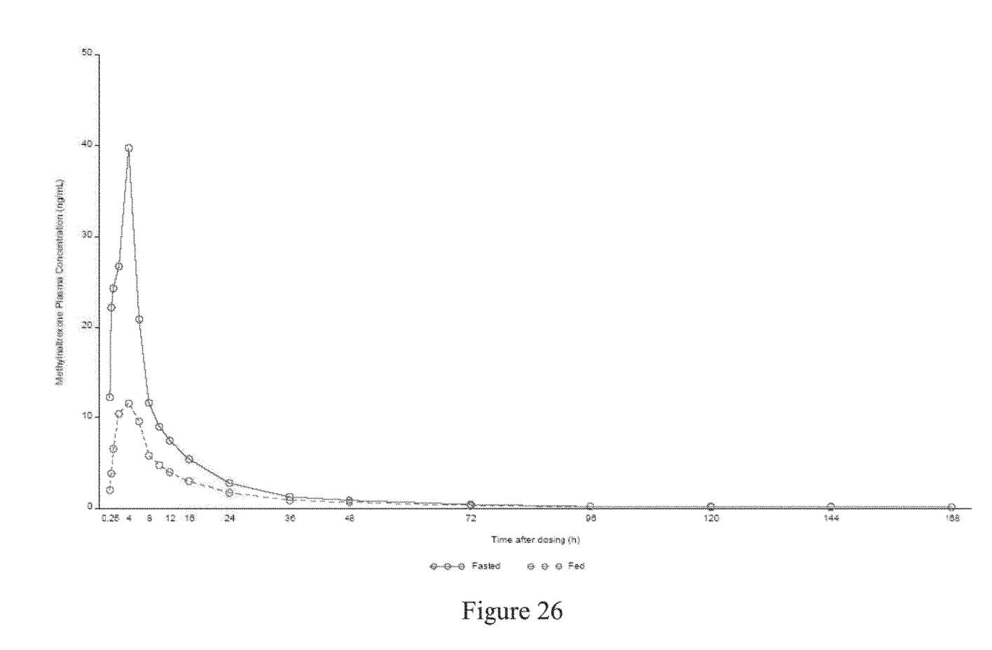

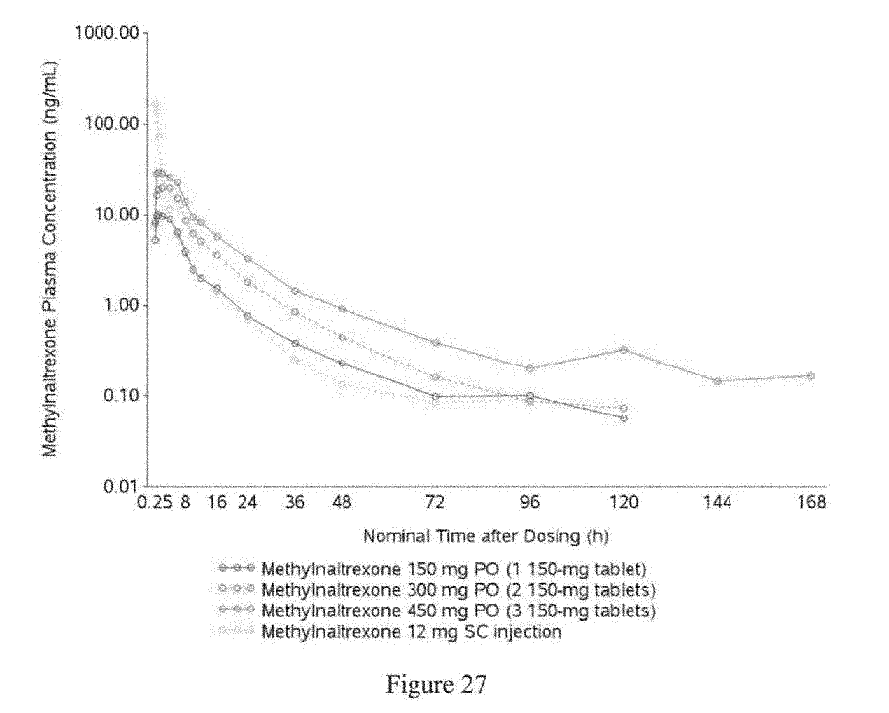

MNTX oral administration resulted in extensive metabolism, resulting in the formation methylnaltrexone sulfate (M2) and stereospecific hydroxylation to form 6α-(M4) and 6β-methylnaltrexol (M5) of which M4 was found to be the favored route of metabolite formation. Metabolic enzymes AKRC1C, SULT2A1 and SULT1E1 enzymes were reported be responsible for the MNTX metabolism into M2, M4 and M5 (FIG. 25).

No substantial differences in the average Cₘₐₓ and Tₘₐₓ were observed for MNTX and M2 between day 1 and day 7 for oral, SC or IV routes. These results indicate that the observed degree of accumulation (R) following multiple oral dose administration and reaching the apparent steady state was due to increased AUC values and decreased elimination which was evidenced by increased AUC_{inf} and delayed elimination t_{1/2} observed on Day 7 pharmacokinetics. Following subcutaneous administration, Cₘₐₓ and AUC_{inf} for MNTX and its metabolites were similar between Day 1 and Day 7, whereas following oral administration of MNTX tablets considerable increase in AUC and Cₘₐₓ were observed on Day 7 for MNTX and its metabolites. Higher accumulation for MNTX and its metabolite following multiple dose oral administration was evident from higher accumulation factor (R) values following oral dose (1.20 for MNTX, 1.30 for M2, 1.62 for M4 and 1.76 for M5) compared with the R values following subcutaneous administration (1.05 for MNTX, 1.13 for M2, 1.25 for M4 and 1.42 for M5). Following oral administration of MNTX, metabolite to MNTX ratios were higher for all three metabolites: 81.0% for M2, 54.21% for M4, and 29.78% for M5, compared to the lower metabolite-MNTX ratios following subcutaneous administration (29.30% for M2, 18.75% for M4, and 8.72% for M5).

In Study C, relative bioavailability of two methylnaltrexone formulations (film coated tablet and uncoated tablet) was evaluated using methylnaltrexone plasma pharmacokinetics and 90% CI approach. Mean plasma concentration-time profiles and results presented in Table 23 indicated that film coated methylnaltrexone tablets resulted in LSM (least squares mean) ratio between 90-105%. Intra-subject variability for MNTX formulations was between 29-36%.

TABLE 23
Relative Bioavailability of Two Methylnaltrexone (450 mg) tablets

| | Geometric Mean | | | | | 90% Confidence | | Intra- |
|---|---|---|---|---|---|---|---|---|
| | Film Coated | | Un-Coated | | LSM | Interval (CI) | | subject |
| | tablet | | Tablets | | ratio | Lower CI | Upper CI | CV |
| Cₘₐₓ (ng/mL) | 29.09 | | 31.32 | | 92.89 | 74.25 | 116.20 | 35.61 |
| AUCₜ | 278.86 | | 268.79 | | 103.75 | 86.22 | 124.83 | 29.14 |
| (ng*hr./mL) | | | | | | | | |
| AUC_{inf} | 285.47 | | 274.72 | | 103.91 | 86.51 | 124.82 | 28.86 |
| (ng*hr./mL) | | | | | | | | |
| Tₘₐₓ (hr.) | 2.00 | (0.5-6.00) | 1.00 | (0.5-6.0) | | | | |
| λ_{z} (1/hr.) | 0.0400 | (0.0166) | 0.0432 | (0.0117) | | | | |
| *T_{1/2} (hr.) | 17.33 | (7.40) | 16.04 | (4.30) | | | | |
| CL/F (L/hr.) | 1696.29 | (597.01) | 1706.88 | (549.30) | | | | |

Tₘₐₓ = Median (Min, Max)
*Harmonic mean (harmonic SD)

### Example 4: Clinical Pharmacokinetics of Orally Administered Methylnaltrexone with or without Food

The oral absorption of MNTX is limited. The estimated bioavailability of MNTX after oral administration was less than 1% in rats, and the relative oral bioavailability of MNTX enteric-coated tablets and enteric-coated granule-filled capsules was 2.27% and 2.43%, respectively, compared to the subcutaneous formulation in subjects on stable methadone maintenance.

The pharmacokinetics of MNTX tablets was highly variable among individuals, most likely a result of the low absorption and low systemic exposure after oral administration. The effect of food was investigated previously for MNTX formulated in immediate release (IR) tablet and IR capsule formulations. Following a high-fat meal, the mean Cₘₐₓ of MNTX decreased by 33% for the IR capsule formulation and approximately 45% for the IR tablet formulation; the AUC_{0-∞} decreased by 11% for the IR capsule formulation and by more than 30% for the IR tablet formulation. The median Tₘₐₓ and terminal t_{1/2} were not altered significantly by food.

The pharmacokinetics of MNTX in the oral 150 mg ion-pairing formulation has been investigated in 2 human studies.

A 2-part study was conducted in subjects on stable methadone maintenance therapy. In Part 1, patients received a single 150 mg dose of MNTX ion-pairing tablets; in Part 2, they received the same ion-pairing tablet dose in a crossover design compared with a single dose of MNTX IR tablets not using ion-pairing technology. Treatments with study drug were preceded by an overnight fast of ≥10 hours. For the ion-pairing tablets, the average Cₘₐₓ was 42.8 ng/mL with a median Tₘₐₓ of 1 hour and average AUC_{0-∞} was 180 hr·ng/mL in study part 1; the average Cₘₐₓ was 41.5 ng/mL with a median Tₘₐₓ of 2 hours and average AUC_{0-∞} was 176.8 hr·ng/mL in study part 2. The elimination t_{1/2} was variable with a mean value of 18.2 hours in part 1 and 25.5 hours in part 2.

A separate study evaluated the pharmacokinetics and pharmacodynamics of oral MNTX in subjects with chronic nonmalignant pain. The MNTX 150 mg tablets ion-pairing formulation was compared to MNTX 150 mg IR tablets formulation not using ion-pairing technology following fasting for 2 hours and 10 hours. Results for the MNTX 150 mg tablets ion-pairing formulation (10 hour fast) were the following: at 300 mg (2×150 mg tablets) and 450 mg (3×150 mg tablets), the average Cₘₐₓ was 32.5 and 54.7 ng/mL and AUC_{0-∞} was 156 hr·ng/mL and 223 hr·ng/mL, respectively.

Presented herein is a single-dose, 2-period crossover study to evaluate the effect of a standard high-fat breakfast on the pharmacokinetics of a single oral dose of 450 mg (3×150 mg tablets) MNTX. The study had 2 arms and 2 dosing periods.

Thirty-two subjects were enrolled into this study. Subjects were randomized at a 1:1 ratio to Arm 1 (fasted then fed) or Arm 2 (fed then fasted). Randomization was stratified by sex. Each subject received a single dose of MNTX 450 mg (administered as 3×150 mg tablets) with a high fat meal (MNTX fed) and after fasting (MNTX fasted). The fasted/fed study periods were separated by 7 days. The sequence of fasted/fed or fed/fasted dosing on Days 1 and 8 was determined by randomization on Day 1.

For the fasted treatment, a single 450 mg (3×150 mg) oral dose of MNTX tablets was administered to subjects following a supervised overnight fast of at least 10 hours. No food was allowed for at least 4 hours post dose.

For the fed treatment, a standard high-fat breakfast was given to the subjects following an overnight fast of at least 10 hours. A single 450 mg (3×150 mg) oral dose of MNTX tablets was administered to subjects 30 minutes after the subject began the meal. No food was allowed for at least 4 hours after drug administration.

Subjects remained at the clinical research unit (CRU) from Day 0 through Day 14 and were discharged on Day 15, which concluded their participation in the study.

Subjects were administered a single oral dose of MNTX tablets (450 mg) on Day 1 and Day 8 after a high fat meal or fasting as follows: (a) MNTX 450 mg (orally as 3×150 mg tablets) administered after a high-fat (high caloric) breakfast, or (b) MNTX 450 mg (orally as 3×150 mg tablets) administered after fasting.

Subjects received a single dose of MNTX 450 mg administered orally as 3×150 mg tablets immediately after a high fat/high calorie meal (MNTX fed) and after fasting (MNTX fasted). A 7-day washout period separated the fasted/fed crossover periods. The sequence of fasted/fed or fed/fasting dosing on Days 1 and 8 was determined by randomization on Day 1. Subjects fasted overnight for a minimum of 10 hours prior to administration of a high fat meal with the single dose of study drug (MNTX fed) or 10 hours prior to administration of the single dose of study drug (MNTX fasted).

Subjects were randomized to 1 of the 2 dosing sequences; the dosing sequences was based on a standard crossover design. The timing of the doses was determined by the length of the washout phase, which was calculated as 7 times the approximate t_{1/2} of oral MNTX observed in humans.

Each dose on Day 1 and 8 was administered with 240 mL of room temperature drinking water, and the subjects were instructed to drink all of the water. No food was permitted for 4 hours after drug administration and water was allowed as desired except for 1 hour before and after drug administration. Approximately 4 hours after dosing, a normal meal schedule could be resumed.

A high fat/high caloric meal includes fat content of approximately 50% of total calories in the meal (approximately 800 to 1000 calories total). Subjects receiving the MNTX fed treatment regimen were required to fast for at least 10 hours before breakfast and then to eat the protocol-specified breakfast starting 30 minutes before dosing. The high-fat, high caloric breakfast consisted of the following:
Two slices of toast with ½ pat of butter on each slice.
Two eggs fried in butter.
Two strips of bacon.
Four ounces (113 g) hash brown potatoes.
Eight ounces (240 mL) of whole milk.

The planned meal content was as follows:
Fat=500-600 calories, 50%.
Protein=150 calories.
Carbohydrate=250 calories.
Total calories=800 to 1000 calories.

The actual meal content received during the study is consistent with the FDA guidance on food effect studies, and included 972 total calories: 540 from fat, 299 from carbohydrates, and 125 from proteins. A normal meal schedule and diet was maintained, with the exceptions noted above.

Plasma concentrations of MNTX were determined using a validated analytical procedure involving high performance liquid chromatography with tandem quadrupole mass spectrometric detection. Blood samples for determination of MNTX concentrations in plasma were obtained predose (approximately 1 hour prior to dose administration) on Day 1, and at 0.25, 0.5, 1, 2, 4, 6, 8, 10, 12, 16, 24, 36, 48, 72, 96, 120, 144 and 168 hours following each dose administration on Days 1 and 8. Pharmacokinetic parameters that were measured and calculated include the following:

| Parameters | Description |
|---|---|
| Cₘₐₓ | Maximum observed plasma concentration |
| Tₘₐₓ | Time to maximum observed plasma concentration |
| | (time to Cₘₐₓ) |
| AUCₗₐₛₜ | Area under the plasma concentration versus time curve |
| | from time 0 (pre-dose) to the last quantifiable |
| | concentration-time point, calculated using the linear |
| | trapezoidal rule |
| AUC_{0-∞} | Area under the plasma concentration versus time curve |
| | from time 0 (pre-dose) to time infinity, calculated as |
| | the sum of AUCₗₐₛₜ and the last quantifiable plasma |
| | concentration/λ_{z} |
| λ_{z} | The terminal or disposition rate constant, calculated |
| | from the slope (by linear regression) of the terminal |
| | log-linear portion of the plasma versus time curve |
| t_{1/2} | Terminal or disposition half-life, calculated as ln2/λ_{z} |
| CL/F | Apparent oral clearance. |

Mean MNTX plasma concentration-time profiles following single oral 450 mg doses under fasted and fed conditions are shown in Table 24.

### MNTX Pharmacokinetic Parameters—Food Effect

Oral MNTX dosing in the fed state resulted in lower MNTX plasma concentrations when compared with dosing in the fasted state (Table 24 and Table 25). The arithmetic mean value for Cₘₐₓ in fed subjects was approximately one quarter (28%) of that measured for fasted subjects (12.91 ng/mL versus 45.55 ng/mL, respectively). Systemic exposure, as measured by AUCₗₐₛₜ and AUC_{0-∞}, was approximately 50% lower in fed subjects than in fasted subjects. Mean values for AUC_{0-∞} were 169.0 ng·h/mL in the fed state and 364.3 ng·h/mL in the fasted state. Median Tₘₐₓ was delayed in the fed state when compared with the fasted state (4.0 hr versus 2.0 hr, respectively). Oral clearance (CL/F) values were almost 2-fold higher under the fed state compared to the fasted state. The terminal rate constant was similar under fed and fasted conditions (λ_{z}=0.04 h⁻¹ for each), indicating that the terminal t_{1/2} of MNTX is similar when administered with or without food (approximately 17 h for each).

TABLE 24
Mean (±SD) Plasma Pharmacokinetic
Parameters of MNTX 450 mg: Food Effect

| | Single-Dose Fasted | | Single-Dose Fed | |
|---|---|---|---|---|
| Parameters | N = 32 | | N = 32 | |
| Cₘₐₓ (ng/mL) | 45.55 | (49.86) | 12.91 | (4.488) |
| Tₘₐₓ (h) ^{a} | 2.00 | (0.49-6.01) | 4.00 | (0.50-8.00) |
| AUCₗₐₛₜ (ng · h/mL) | 361.4 | (207.7) | 166.3 | (58.76) |
| AUC_{0-∞} (ng · h/mL) | 364.3 | (207.5) | 169.0 | (59.68) |
| CL/F (mL/h) | 1608644 | (788954.3) | 2961340 | (971027.8) |
| λ_{z} (h⁻¹) | 0.0403 | (0.0154) | 0.0413 | (0.0168) |
| t_{1/2} (h) ^{b} | 17.22 | (6.61) | 16.80 | (6.90) |

^{a} Median (range).
^{b} Harmonic mean (pseudo SD based on jackknife variance).

Table 25 presents results of statistical evaluations for bioequivalence for single-dose MNTX 450 mg, when administered under fasted (reference) and fed (test) conditions. For Cₘₐₓ, AUCₗₐₛₜ, and AUC_{0-∞}, the 90% CIs for the ratios of fasted to fed were outside of the accepted bioequivalence range of 80% to 125%, indicating nonbioequivalence under fed and fasted conditions. Systemic exposure parameters (Cₘₐₓ, AUCₗₐₛₜ, and AUC_{0-∞}) were higher in fasted subjects as compared with fed subjects.

TABLE 25
Cₘₐₓ and AUC Ratios and 90% CIs for MNTX 450
mg: Single-Dose Fasted versus Single-Dose Fed

| | Geometric | | | |
|---|---|---|---|---|
| | Least | Geometric | 90% CI for Geometric | |
| | Squares | Mean | Mean Ratios | |
| Parameters | Mean | Ratios^{a} | Lower^{a} | Upper^{a} |
| Cₘₐₓ (ng/mL) | | | | |
| Fasted | 33.37 | 273.62 | 222.59 | 336.34 |
| Fed | 12.20 | | | |
| AUCₗₐₛₜ (ng · h/mL) | | | | |
| Fasted | 313.9 | 199.28 | 173.27 | 229.20 |
| Fed | 157.5 | | | |
| AUC_{0-∞} (ng · h/mL) | | | | |
| Fasted | 317.2 | 198.17 | 172.45 | 227.71 |
| Fed | 160.0 | | | |

^{a}Ratio of fasted (reference) divided by fed (test), expressed as percentages.

Administration of a single, 450 mg dose of MNTX to healthy subjects under fed conditions resulted in a substantial decrease in systemic exposure when compared to MNTX administration under fasted conditions. Both AUCₗₐₛₜ and AUC_{0-∞} ratios were non-bioequivalent (90% CIs for fasted to fed ratios were outside the 80% to 125% range) and both parameters were approximately 2-fold higher in fasted as compared with fed subjects. Similarly, oral clearance values were almost 2-fold higher under the fed state compared to the fasted state.

In addition, the MNTX Cₘₐₓ was not bioequivalent between the fed and fasted states (e.g., geometric mean ratio=273.6%; 90% CI=222.6% to 336.3%). The arithmetic mean value for Cₘₐₓ in fed subjects was approximately one quarter (28%) of that measured for fasted subjects (12.91 ng/mL versus 45.55 ng/mL, respectively).

Median Tₘₐₓ was delayed in the fed state when compared with the fasted state (4.0 hr versus 2.0 hr, respectively).

The terminal rate constant was similar under fasted and fed conditions (λ_{z}=0.04 h⁻¹ for each), consistent with data indicating that the terminal t_{1/2} of MNTX is similar when administered with or without food (approximately 17 h for each).

Five of 32 subjects (16%) experienced at least 1 TEAE during the study. Four subjects had TEAEs during the fasted dosing period and 3 subjects had TEAEs during the fed dosing period. The most frequently experienced TEAE was headache (2 subjects, 6%). All TEAEs were considered mild by the investigator. No TEAEs were considered by the investigator to be related to MNTX. There were no deaths, SAEs, or TEAEs resulting in study discontinuation.

Minimal changes in laboratory test results were observed for subjects during the course of the study. No laboratory test result was considered by the investigator to be a TEAE.

No significant effect of MNTX on cardiac safety parameters or vital signs was observed in this trial.

Results of other studies show that the pharmacokinetics of orally administered MNTX are characterized by low bioavailability, limited tissue distribution outside the GI tract (including restricted central nervous system exposure), and low plasma protein binding. Peak plasma concentration and AUC appear to increase with increasing dose.

The effects of a high-fat meal on the pharmacokinetics of a single 450-mg oral dose of MNTX observed in this study are consistent with those previously observed for other oral formulations of MNTX (IR tablet and capsule). In a prior study, fasting increased systemic absorption of MNTX by approximately 25%. In the current study, the presence of food significantly delayed MNTX absorption (e.g., increased Tₘₐₓ), and decreased MNTX systemic exposure by approximately half to three quarters (as determined by AUC and Cₘₐₓ). Oral MNTX was not bioequivalent between fasted and fed states.

Laxation effects of MNTX were also increased in fasted subjects compared to non-fasted subjects in a phase 3 study \ following the first dose of study drug. This result suggests that the therapeutic efficacy of MNTX is correlated with the extent of systemic absorption.

Although fasting increased systemic exposure to MNTX, the incidences of TEAEs were similar between fed and fasted conditions. A single dose of MNTX 450 mg was well tolerated; TEAEs were reported by 5 subjects, and all were mild in intensity.

### Example 5: Clinical Pharmacokinetics of Oral Administration of Methylnaltrexone Compared to Subcutaneous Administration of the Same

The oral dosage levels and formulation of MNTX evaluated here were the same as those in a phase 3 study of oral MNTX tablets, with the exception of a nonfunctional coating on the MNTX tablets. This nonfunctional coating is comprised of inactive ingredients polyvinyl alcohol, polyethylene glycol, and titanium dioxide. The pharmacokinetics of the uncoated tablet used in the phase 3 study and the coated tablets used in the current study were compared in a separate study. The current study was designed to evaluate the comparative bioavailability of orally administered, 150, 300, and 450 mg MNTX doses versus a 12 mg subcutaneous (SC) injection of MNTX. A single-dose pharmacokinetic profile of oral MNTX tablets was also planned for evaluation in this study.

The objectives of this study were to evaluate the comparative bioavailability of 150, 300, and 450 mg single oral doses of MNTX tablets versus a 12 mg single SC dose of MNTX, and to characterize the pharmacokinetics of MNTX tablets after single oral dose administration in healthy subjects.

Presented herein is a randomized, open-label, crossover study consisting of 6 dosing sequences, each with 2 dosing periods; the dosing periods were separated by 7 days. All subjects were housed in the clinical research unit from Day −1 through Day 14 and were discharged on Day 15, which concluded their participation in the study. Prior to receiving study drug on Days 1 and 8, the subjects underwent an overnight fast of at least 10 hours, beginning on Days 0 and 7, respectively. In both dosing periods, the subjects received a single oral dose of MNTX tablets (150, 300, or 450 mg) or a single SC injection of MNTX (12 mg). The dosing was conducted in a crossover fashion (e.g., a tablet was administered at one visit and a SC injection was administered at the alternate visit). The strength of oral methylnaltrexone dose (150 mg, 300 mg, or 450 mg) and the dosing sequence (Day 1: oral tablet; Day 8: SC injection vs the alternate dosing order) for each subject were determined by random assignment. Each oral dose was administered with 240 mL of room temperature drinking water. The subjects were instructed to drink all of the water and were told to swallow the tablets whole (e.g., not to chew, divide, or crush them). Blood samples were collected for pharmacokinetic analyses prior to dosing (approximately 1 hour prior) on Day 1, and at 0.25, 0.5, 1, 2, 4, 6, 8, 10, 12, 16, 24, 36, 48, 72, 96, 120, 144, and 168 hours after dosing on Days 1 and 8.

Each tablet contained 150 mg of the active pharmaceutical ingredient, MNTX. In addition, each tablet contained the following inactive ingredients: colloidal silicon dioxide, crospovidone, edetate disodium calcium dihydrate, magnesium stearate, microcrystalline cellulose, polysorbate 80, siliconized microcrystalline cellulose, sodium bicarbonate, sodium lauryl sulfate, and talc.

Each injection vial contained 12 mg of the active pharmaceutical ingredient, MNTX, per 0.6 mL of solution (i.e., 20 mg/mL solution). The formulation also contained the following inactive ingredients: edetate calcium disodium, sodium chloride, glycine hydrochloride, and sodium hydroxide.

In this study, all 48 enrolled subjects received study drug in each of the 2 study periods and were included in the safety and pharmacokinetic analyses.

The mean Cₘₐₓ for MNTX was observed at 15 minutes following 12 mg SC injection and plasma concentrations then diminished rapidly within the initial postdosing period (Table 26; abbreviations: PO=per oral, SC=subcutaneous). Beginning around 4 hours postdosing and continuing through at least 72 hours postdosing, there were greater mean plasma concentrations of MNTX following oral MNTX dosing relative to the SC injection for the 300 mg and 450 mg doses, but not for the 150 mg oral dose.

Single-dose pharmacokinetic parameters of SC MNTX compared with oral MNTX demonstrated that Cₘₐₓ was 4- to 13-fold higher, Tₘₐₓ was 6- to 8-fold shorter, and t_{1/2} was shorter by 5 to 7 hours following SC MNTX 12 mg versus oral MNTX 150, 300, and 450 mg (Table 26).

Systemic exposure to MNTX as measured by Cₘₐₓ and AUC followed generally linear, dose-dependent trends among the oral doses (Table 26). Mean AUC and Cₘₐₓ values increased with increasing single oral doses of MNTX tablets from 150 mg to 450 mg; Cₘₐₓ increased from 13.2 to 39.9 ng/mL and AUC_{0-∞} increased from 106.9 to 373.3 ng·h/mL at MNTX 150 mg and MNTX 450 mg, respectively. Median Tₘₐₓ values were constant, ranging from approximately 1.5 to 2.0 hours post dosing. The mean CL/F values were also similar across oral dosing groups. The mean t_{1/2} increased slightly from 14.0 hours to 16.6 hours as the oral MNTX doses increased, respectively, from 150 mg to 450 mg.

The Cₘₐₓ occurred more rapidly following administration of the SC injection (median Tₘₐₓ=15 minutes) than following any of the oral study drug administrations (median Tₘₐₓ ranged from 1.5 to 2.0 hours) (Table 26).

Comparison of systemic exposure parameters (Cₘₐₓ and AUC) demonstrates at least 4-fold higher mean Cₘₐₓ following SC MNTX 12 mg versus each of the oral MNTX doses; however, mean AUC_{0-∞} following SC MNTX 12 mg was only 16% higher versus oral MNTX 300 mg and 28% lower versus oral MNTX 450 mg (Table 26). Mean Cₘₐₓ values were 174.0 ng/mL following SC MNTX 12 mg versus 26.2 and 39.9 ng/mL following oral MNTX 300 mg and 450 mg, respectively; and mean AUC_{0-∞} values following SC MNTX 12 mg were 269.1 versus 231.2 and 373.3 ng·h/mL following oral MNTX 300 mg and 450 mg, respectively.

Further, consistent with the observed differences in Cₘₐₓ and AUC between SC MNTX 12 mg and oral MNTX 450 mg, 300 mg, or 150 mg, elimination of MNTX was faster following SC versus oral administration (Table 26). The MNTX clearance rate (CL/F) was faster, 45698.7 versus 1664001.3 mL/h, and the t_{1/2} value was shorter, 9.2 versus 16.6 hours, for SC MNTX 12 mg compared with oral MNTX 450 mg.

TABLE 26
Single-Dose Pharmacokinetic Parameters for Oral MNTX (150, 300, and 450 mg) and Subcutaneous MNTX (12 mg)

| | MNTX 150 mg | | MNTX 300 mg | | MNTX 450 mg | | MNTX 12 mg | |
|---|---|---|---|---|---|---|---|---|
| | Tablet | | Tablet | | Tablet | | SC Injection | |
| | (N = 16) | | (N = 16) | | (N = 16) | | (N = 48) | |
| Cₘₐₓ (ng/mL) | 13.22 | (15.17) | 26.22 | (18.40) | 39.89 | (32.11) | 174.01 | (61.42) |
| Mean (standard deviation) | | | | | | | | |
| AUC_{0-∞} (ng · h/mL) | 106.87 | (64.77) | 231.24 | (115.98) | 373.32 | (207.36) | 269.09 | (45.14) |
| Mean (standard deviation) | | | | | | | | |
| AUC₀₋ₜ (ng · h/mL) | 104.65 | (64.66) | 229.37 | (116.27) | 366.68 | (205.71) | 267.87 | (44.94) |
| Mean (standard deviation) | | | | | | | | |
| Tₘₐₓ (h) | 2.00 | (0.45, 6.00) | 1.50 | (0.50, 6.00) | 2.00 | (0.50, 6.00) | 0.25 | (0.25, 0.68) |
| Median (minimum, maximum) | | | | | | | | |
| CL/F (mL/h) | 1735472.22 | (683440.65) | 1564638.99 | (627269.81) | 1664001.28 | (1035943.18) | 45698.71 | (6902.56) |
| Mean (standard deviation) | | | | | | | | |
| t_{1/2} (h) ^{a} | 13.95 | (5.51) | 14.16 | (4.71) | 16.57 | (4.42) | 9.16 | (2.03) |
| Mean (standard deviation) | | | | | | | | |

Abbreviations: AUC_{0-∞} = area under the plasma concentration versus time curve from time 0 (predose) to time infinity; AUC₀₋ₜ = AUC from time 0 (predose) to the last quantifiable concentration-time point; Cₘₐₓ = maximum observed plasma concentration; CL/F = apparent oral clearance; MNTX = methylnaltrexone; SC = subcutaneous; Tₘₐₓ = time to Cₘₐₓ; t_{1/2} = terminal or disposition half-life.
Note:
Mean values are arithmetic means unless otherwise specified.
^{a} Expressed as harmonic means and pseudo standard deviation based on jackknife variance.

Oral MNTX 450 mg resulted in a Cₘₐₓ that was approximately 20% of the Cₘₐₓ from SC MNTX 12 mg and an AUC_{0-∞} that was approximately 123% of the AUC_{0-∞} from SC MNTX 12 mg; the geometric mean ratios of the oral tablet (test) to the SC injection (reference) were 20.0% for Cₘₐₓ and 123.2% for AUC_{0-∞} (Table 27). The lower bound of the 90% confidence interval for Cₘₐₓ (4.3%) was well below 80% and the upper bound of the 90% confidence interval for AUC_{0-∞} (150.7%) was greater than 125% indicating that both parameters were nonbioequivalent by the 80% to 125% rule.

Also, the Cₘₐₓ values were approximately 13% and 6% following oral MNTX 300 mg and 150 mg, respectively, of the Cₘₐₓ following SC MNTX 12 mg, and the AUC_{0-∞} values following these oral doses were approximately 75% and 36%, respectively, of the AUC_{0-∞} following SC MNTX 12 mg (geometric mean ratios in Table 27). The 90% confidence intervals of the Cₘₐₓ and AUC_{0-∞} geometric mean ratios indicated nonbioequivalence of the 300 mg and 150 mg oral doses with SC MNTX 12 mg by the 80% to 125% rule (lower bounds of the 90% confidence intervals were <80%).

The bioavailability of oral MNTX relative to SC MNTX, comparing arithmetic mean AUC_{0-∞} values for oral MNTX 450 mg to SC MNTX 12 mg, was 3.7% (normalized to dose in mg/kg [assuming mean of 81 kg body weight, based on subject mean demographics] by the following calculation: 373.3 ng·h/mL/[450 mg/81 kg]÷269.1 ng·h/mL/[12 mg/81 kg]×100). Dose-normalized bioavailability of oral MNTX relative to SC MNTX for the 300 mg and 150 mg doses were 3.4% and 3.2%, respectively.

TABLE 27
Geometric Mean Ratios and 90% Confidence Intervals for Oral MNTX to
SC MNTX Systemic Exposure Parameters (Pharmacokinetic Population)

| | | Geometric | | 90% CI for GMR | |
|---|---|---|---|---|---|
| Parameter | Treatment | LSM | GMR (%) | Lower (%) | Upper (%) |
| Cₘₐₓ | MNTX 150 mg Tablet | 9.466405 | 5.7788363 | 4.3427666 | 7.6897866 |
| (ng/mL) | MNTX 300 mg Tablet | 21.767989 | 13.288428 | 9.9861877 | 17.682657 |
| | MNTX 450 mg Tablet | 32.698217 | 19.960866 | 15.000491 | 26.561542 |
| | MNTX 12 mg SC Injection | 163.81161 | | | |
| AUC₀₋ₜ | MNTX 150 mg Tablet | 94.197517 | 35.614388 | 29.035654 | 43.683694 |
| (ng · h/mL) | MNTX 300 mg Tablet | 197.65641 | 74.730334 | 60.926054 | 91.662309 |
| | MNTX 450 mg Tablet | 321.18884 | 121.43573 | 99.003968 | 148.94994 |
| | MNTX 12 mg SC Injection | 264.49287 | | | |
| AUC_{0-∞} | MNTX 150 mg Tablet | 96.732071 | 36.405386 | 29.758719 | 44.5366 |
| (ng · h/mL) | MNTX 300 mg Tablet | 199.76822 | 75.18333 | 61.456828 | 91.975672 |
| | MNTX 450 mg Tablet | 327.35332 | 123.20034 | 100.70719 | 150.71738 |
| | MNTX 12 mg SC Injection | 265.70813 | | | |

Abbreviations: CI = confidence interval; GMR = geometric means ratio calculated as the tablet/injection × 100; LSM = least squares mean; MNTX = methylnaltrexone bromide; SC = subcutaneous.

Systemic exposure to MNTX as measured by Cₘₐₓ and AUC followed generally linear, dose-dependent trends among the oral doses. Mean AUC and Cₘₐₓ values increased with increasing single oral doses of MNTX tablets from 150 mg to 450 mg; Cₘₐₓ increased from 13.2 to 39.9 ng/mL and AUC_{0-∞} increased from 106.9 to 373.3 ng·h/mL at MNTX 150 mg and MNTX 450 mg doses, respectively.

The Cₘₐₓ occurred more rapidly following administration of the 12 mg SC MNTX injection (median Tₘₐₓ=15 minutes) than following any of the oral study drug administrations (median Tₘₐₓ ranged from 1.5 to 2.0 hours).

Comparison of systemic exposure parameters (Cₘₐₓ and AUC) demonstrates 4- to 13-fold higher mean Cₘₐₓ following SC MNTX 12 mg versus each of the oral MNTX doses; however, mean AUC_{0-∞} following SC MNTX 12 mg was only 16% higher versus oral MNTX 300 mg and 28% lower versus oral MNTX 450 mg. Mean Cₘₐₓ values were 174.0 ng/mL following SC MNTX 12 mg versus 26.2 and 39.9 ng/mL following oral MNTX 300 mg and 450 mg, respectively; and mean AUC_{0-∞} values were 269.1 following SC MNTX 12 mg versus 231.2 and 373.3 ng·h/mL following oral MNTX 300 mg and 450 mg, respectively.

Calculation of the geometric mean ratios for oral MNTX tablets (test) relative to the SC MNTX injection (reference) indicated that the Cₘₐₓ from an oral MNTX 450 mg dose was approximately 20% of that observed for the 12 mg SC MNTX injection and the AUC_{0-∞} from an oral MNTX 450 mg dose was approximately 123% of that observed from the 12 mg SC MNTX injection. Also, the Cₘₐₓ values were approximately 13% and 6% following oral MNTX 300 mg and 150 mg, respectively, of the Cₘₐₓ following SC MNTX 12 mg, and the AUC_{0-∞} values were approximately 75% and 36% following these oral doses, respectively, of the AUC_{0-∞} following SC MNTX 12 mg.

Consistent with the observed differences in Cₘₐₓ and AUC between the 12 mg SC MNTX injection and the oral MNTX 450 mg, 300 mg, and 150 mg doses, elimination of MNTX was faster following SC injection versus oral administration: the MNTX clearance rate (CL/F) was faster, 45698.7 versus 1664001.3 mL/h, and the t_{1/2} value was shorter, 9.2 versus 16.6 hours, for the 12 mg SC MNTX injection compared with the oral MNTX 450 mg dose.

The dose-normalized bioavailability of oral MNTX relative to SC MNTX injection, comparing arithmetic mean AUC_{0-∞} values for an oral MNTX 450 mg, 300 mg, or 150 mg dose to the 12 mg SC MNTX injection, were 3.7%, 3.4%, and 3.2%, respectively.

This was a phase 1, randomized, open-label, crossover study consisting of 6 dosing sequences, each with 2 dosing periods. In both dosing periods, the subjects received a single oral dose of MNTX tablets (150, 300, or 450 mg) or a single SC injection of MNTX (12 mg). The dosing was conducted in a crossover fashion (i.e., a tablet was administered at one visit and a SC injection was administered at the alternate visit).

Forty-eight subjects were enrolled and 47 subjects (97.9%) completed the study; one subject discontinued due to personal reasons after receiving study drug in both study periods. The subjects received study drug in accordance with the randomization schedule; specifically, 16 subjects each received a single oral dose of 150, 300, and 450 mg MNTX tablets and all 48 subjects received a single 12 mg SC injection of MNTX.

Single-dose pharmacokinetic parameters of SC MNTX compared with oral MNTX demonstrated that Cₘₐₓ was 4- to 13-fold higher, Tₘₐₓ was 6- to 8-fold shorter, and t_{1/2} was shorter by 5 to 7 hours following SC MNTX 12 mg versus oral MNTX 150, 300, and 450 mg.

Systemic exposure to MNTX as measured by Cₘₐₓ and AUC (both AUCₗₐₛₜ and AUC_{0-∞}) followed generally linear, dose-dependent trends among the oral doses.

Comparison of systemic exposure parameters (Cₘₐₓ and AUC) demonstrates at least 4-fold higher Cₘₐₓ following SC MNTX 12 mg versus each of the oral MNTX doses; however, mean AUC_{0-∞} following SC MNTX 12 mg was only 16% higher versus oral MNTX 300 mg and 28% lower versus oral MNTX 450 mg. The Tₘₐₓ was shorter following SC MNTX 12 mg (15 minutes) than following oral MNTX 150 mg 300 mg, or 450 mg, (2, 1.5, and 2 hours, respectively). Also, consistent with the observed differences in Cₘₐₓ and AUC, the t_{1/2} value was shorter, 9.2 versus 16.6 hours, for SC MNTX 12 mg compared with oral MNTX 450 mg (t_{1/2} were 14.2 and 14.0 hours following oral MNTX 300 mg and 150 mg, respectively).

The single-dose pharmacokinetics of oral MNTX 150 mg tablet (ion-pairing) formulation was also studied in a recent study of healthy adults and in prior studies of subjects with noncancer pain and OIC and subjects on stable methadone maintenance. The single-dose pharmacokinetic parameters of oral MNTX were generally similar in the current study and in these other studies, although there were some quantitative differences in Cₘₐₓ and AUC in the current study and recent study of healthy adults when compared with prior studies of subjects with noncancer pain and OIC and of subjects on stable methadone maintenance.

Methylnaltrexone by SC injection was compared to MNTX administered orally in a pharmacokinetic study in subjects on stable methadone maintenance. The oral MNTX formulation was different in the current study than in the previous study, in which the oral formulations were enteric-coated granules in capsules and enteric-coated tablets. Although it is difficult to compare the current study and the previous study due to different oral MNTX formulations, the comparative pharmacokinetic profiles between SC dosing and oral dosing were similar between studies. Specifically, as in the current study, Tₘₐₓ was shorter, Cₘₐₓ was higher, and t_{1/2} was shorter following SC dosing compared with oral dosing; whereas differences in AUC values between SC and oral administrations were less pronounced than the differences in Cₘₐₓ, Tₘₐₓ, and t_{1/2}. Dose-normalized oral bioavailability relative to SC injection was 2.43% for enteric-coated capsules and 2.27% for enteric-coated tablets in the previous study, compared with 3.7% for the oral tablet (ion-pairing) formulation in the current study.

Oral doses of 150, 300, and 450 mg MNTX tablets and 12 mg MNTX SC injection and well tolerated in healthy volunteers who received 1 of the 3 oral doses of MNTX tablets as well as the SC injection of MNTX in this 2-period crossover study.

One skilled in the art will readily ascertain the essential characteristics of the invention and understand that the foregoing description and Examples are illustrative of practicing the provided invention. Those skilled in the art will be able to ascertain using no more than routine experimentation, many variations of the detail presented herein may be made to the specific embodiments of the invention described herein without departing from the spirit and scope of the present invention.

1. A method of treating a subject having opioid induced constipation, comprising orally administering to the subject a pharmaceutical composition comprising a salt of formula (I): 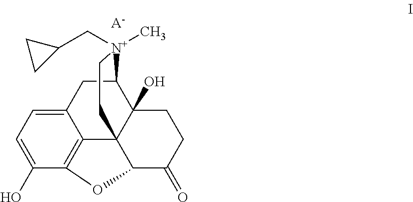
wherein A⁻ is an anion of an amphiphilic pharmaceutically acceptable excipient, wherein the administration of the pharmaceutical composition results in a rescue free bowel movement; thereby treating the subject.

2. (canceled)

3. The method of claim 1, wherein A⁻ is sodium dodecyl (lauryl) sulfate.

4. The method of claim 1, wherein the pharmaceutical composition comprises a combination of a first salt comprising methylnaltrexone and bromide, and a second salt comprising methylnaltrexone and sodium dodecyl (lauryl) sulfate.

5. (canceled)

6. The method of claim 1, wherein the pharmaceutical composition further comprises at least one agent selected from the group consisting of sodium bicarbonate, microcrystalline cellulose, crospovidone, polysorbate 80, edetate calcium disodium dehydrate, silicified microcrystalline cellulose, talc, colloidal silicon dioxide, magnesium stearate, and combinations thereof.

7. The method of claim 1, wherein the pharmaceutical composition is a tablet.

8. The method of claim 1, comprising orally administering about 150 mg, 300 mg or 450 mg of methylnaltrexone, or a salt thereof.

9. The method of claim 8, wherein the about 150 mg of methylnaltrexone, or a salt thereof, is administered as one tablet comprising about 150 mg of methylnaltrexone bromide, the about 300 mg of methylnaltrexone, or a salt thereof, is administered as two tablets each comprising about 150 mg of methylnaltrexone bromide, or the about 450 mg of methylnaltrexone, or a salt thereof, is administered as three tablets each comprising about 150 mg of methylnaltrexone bromide.

10-13. (canceled)

14. The method of claim 1, wherein the subject has chronic non-malignant pain, optionally for at least 2 months prior to administration of the pharmaceutical composition.

15. (canceled)

16. The method of claim 1, wherein the subject has been receiving opioid treatment prior to administration of the pharmaceutical composition, optionally for at least one month.

17. (canceled)

18. The method of claim 1, wherein the subject has been receiving opioid treatment comprising at least 50 mg of oral morphine equivalents per day for at least 14 days.

19. (canceled)

20. The method of claim 1, wherein the subject has had opioid induced constipation for at least 30 days.

21. The method of claim 1, wherein the subject has experienced less than 3 rescue free bowel movements per week for at least four consecutive weeks, straining during bowel movements, incomplete evacuation, or a Bristol Stool Form Scale type 1 or 2 for at least 25% of rescue free bowel movements.

22-24. (canceled)

25. The method of claim 1, wherein the method results in (i) a rescue free bowel movement within 4 hours of administration of the pharmaceutical composition; (ii) an increase of at least one, two, three, four or five rescue free bowel movements per week as compared to the number of rescue free bowel movements per week prior to administration of the pharmaceutical composition; or (iii) an increase of at least one rescue free bowel movement per week for each of the first 4 weeks of daily administration of the pharmaceutical composition.

26-28. (canceled)

29. The method of claim 1, wherein (i) the subject experiences at least 3 rescue free bowel movements in each of the first 4 weeks of daily administration of the pharmaceutical composition; and (ii) the subject experiences an increase of at least one rescue free bowel movement per week for at least 3 of the first 4 weeks of daily administration as compared to the number of rescue free bowel movements per week prior to administration of the pharmaceutical composition.

30-36. (canceled)

37. The method of claim 1, further comprising identifying if the subject:
(i) has chronic non-malignant pain;
(ii) has had chronic non-malignant pain for at least 2 months;
(iii) has been receiving opioid treatment;
(iv) has been receiving opioid treatment for at least one month;
(v) has been receiving opioid treatment comprising at least 50 mg of oral morphine equivalents per day for at least 14 days;
(vi) has opioid induced constipation;
(vii) has had opioid induced constipation for at least 30 days;
(viii) has had less than 3 rescue free bowel movements per week for at least four consecutive weeks;
(ix) has experienced straining during bowel movements;
(x) has experienced incomplete evacuation;
(xi) has experienced a Bristol Stool Form Scale type 1 or 2 for at least 25% of rescue free bowel movements;
(xii) has no history of chronic constipation prior to initiation of opioid therapy; or
(xiii) any combination of (i)-(xii); and
orally administering to the subject a pharmaceutical composition comprising a salt of formula (I): 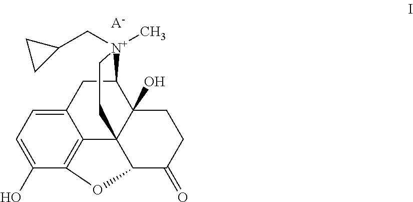
wherein A⁻ is an anion of an amphiphilic pharmaceutically acceptable excipient, wherein the subject exhibits any one of (i)-(x).

38-49. (canceled)

50. The method of claim 1, comprising the steps of
(a) orally administering to the subject the pharmaceutical composition comprising about 150 mg of methylnaltrexone, or a salt thereof, and sodium dodecyl (lauryl) sulfate;
(b) determining whether the composition treats the subject, wherein at least one response selected from the group consisting of (i)-(iii) indicates that the composition treats the subject:
(i) a rescue free bowel movement within four hours of administration of the pharmaceutical composition;
(ii) an increase in the number of rescue free bowel movements per week upon daily administration of the pharmaceutical composition as compared to the number of rescue free bowel movements per week prior to daily administration of the pharmaceutical composition; or
(iii) an increase in the number of rescue free bowel movements per week upon daily administration of the pharmaceutical composition as compared to the number of rescue free bowel movements per week prior to administration of the pharmaceutical composition in at least three of the first four weeks of daily administration; and at least three rescue free bowel movements per week for the first four weeks of daily administration;
(c) orally administering a pharmaceutical composition comprising 300 mg or 450 mg of methylnaltrexone, or a salt thereof, and sodium dodecyl (lauryl) sulfate, if the subject does not exhibit a response selected from the group consisting of (b)(i)-(iii) following step (a).

51. The method of claim 1,
wherein the composition provides a dose in the range of about 300 mg to about 400 mg of methylnaltrexone or salt thereof; wherein (i) the method results in a rescue free bowel movement within 4 hours of administration of the pharmaceutical composition; and (ii) the result is sustainable for at least 4 weeks with daily administration.

52. The method according to claim 51, wherein the method further provides the subject (i) at least 3 rescue free bowel movements per week for at least 3 of 4 weeks of daily administration of the pharmaceutical composition; and (ii) the subject experiences an increase of at least one rescue free bowel movement per week as compared to the number of rescue free bowel movements per week prior to administration of the pharmaceutical composition.

53-62. (canceled)

63. A method of increasing the bioavailability of MNTX, comprising administering MNTX without food to a subject in need thereof.

64-74. (canceled)

75. A method of increasing the laxation effect of MNTX, comprising administering MNTX without food to a subject in need thereof.

76-81. (canceled)
